SEQUENCE LISTING (19 sequences)

SEQ ID NO: 1 - DNA, 20 bp, synthetic construct
  source 1..20: mol_type = other DNA
aaggagtcacacgtgtgcta

SEQ ID NO: 2 - DNA, 20 bp, synthetic construct
  source 1..20: mol_type = other DNA
acctcaagatcaaggaaagg

SEQ ID NO: 3 - DNA, 20 bp, synthetic construct
  source 1..20: mol_type = other DNA
acgcagcctgcccttggaca

SEQ ID NO: 4 - DNA, 20 bp, synthetic construct
  source 1..20: mol_type = other DNA
caagatcaaggaaaggagga

SEQ ID NO: 5 - DNA, 20 bp, synthetic construct
  source 1..20: mol_type = other DNA
caggcctggggttgggcatc

SEQ ID NO: 6 - DNA, 20 bp, synthetic construct
  source 1..20: mol_type = other DNA
ccaggcctggggttgggcat

SEQ ID NO: 7 - DNA, 20 bp, synthetic construct
  source 1..20: mol_type = other DNA
ctgacctcaagatcaaggaa

SEQ ID NO: 8 - DNA, 20 bp, synthetic construct
  source 1..20: mol_type = other DNA
ctgcgtagacaataggggaa

SEQ ID NO: 9 - DNA, 20 bp, synthetic construct
  source 1..20: mol_type = other DNA
gagtcacacgtgtgctaggg

SEQ ID NO: 10 - DNA, 20 bp, synthetic construct
  source 1..20: mol_type = other DNA
ggcaggctgcgtagacaata

SEQ ID NO: 11 - DNA, 20 bp, synthetic construct
  source 1..20: mol_type = other DNA
ggcatcgggtccttgtccaa

SEQ ID NO: 12 - DNA, 20 bp, synthetic construct
  source 1..20: mol_type = other DNA
tcgggtccttgtccaagggc

SEQ ID NO: 13 - DNA, 20 bp, synthetic construct
  source 1..20: mol_type = other DNA
ttggacaaggacccgatgcc

SEQ ID NO: 14 - DNA, 20 bp, synthetic construct
  source 1..20: mol_type = other DNA
ttgtctacgcagcctgccct

SEQ ID NO: 15 - DNA, 128 bp, synthetic construct
  source 1..128: mol_type = other DNA
agatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 16 - DNA, 128 bp, synthetic construct
  source 1..128: mol_type = other DNA
aacttgtttattgcagcttataatggttacaaataaagcaatagcatcacaaatttcacaaataaagcatttttttcactgcattctagttgtggtttgtccaaactcatcaatgtatcttatcatct

SEQ ID NO: 17 - DNA, 379 bp, synthetic construct
  source 1..379: mol_type = other DNA
gataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttagatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 18 - protein, 5 aa, synthetic construct
GGGGS

SEQ ID NO: 19 - DNA, 105 bp, synthetic construct
  source 1..105: mol_type = other DNA
ctcaggtggtcgagtatctcataccgccctagcacacgtgtgactcctttcccctattgtctacgcagcctgcccttggacaaggacccgatgcccaaccccagg

(19) United States
(12) Patent Application Publication
(10) Pub. No.: US 2026/0265320 A1
(43) Pub. Date: Sep. 10, 2026

(54) COMPOSITIONS AND METHODS FOR ENGINEERING CELLS

(71) Applicant: Sonoma Biotherapeutics, Inc., South San Francisco, CA (US)

(72) Inventors: Olivier HUMBERT, Seattle, WA (US); Lee SHAFFER, Seattle, WA (US); Joshua BEILKE, Issaquah, WA (US)

(21) Appl. No.: 19/497,124

(22) PCT Filed: Jun. 24, 2024
(86) PCT No.: PCT/US2024/035322

§ 371 Date: Dec. 24, 2025

Related U.S. Application Data
(60) Provisional application No. 63/523,070, filed on Jun. 24, 2023.

(51) Int. Cl. C07K 14/47; A61K 40/11; A61K 40/31; A61K 40/41; C07K 16/18; C07K 16/28; C12N 9/22; C12N 15/11; C12N 15/90

(52) U.S. Cl. CPC C07K 14/4702; A61K 40/11; A61K 40/31; A61K 40/416; C07K 16/18; C07K 16/2803; C12N 9/222; C12N 15/11; C12N 15/907; C12N 2310/20

(57) ABSTRACT

Autoimmune disease are conditions arising from an abnormal, inflammatory immune response in a normally functioning part of the body. Provided herein are compositions and methods for genetically engineering cells, improvements in the generation of therapeutic cells, improvements in selective expression of one or more payloads in a target cells. Examples include genetic engineering of immune cells, preferably immunosuppressive cells, e.g., Tregs, among others to shift the balance from an inflammatory to a more tolerogenic microenvironment in areas of active inflammation when delivered to a patient suffering from an autoimmune disease.

### CROSS-REFERENCE

This application claims the benefit of U.S. Provisional Application No. 63/523,070, filed Jun. 24, 2023, which application is incorporated herein by reference.

### STATEMENT AS TO FEDERALLY FUNDED RESEARCH

None.

### BACKGROUND

Autoimmune diseases, are conditions arising from an abnormal immune response to a functioning body part. There has been much interest in the use of immunosuppressive cells, e.g., regulatory T cells (Tregs), to treat autoimmune diseases. A number of approaches have been explored to boost Treg numbers and functions in order to treat autoimmune diseases. However, none of these approaches have been as effective as hoped. Therefore, there is growing need for development of new compositions and methods for engineering Treg cells.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a schematic of Treg functionality.

FIG. 2 shows a method for inserting, for example using CRISPR/Cas, a polynucleotide in the FOXP3 locus and the resulting polynucleotide in the FOXP3 locus.

FIG. 3 shows a method for inserting, for example using CRISPR/Cas, a polynucleotide in the FOXP3 locus and the resulting polynucleotide in the FOXP3 locus.

FIG. 4 shows an illustration of insertion of a portion of a donor template (403) comprising a CAR and a 2A peptide into the FOXP3 locus (401) under the control of a FOXP3 promoter (403). The resulting polynucleotide (404) comprising a start codon, a CAR, a 2A polypeptide, a nucleotide sequence encoding a foxp3 polypeptide, and a stop codon.

FIG. 5 shows flow cytometry data of the percentage of Treg and Teff cells expressing CAR after insertion of a CAR transgene into the foxp3 locus (FIG. 5A) and droplet digital PCR (ddPCR) data of the HDR efficiency of the CAR template into the Teff cells (50B.

FIG. 6 shows NHEJ editing efficiency (FIG. 6B) and FOXP3 expression (FIG. 6C) after editing with various guide sequences (FIG. 6A).

FIG. 7 shows (FIG. 7A) % FOXP3+HELIOS+ cells after editing, (FIG. 7B) FOXP3 expression after editing, and (FIG. 7C) % HDR of CAR after editing. High levels of editing were seen with HDR start, HDR START-33, and HDR START-39 guide RNAs.

FIG. 8 shows incorporation of a heterologous 3′ UTRs regulatory element at the FOXP3 STOP site. Briefly, a donor template comprising a 2A, CAR, and a heterologous 3′ UTR was inserted downstream of the FOXP3 gene in the FOXP3 locus resulting in a polynucleotide comprising a FOXP3 promoter, a start codon, a nucleic acid sequence encoding a foxp3 polypeptide, a 2A peptide, a CAR, a stop codon, and a 3′ UTR.

FIG. 9 shows CAR (FIG. 9A) and FOXP3 (FIG. 9B) expression after insertion of bGH or SV40 3′ UTR. The SV40 3′ UTR in particular results in a significant increase in FOXP3 expression as compared to the control.

FIG. 10 shows CAR (FIG. 10A) and FOXP3 (FIG. 10B) expression after editing. Specifically edits at the HDR start position or at the HDR stop position with the SV40 3′ UTR result in significant CAR and FOXP3 expression.

FIG. 11 shows that antigen-specific activation profiles are similar between engineering conditions (as described herein) despite significant differential in CAR expression. FIG. 11A shows cell proliferation measured as CFSE dilution in response of the co-incubation of cells with anti-CD3/28 dynabeads, or anti-CV CAR beads at varying ratios for the non-edited cells, HDR Start edited cells, HDR Stop W3S UTR and Lentiviral transduced (Lenti-transduced) cells. FIG. 11B shows CAR expression as measured by G4S linker presence. FIGS. 11C and 11D show percent positive CD71 and CD71 MFI, respectively, using flow cytometry.

FIG. 12 shows that gene edited effector T cells (Teff) as described herein show minimal response to CAR specific stimulation while Lenti-transduced Teff respond robustly to CAR specific stimulation. FIG. 12A shows cell proliferation of the Teff measured by CFSE dilution in response of co-incubation with no beads, anti-CD3/28 dynabeads, or anti-CV CAR beads at varying ratios for 3 days ( ). FIG. 12B shows the percent of positive CD71 cells using flow cytometry.

FIG. 13 shows that FOXP3 start edited CAR-Tregs efficiently suppress allogeneic and autologous T-responders in in-vitro suppression assays. FIGS. 13A-D show co-co-cultures of CFSE labeled Tregs or CFSE labeled Teff with CTV labeled allogeneic responder T cells (total T cells) at varying ratios of Treg/Teff and 1:10 Tresp:CTS anti CD3/28 dynabeads for 3 days. FIGS. 13A and 13B show the percent of CD4+ cell proliferation or the percent of CD8+ cell proliferation, respectively. FIGS. 13C and 13D show levels of IL-17 and IFNγ in the culture supernatants in pg/ml, respectively.

FIGS. 13E-H shows co-co-cultures of CFSE labeled Tregs or CFSE labeled Teff with CTV labeled autologous responder T cells (total T cells) at varying ratios of Treg/Teff and 1:10 Tresp:CTS anti CD3/28 dynabeads for 3 days. FIGS. 13E and 13F show the percent of CD4+ cell proliferation or percent of CD8+ cell proliferation, respectively. FIGS. 13G and 13H show levels of IL-17 and IFNγ in the culture supernatants in pg/ml, respectively.

FIG. 14 shows the percent of FOXP3+Helios+ cells measured by flow cytometry on days 0, 3, 7, 10, 14, 17, and 21 in non-edited Tregs, HDR Start edited Tregs or Lenti-transduced Tregs cultured in non-inflammatory and inflammatory cytokines conditions.

FIG. 15A shows the percent of FOXP3+ cells measured by flow cytometry on days 0, 3, 7, 10, 14, 17, and 21 in non-edited Tregs, HDR Start edited Tregs, HDR Stop edited Tregs, or Lenti-transduced Tregs cultured in non-inflammatory and inflammatory cytokines conditions and stimulated with CTS anti-CD3/CD28 dynabeads weekly. FIG. 15B shows the percent of FOXP3+ cells measured by flow cytometry on days 0, 3, 7, 10, 14, 17, and 21 in HDR Start edited Tregs, HDR Stop edited Tregs, or Lenti-transduced Tregs cultured in non-inflammatory and inflammatory cytokines conditions and stimulated with antigen specific CV beads weekly.

FIG. 16 shows that FOXP3 gene edited samples show increased resistance to pro-inflammatory culture induced destabilizing conditions. FIG. 16 shows a heatmap showing the expression of markers at the end of post-expansion protocol. Samples are divided by engineering condition (Non-edited electroporated, FOXP3 start gene editing, FOXP3_Stop gene editing, and Lentiviral vector), stimulation type (anti-CD3/28 CTS bead, CV anti-idiotype bead), and cytokine media condition (IL-2 only without proinflammatory cytokines, or IL-2 with proinflammatory cytokines). CAR positivity is gated off of live, and the remainder of markers are gated off the CAR+ fraction.

### DETAILED DESCRIPTION

### Outline

I. Compositions and Methods for Genetically Engineering Cells
   A. Autoimmune disease
   B. Regulatory T cells (Tregs)
   C. Cells comprising polynucleotides
      1. Cell types
      2. Regulatory elements
      3. Foxp3 functional derivatives
      4. Transgenes
   D. Compositions and methods for engineering cells
      1. Nucleic acid-guided nuclease systems
      2. Viral systems
II. Pharmaceutical compositions
III. Therapeutic uses
IV. Kits
V. Embodiments
VI. Examples
VII. Equivalents

### I. COMPOSITIONS AND METHODS FOR GENETICALLY ENGINEERING CELLS

Provided herein are compositions and methods for generating one or more sources of cells, genetically engineering cells, propagating and/or differentiating genetically engineered cells, using genetically engineered cells, and treating subjects suffering from autoimmune disease, such as intestinal inflammation, for example inflammatory bowel disease (IBD), e.g., Crohn's disease (CD) and ulcerative colitis (UC), with the genetically engineered cells. The genetically engineered cell can be any suitable cell type, such as a cell as disclosed herein, preferably a stem cell or an immune cell, such as an immunosuppressive immune cell, for example a regulator T cell (Treg). Particularly, provided herein are compositions and methods comprising a cell comprising a heterologous polynucleotide in an endogenous FOXP3 locus.

### A. Autoimmune Disease

Autoimmune disease is a condition arising from an abnormal immune response to a functioning body part, e.g., when a subject's own immune system mistakenly attacks healthy cells. About 24 million (~7.5%) people in the United States alone are affected by an autoimmune disease. Both humans and non-human animals suffer from autoimmune diseases.

It is contemplated that the compositions and methods disclosed herein can be used to genetically engineer cells sourced from a healthy individual and/or a subject suffering from any autoimmune disease, and the resulting genetically engineered cells can be used to treat a patient suffering from an autoimmune disease. In certain embodiments, one or more cells are isolated from a subject suffering from an autoimmune disease. In certain embodiments, the one or more cells are genetically engineered to comprise one or more transgenes. Typically, the transgene is inserted in the FOXP3 locus. In some embodiments, the transgenes are under the control of a FOXP3 promoter.

The compositions and methods provided herein can be used to treat any autoimmune disease. Exemplary autoimmune diseases include but are not limited to Acquired aplastic anemia, Acquired hemophilia, Acromegaly, Acute disseminated encephalomyelitis, Acute hemorrhagic leukoencephalitis, Adult-onset Still's disease, Agammaglobulinemia, Alopecia areata, ANCA-associated vasculitis, Ankylosing spondylitis, Anti-GBM/anti-TBM disease, Anti-NMDA receptor encephalitis, Antiphospholipid syndrome, Arteriosclerosis, Asherson's syndrome, Atopic Dermatitis, Autoimmune Addison's disease, Autoimmune autonomic ganglionopathy, Autoimmune dysautonomia, Autoimmune encephalitis, Autoimmune gastritis, Autoimmune gastrointestinal dysmotility, Autoimmune hemolytic anemia, Autoimmune hepatitis, Autoimmune hyperlipidemia, Autoimmune hypophysitis, Autoimmune inner ear disease, Autoimmune lymphoproliferative syndrome, Autoimmune myelofibrosis, Autoimmune myocarditis, Autoimmune oophoritis, Autoimmune pancreatitis, Autoimmune polyendocrine syndrome type II, Autoimmune polyglandular syndromes, types I, II, & III, Autoimmune progesterone dermatitis, Autoimmune retinopathy, Autoimmune sudden sensorineural hearing loss, Autoimmune thrombocytopenia, Autoimmune thrombocytopenia purpura, Autoimmune thyroiditis, Balo disease, Behçet's disease, Berger's disease, Birdshot chorioretinopathy, Birdshot uveitis, Bullous pemphigoid, Castleman disease, Catastrophic antiphospholipid syndrome, Celiac disease, Chagas disease, Chronic fatigue syndrome, Chronic inflammatory demyelinating polyneuropathy, Chronic Lyme disease, Chronic lymphocytic thyroiditis, Chronic urticaria, Churg-Strauss syndrome/eosinophilic granulomatosis with polyangiitis, Cogan's syndrome, Cold agglutinin disease, Complex regional pain syndrome, CREST syndrome, Crohn's disease, Cronkhite-Canada syndrome, Cryptogenic organizing pneumonia, Dermatitis herpetiformis, Dermatomyositis, Devic's disease, Diabetes (type 1), Discoid lupus, Dressler's syndrome, Eczema, Endometriosis, Eosinophilic esophagitis, Eosinophilic fasciitis, Erythema nodosum, Essential mixed cryoglobulinemia, Evans syndrome, Fibrosing alveolitis, Giant cell arteritis, Giant Cell Myocarditis, Glomerulonephritis, Goodpasture's syndrome, Granulomatosis with polyangiitis, Graves disease, Guillain-Barré syndrome, Hashimoto's thyroiditis, Hemifacial atrophy, Henoch-Schönlein purpura, Herpes gestationis, Hidradenitis suppurativa, Horton's disease, Hurst's disease, Hypogammaglobulinemia, Idiopathic pulmonary fibrosis, IgA nephropathy, IgA vasculitis, IgG4-related sclerosing disease, Immune thrombocytopenia (ITP), Immune-mediated necrotizing myopathy, Inclusion body myositis, Interstitial cystitis, Juvenile dermatomyositis, Juvenile idiopathic arthritis, Juvenile myositis, Juvenile polymyositis, Kawasaki disease, Lambert-Eaton myasthenic syndrome, Leukocytoclastic vasculitis, Lichen planus, Lichen sclerosus, Ligneous conjunctivitis, Limited cutaneous systemic sclerosis, Linear IgA bullous dermatosis, Linear IgA disease, Lupus nephritis, Lyme disease, Lymphocytic colitis, Ménière's disease, Mesenteric Panniculitis, Microscopic colitis, Microscopic polyangiitis, Mixed connective tissue disease, Mooren's ulcer, Mucha-Habermann disease, Multifocal motor neuropathy, Multiple sclerosis, Myalgic encephalomyelitis, Myasthenia gravis, Narcolepsy, Neuromyelitis optica, Ocular cicatricial pemphigoid, Opsoclonus-myoclonus syndrome, Palindromic rheumatism, Palmoplantar Pustulosis, PANS/PANDAS, Paraneoplastic cerebellar degeneration, Paraneoplastic pemphigus, Paroxysmal nocturnal hemoglobinuria, Parry-Romberg syndrome, Pars planitis, Parsonage-Turner syndrome, Pemphigus foliaceus, Pemphigus gestationis, Pemphigus vulgaris, Peripheral uveitis, Pernicious anemia, POEMS syndrome, Polyarteritis nodosa, Polymyalgia rheumatica, Polymyositis, Post-treatment Lyme disease syndrome, Postmyocardial infarction, Postpericardiotomy syndrome, Postural orthostatic tachycardia syndrome, Primary biliary cholangitis, Primary biliary cirrhosis, Primary sclerosing cholangitis, Progressive facial hemiatrophy, Psoriasis, Psoriatic arthritis, Pulmonary fibrosis, idiopathic, Pure red cell aplasia, Pyoderma gangrenosum, Raynaud's syndrome/phenomenon, Reactive arthritis, Reflex sympathetic dystrophy syndrome, Reiter's syndrome, Relapsing polychondritis, Restless leg syndrome, Rheumatic fever, Rheumatoid arthritis, Sarcoidosis, Schmidt syndrome, Scleritis, Scleroderma, Sclerosing Mesenteritis, Serpiginous choroidopathy, Sjögren's syndrome, Small fiber sensory neuropathy, Stiff person syndrome, Subacute bacterial endocarditis, Susac syndrome, Sydenham's chorea, Sympathetic ophthalmia, Systemic lupus erythematosus, Takayasu's arteritis, Temporal arteritis, Testicular autoimmunity, Thyroid eye disease, Tolosa-Hunt syndrome, Transverse myelitis, Tubulointerstitial nephritis uveitis syndrome, Ulcerative colitis, Undifferentiated connective tissue disease, Uveitis, Vasculitis, VEXAS Syndrome, Vitiligo, Vogt-Koyanagi-Harada syndrome, Wegener's granulomatosis, Willis-Ekbom disease.

Typically, treatment of autoimmune diseases depends on the type and severity of the condition. Standard treatment methods include: vitamin or hormone supplementation, blood transfusions if the disease is blood related, physical therapy if the disease impacts bones, joints, or muscles, immunosuppressant drugs to reduce the immune response against the body's own tissues, such as non-steroidal anti-inflammatory drugs (NSAIDs), glucocorticoids, and disease-modifying anti-rheumatic drugs (DMARDs). Because immunosuppressants weaken the overall immune response, relief of symptoms must be balanced with preserving the patient's ability to combat infections, which could potentially be life-threatening. Additional options include monoclonal antibodies that can be used to block pro-inflammatory cytokines, antigen-specific immunotherapy which allows immune cells to specifically target the abnormal cells that cause autoimmune disease, co-stimulatory blockade that works to block the pathway that leads to the autoimmune response, and regulatory T cell therapy that utilizes this special type of T cell to suppress the autoimmune response.

### B. Regulatory T Cells (Tregs)

The present invention provides genetically engineered Treg cells containing a transgene, e.g., a heterologous sequence, in an endogenous FOXP3 locus and/or a heterologous locus. In preferred embodiments, expression of the transgene is under the control of a FOXP3 promoter. High FOXP3 expression level is a specific phenotype for Treg cells. As a result, a transgene under the control of a FOXP3 promoter is preferentially expressed in Treg cells. Further, the transgene is expressed only as long as the cell maintains its Treg, FOXP3⁺ phenotype, e.g., reduction of FOXP3 expression results in a concomitant reduction in a transgene under the control of a FOXP3 promoter. As a result, cells genetically engineered to contain the transgene under the control of a FOXP3 promoter will have a safety feature ensuring the transgene will be preferentially expressed in Treg cells. Further, Treg cells that maintain their Treg phenotype will demonstrate preferential expression of the transgene as compared to those that lose their Treg phenotype. Once the cell loses FOXP3 gene expression, for example differentiates into an effector T cell (Teff), the transgene will no longer be expressed.

Tregs maintain immune homeostasis and confer immune tolerance. The engineered Treg cells as disclosed herein may be autologous or allogeneic, can be used in cell-based therapy to treat patients in need of induction of immune tolerance or restoration of immune homeostasis, such as patients receiving organ transplantation or allogeneic cell therapy and patients with an autoimmune diseases. The present Treg cells will have enhanced immune-regulatory activities, including improved tissue specificity and/or increased immunosuppressive functions. The Tregs will actively control the proliferation and activation of Teffs locally and/or systemically through receptor-mediated cell-to-cell contact and secretion of immunosuppressive cytokines as disclosed herein. Further, since Treg cells can proliferate and self-renew, the cells as disclosed herein can achieve long-term tolerance when used as cell therapies to treat autoimmune disease.

Tregs include CD4⁺ T cells that characteristically express the high-affinity IL-2 receptor α-chain (CD25) and master transcription factor Forkhead box P-3 (Foxp3) which is essential for their suppressive phenotype and stability. As activated CD4⁺ T cells can upregulate CD25 expression, an additional defining feature of Tregs is the absence of IL-7 receptor α-chain (CD127). Their function includes dominant controllers of self-tolerance, tissue inflammation, long-term immune homeostasis, among others. Despite making up only 5%-10% of the peripheral CD4⁺ T cell pool, Tregs exert powerful inhibitory effects on effector cells through a variety of mechanisms including cytokine secretion, metabolic disruption, inhibition of dendritic cells (DCs), and cytolysis. These mechanisms have been rigorously examined using animal models and shown to regulate autoimmune disease. For example, studies in patients with inflammatory bowel disease (IBD) have identified defects in the number and distribution of Tregs and their ability to traffic to the GI tract. Additionally, resistance to Treg-mediated suppression has been noted in lamina propria T effector cells (Teffs). Therefore, there is growing need for identification of therapeutic targets for Tregs, improvements in the generation of therapeutic Tregs, improvements in the delivery and localization of Tregs to a therapeutic site in an individual, developments in activation, proliferation, and persistence of Tregs at a therapeutic site, among others to shift the balance from an inflammatory to a more tolerogenic microenvironment in areas of active inflammation.

Tregs can be broadly divided into two groups, thymic Tregs (tTregs) or peripherally induced Tregs (pTregs), based on their developmental origin.

tTregs are generated in the thymus in the early neonatal period and migrate to peripheral organs thereafter where they maintain tolerance. Further, thymically derived CD4⁺CD25⁺ T cells possess the ability to suppress autoreactive T cells and eliminate autoimmunity; therefore, tTregs are important for maintenance of self-tolerance and prevention and/or control of autoimmune disease. pTregs are generated when naive CD4⁺CD25⁻ T cells are converted into Foxp3-expressing CD4⁺CD25⁺ Tregs by T cell receptor (TCR) co-stimulation in the presence of transforming growth factor β (TGF-β, TGFbeta). pTreg conversion in gut-associated lymphoid tissues (GALTs) is enhanced when naïve CD4⁺ T cells encounter an antigen in the presence of TGF-β, IL-2, and retinoic acid (RA). For example, pTregs are found in abundance in the intestinal lamina propria where interactions with environmental antigens can shape phenotypic differences and transcription factor expression. In patients with active IBD, the intestinal lamina propria Treg pool was significantly smaller than that of a positive control, namely patients with diverticulitis. Additionally, in these patients, the peripheral blood Treg pool was smaller as compared to inactive IBD or diverticulitis.

Tregs function as key mediators of peripheral tolerance through direct cellular contact and paracrine actions on tissues where they reside, for example, IL-10-secreting Tregs can help control inflammatory responses and selective disruption of IL-10 expression in these Tregs has been shown to cause autoimmune disease. This is one of many modalities that Tregs can employ to maintain immune homeostasis at the site of action. Others include inhibitory cytokine secretion, cytolysis of effector cells, metabolic disruption, neutralization of antigen presenting cells and promotion of tissue repair. The Treg cytokine repertoire includes the anti-inflammatory molecules IL-10, TGF-β, IL-35, PD-1, among others (as discussed herein). Tregs are capable of producing TGF-β, which profoundly suppresses the proliferation of effector T cells (Teffs). TGF-β and IL-35 can induce the generation of Tregs from naïve CD4⁺ T cells. In certain embodiments, provided herein are cells that secrete one or more cytokines, preferably an anti-inflammatory cytokine, more preferably an immune checkpoint inhibitor, even more preferably IL-10, TGF-β, and/or PD-1. In preferred embodiments, the cell further comprises one or more transgenes, for example a CAR.

The development and function of Tregs is associated with IL-2. IL-2 and the transcription factor STAT5, downstream of IL-2 alpha receptor (IL-2R alpha, i.e., CD25), induce the expression of Foxp3 and differentiation of tTregs. STAT5 activation driven by IL-2 IL-2Ralpha (CD25) and subsequent signaling enhances the suppressor function of differentiated Tregs. Further, Tregs can sequester local IL-2 reducing the available cytokine concentration for actively dividing Toffs reducing their ability to survive and proliferate. In certain embodiments, provided herein are cells comprising CD25. In certain embodiments, the cell comprises one or more CD25 bound to IL-2. In preferred embodiments, the cell further comprises one or more transgenes, for example a CAR.

Tregs can also interfere with ATP metabolism to dampen proinflammatory responses. Tregs co-express the ectoenzymes CD39 and CD73 responsible for the degradation of ATP and generation of pericellular adenosine. Adenosine stimulates the A2A receptor on Teffs exerting potent inhibitory effects. Activation of the A2A receptor also inhibits IL-6 expression while enhancing the production of TGF-β. This promotes the development of adaptive induced Tregs and simultaneously inhibits proinflammatory Th17 cell formation. In certain embodiments, provided herein are cells the co-express one or more immunosuppressive enzymes. In preferred embodiments, the cell secretes CD39 and/or CD73. In preferred embodiments, the cell further comprises one or more transgenes, for example a CAR.

The activation of T cells comprises TCR antigen/major histocompatibility complex engagement in the context of a secondary signal, namely T cell-derived CD28 binding the dendritic cell (DC) B7 ligands, CD80 and CD86. This process is negatively regulated through the production of cytotoxic T lymphocyte associated protein 4 (CTLA-4) which is constitutively expressed in Foxp3⁺ Tregs.

Tregs further can promote cell lysis of Teffs to suppress their activity. Granzyme-B expressing CD4⁺ Tregs can promote lysis of target cells in a perforin-dependent, but TCR-independent manner. Activated Tregs upregulate tumor necrosis factor-related apoptosis inducing ligand (TRAIL) which enhances CD4⁺ suppressive activity as well as cytotoxicity against T cells.

Tregs may further aid in tissue repair and maintenance distinct from their suppressive function.

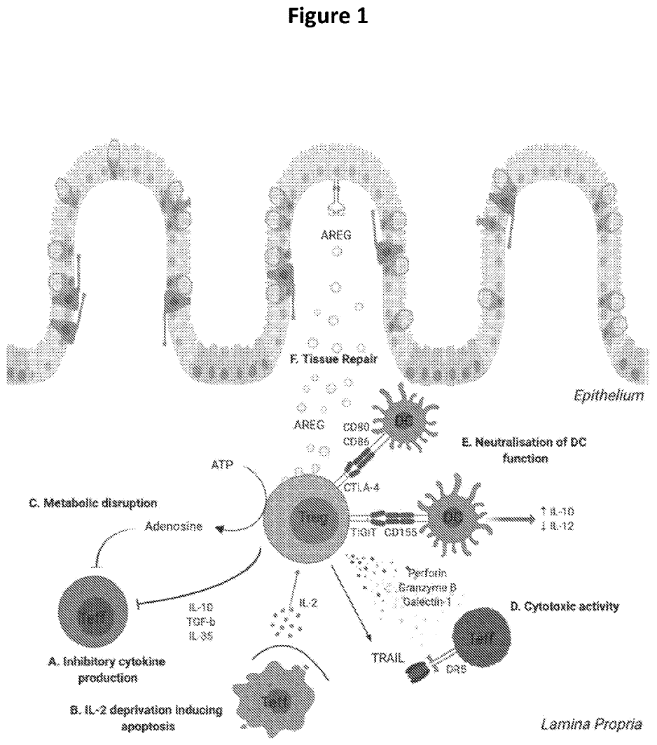

A schematic of Treg function is shown in FIG. 1. Tregs use a multitude of mechanisms to promote a tolerogenic microenvironment and tissue repair. (A) Secretion of the anti-inflammatory cytokines, IL-10, TGF-β and IL-35, not only inhibit Teff proliferation but also suppress Th1 and Th17 effector function, both of which are key mediators of IBD. (B) Tregs express the high-affinity IL-2 receptor α-chain (CD25) consuming local IL-2 with greater affinity than effector cells. Teffs which are ‘starved’ of IL-2 exhibit restricted proliferation and undergo apoptosis. (C) Tregs co-expressing CD39 and CD73 disrupt metabolic processes in effector cells by converting ATP into pericellular adenosine, a potent inhibitor of Teff function. Additionally, adenosine stimulates TGF-β production, promoting development of pTregs. (D) Tregs are capable of secreting perforin, granzyme B and galectin-1 which are directly cytotoxic against Teffs. Activated Tregs also express TRAIL, inducing apoptosis of Teffs through the TRAIL/DR5 pathway. (E) Expression of CTLA-4 degrades DC-derived CD80 and CD86 leading to impaired CD28-mediated co-stimulation of T cells. DC function is further inhibited through the interaction of Treg-derived TIGIT and CD155 on DCs. This induces IL-10 production and suppresses IL-12. (F) In response to alarmins, Tregs produce AREG, an important regulator of tissue repair and regeneration. AREG, amphiregulin; CTLA-4, cytotoxic T lymphocyte associated protein 4; DC, dendritic cell; DR5, death receptor 5; IL, interleukin; Th1, T helper 1 cell; Th17, T helper 17 cell; TIGIT, T-cell immunoreceptor with Ig and immunoreceptor tyrosine-based inhibitory motif domains.

### C. Cell Comprising Polynucleotides

### 1. Cells Comprising Polynucleotides in a FOXP3 Locus

Contemplated herein are cells comprising a polynucleotide in a FOXP3 locus. The polynucleotide can comprise any suitable nucleotide sequence. In certain embodiments, the polynucleotide comprises one or more of (1) a regulatory element, (2) a FOXP3 gene or a functional derivative thereof, (3) a transgene, (4) a self-cleaving peptide, and (5) a heterologous untranslated region.

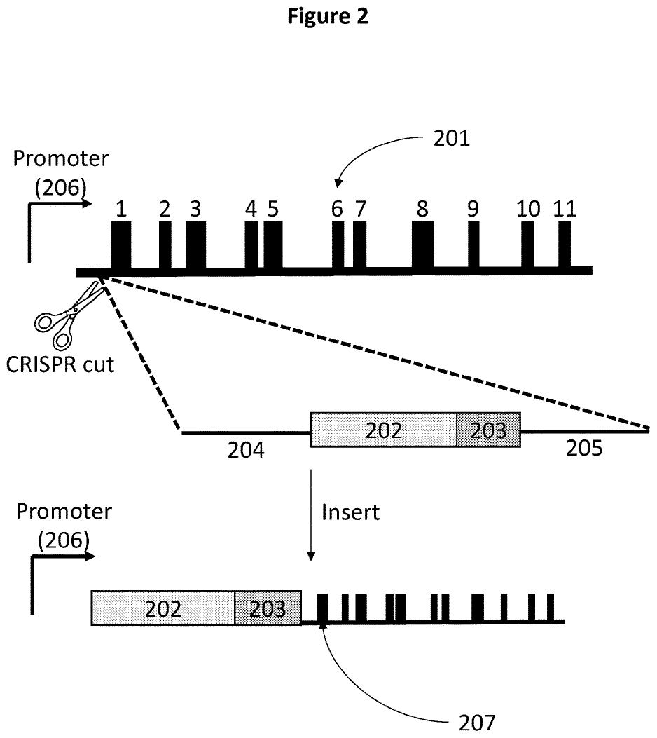

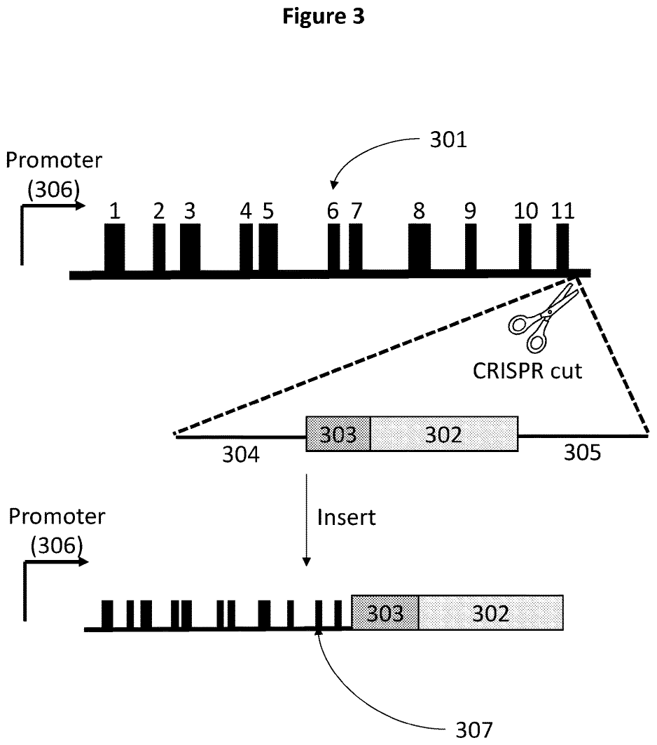

In certain embodiments, provided herein is a cell comprising a polynucleotide in a FOXP3 locus comprising a nucleotide sequence comprising a promoter, a FOXP3 gene or a functional derivative thereof, and a transgene, wherein expression of the FOXP3 gene or functional derivative thereof and the transgene are under the control of the promoter. In certain embodiments, the nucleotide sequence further comprises a heterologous 3′ untranslated region (UTR). The promoter can be any suitable promoter, such as a promoter as disclosed herein. In certain embodiments, the promoter comprises a FOXP3 promoter. The transgene can be inserted into any suitable position in the FOXP3 locus, for example upstream or downstream of the FOXP3 gene. In certain embodiments, the transgene is inserted upstream of the FOXP3 gene, preferably at least 1, 2, 3, 6, 9, 12, 15, 30, 60, 61, 90, 120, 240, or 480 bases and/or not more than 1920, 960, 480, 240, 120, or 90 bases upstream of the FOXP3 gene, more preferably about 61 to 1 bases before the start codon of the FOXP3 gene. In certain embodiments, the transgene is inserted immediately upstream of the start codon of the FOXP3 gene. In certain embodiments, the transgene is inserted downstream of the FOXP3 gene, preferably at least 1, 2, 3, 6, 9, 12, 15, 30, 60, 61, 90, 120, 240, or 480 bases and/or not more than 1920, 960, 480, 240, 120, or 90 bases downstream of the FOXP3 gene, more preferably about 61 to 1 bases after the stop codon of the FOXP3 gene. In certain embodiments, the transgene is inserted immediately after the stop codon of the FOXP3 gene. In this case, the stop codon is typically engineered and/or modified to recode the stop codon to an amino acid encoding codon. In other embodiments, the transgene is inserted immediately before the stop codon of the FOXP3 gene. The polypeptide encoded in by the polynucleotide is preferably a foxp3 polypeptide. In other words, the polynucleotide in the FOXP3 locus encodes a full length foxp3 polypeptide. In certain embodiments, the nucleotide sequence further encodes a self-cleaving peptide (SCP). Typically the nucleotide sequence encoding the SCP is between the FOXP3 gene and the transgene, such that the nucleotide sequence encodes a single polypeptide comprising the foxp3 polypeptide, the self-cleaving polypeptide, and the transgene. Any suitable self-cleaving peptide can be used, such as a SCP as disclosed herein, preferably a 2A peptide, more preferably a P2A, E2A, F2A, or T2A peptide. In preferred embodiments, the FOXP3 gene and the transgene are expressed as a single polypeptide. The FOXP3 gene and the transgene can be constructed in any suitable orientation. In certain embodiments, the nucleotide sequence encodes a polypeptide comprising from N- to C-terminus the following polypeptide: transgene-SCP-foxp3. In certain embodiments, the nucleotide sequence encodes a polypeptide comprising from N- to C-terminus the following polypeptide: foxp3-SCP-transgene. In certain embodiments, the cell further comprises the foxp3 polypeptide encoded by the nucleotide sequence and/or the cleaved derivative thereof. In certain embodiments, the cell comprising a foxp3 polypeptide comprising a first portion of the self-cleaving peptide and a polypeptide encoded by the transgene comprising a second portion of the self-cleaving peptide. In certain embodiments, the transgene is expressed intracellularly, secreted, and/or displayed on the surface of the cell. In certain embodiments, the FOXP3 gene and the transgene are expressed as separate polypeptides. Typically in this case the FOXP3 and the transgene are present in separate open reading frames. The two genes can have different regulatory elements such that they are expressed as two different mRNAs and then subsequently translated. Alternatively, the two genes can be expressed as a single mRNA comprising a ribosome binding element between the two genes, such as an internal ribosome entry site (IRES). Any suitable ribosome binding element can be used and a skilled artisan would understand how to select the correct element. The transgene (TG) can encode any suitable gene or genes, for example a cytokine, an interleukin, a cytokine receptor, a transcription factor, a chimeric receptor, a reporter gene, a selectable marker, a transgene as disclosed herein, or a combination thereof. In certain embodiments, the transgene encodes a chimeric antigen receptor (CAR) or CAR component. In certain embodiments, the transgene encodes a T-cell receptor (TCR). In certain embodiments, the transgene encodes a chemokine receptor (CR) as disclosed herein, such as a CCR or a CXCR receptor, for example CCR7 and/or CCR9. In certain embodiments, the nucleotide sequence encodes a polypeptide comprising from N- to C-terminus: TG-SCP-foxp3 (such as FIG. 2, 207). In certain embodiments, the nucleotide sequence encodes a polypeptide comprising from N- to C-terminus: CAR-SCP₁-TG-SCP₂-foxp3. In certain embodiments, the nucleotide sequence encodes a polypeptide comprising from N- to C-terminus: CAR-SCP₁-CR-SCP₂-foxp3. In certain embodiments, the nucleotide sequence encodes a polypeptide comprising from N- to C-terminus: TG-SCP₁-CAR-SCP₂-foxp3. In certain embodiments, the nucleotide sequence encodes a polypeptide comprising from N- to C-terminus: CR-SCP₁ CAR-SCP₂-foxp3. SCP₁ and SCP₂ can be the same or different. In preferred embodiments, SCP₁ and SCP₂ are different. In certain embodiments, the nucleotide sequence encodes a polypeptide comprising from N- to C-terminus: foxp3-SCP-TG (such as FIG. 3, 307). In certain embodiments, the nucleotide sequence encodes a polypeptide comprising from N- to C-terminus: foxp3-SCP₁-CAR-SCP₂-TG. In certain embodiments, the nucleotide sequence encodes a polypeptide comprising from N- to C-terminus: foxp3-SCP₁-CAR-SCP₂-CR. In certain embodiments, the nucleotide sequence encodes a polypeptide comprising from N- to C-terminus: foxp3-SCP₁-TG-SCP₂-CAR. In certain embodiments, the nucleotide sequence encodes a polypeptide comprising from N- to C-terminus: foxp3-SCP₁-CR-SCP₂-CAR. SCP₁ and SCP₂ can be the same or different. In preferred embodiments, SCP₁ and SCP₂ are different. In certain embodiments, the nucleotide sequence comprises a UTR. Any suitable UTR may be used. In certain embodiments, the nucleotide sequence comprises a heterologous 3′ UTR. Any suitable heterologous 3′ UTR can be used, for example a SV40 reversed, bGH, WPRE, WPRE3/SV40p128, SV40 corrected, WPRE3/SV40.2, TCRA EIF3, or s27a3RU, preferably a SV40 reversed, SV40 corrected, or bGH 3′ UTR. In certain embodiments, the FOXP3 gene further comprises one or more mutations in a codon encoding a ubiquitination or deubiquitination site, whereby the resulting functional derivative demonstrates increased stability as compared to the wild-type foxp3 polypeptide. In certain embodiments, the ubiquitination or deubiquitination site comprises codon 227, 250, 263, 268, and/or 422. The cell can be any suitable cell, such as a cell as disclosed herein or a derivative of a cell as disclosed herein. In certain embodiments, the cell comprises a eukaryotic cell, preferably a metazoan cell, more preferably a human cell. In certain embodiments, the cell comprises an immune cell, such as a neutrophil, eosinophil, basophil, mast cell, monocyte, macrophage, dendritic cell, natural killer cell, T cell, or a lymphocyte, or an immune precursor cell, such as a stem cell, an induced pluripotent stem cell, a hematopoietic stem cell, or a CD4⁺ stem cell. In certain embodiments, the immune cell comprises a T cell, preferably an immunosuppressive T cell, more preferably a Treg. In certain embodiments, the cell is isolated from an individual, such as an individual suffering from a disease or a healthy individual. The terms “individual” and “subject” may be used interchangeably. In certain embodiments, the disease comprises a disease as disclosed herein, preferably an autoimmune disease, such as intestinal inflammation, for example IBD, CD, or UC. In certain embodiments, the cell is further engineered to not promoter an immune response in an allogeneic individual as disclosed herein.

In certain embodiments, provided herein is a cell comprising a polynucleotide in a FOXP3 locus comprising a nucleotide sequence comprising a promoter and a transgene encoding a chimeric antigen receptor (CAR) and a chemokine receptor (CR), wherein expression of the transgene is under the control of the promoter. In certain embodiments, the nucleotide sequence further comprises a heterologous 3′ UTR. In certain embodiments, the promoter comprises a FOXP3 promoter. In certain embodiments, the nucleotide sequence further comprises a FOXP3 gene or a functional derivative thereof, wherein expression of the FOXP3 gene or functional derivative thereof is under the control of the FOXP3 promoter. In certain embodiments, the FOXP3 gene encodes a full length foxp3 polypeptide. The promoter can be any suitable promoter, such as a promoter as disclosed herein. Typically, the promoter comprises a FOXP3 promoter. The transgene can be inserted into any suitable position in the FOXP3 locus, for example upstream or downstream of the FOXP3 gene. In certain embodiments, the transgene is inserted upstream of the FOXP3 gene, preferably at least 1, 2, 3, 6, 9, 12, 15, 30, 60, 61, 90, 120, 240, or 480 bases and/or not more than 1920, 960, 480, 240, 120, or 90 bases upstream of the FOXP3 gene, more preferably about 61 to 1 bases before the start codon of the FOXP3 gene. In certain embodiments, the transgene is inserted immediately upstream of the start codon of the FOXP3 gene. In certain embodiments, the transgene is inserted downstream of the FOXP3 gene, preferably at least 1, 2, 3, 6, 9, 12, 15, 30, 60, 61, 90, 120, 240, or 480 bases and/or not more than 1920, 960, 480, 240, 120, or 90 bases downstream of the FOXP3 gene, more preferably about 61 to 1 bases after the stop codon of the FOXP3 gene. In certain embodiments, the transgene is inserted immediately after the stop codon of the FOXP3 gene. In this case, the stop codon is typically engineered and/or modified to recode the stop codon to an amino acid encoding codon. In other embodiments, the transgene is inserted immediately before the stop codon of the FOXP3 gene. The polypeptide encoded in by the polynucleotide is preferably a foxp3 polypeptide. In other words, the polynucleotide in the FOXP3 locus encodes a full length foxp3 polypeptide. In certain embodiments, the nucleotide sequence further encodes a self-cleaving peptide (SCP). Typically the nucleotide sequence encoding the SCP is between the FOXP3 gene and the transgene, such that the nucleotide sequence encodes a single polypeptide comprising the foxp3 polypeptide, the self-cleaving polypeptide, and the transgene. Any suitable self-cleaving peptide can be used, such as a SCP as disclosed herein, preferably a 2A peptide, more preferably a P2A, E2A, F2A, or T2A peptide. In preferred embodiments, the FOXP3 gene and the transgene are expressed as a single polypeptide. The FOXP3 gene and the transgene can be constructed in any suitable orientation. In certain embodiments, the nucleotide sequence encodes a polypeptide comprising from N- to C-terminus the following polypeptide: transgene-SCP-foxp3. In certain embodiments, the nucleotide sequence encodes a polypeptide comprising from N- to C-terminus the following polypeptide: foxp3-SCP-transgene. In certain embodiments, the cell further comprises the foxp3 polypeptide encoded by the nucleotide sequence and/or the cleaved derivative thereof. In certain embodiments, the cell comprising a foxp3 polypeptide comprising a first portion of the self-cleaving peptide and a polypeptide encoded by the transgene comprising a second portion of the self-cleaving peptide. In certain embodiments, the transgene is expressed intracellularly, secreted, and/or displayed on the surface of the cell. In certain embodiments, the FOXP3 gene and the transgene are expressed as separate polypeptides. Typically in this case the FOXP3 and the transgene are present in separate open reading frames. The two genes can have different regulatory elements such that they are expressed as two different mRNAs and then subsequently translated. Alternatively, the two genes can be expressed as a single mRNA comprising a ribosome binding element between the two genes, such as an internal ribosome entry site (IRES). Any suitable ribosome binding element can be used and a skilled artisan would understand how to select the correct element. The transgene (TG) can encode any suitable gene or genes, for example a cytokine, an interleukin, a cytokine receptor, a transcription factor, a chimeric receptor, a reporter gene, a selectable marker, a transgene as disclosed herein, or a combination thereof. In certain embodiments, the transgene encodes a chimeric antigen receptor (CAR) or CAR component. In certain embodiments, the transgene encodes a T-cell receptor (TCR), In certain embodiments, the transgene encodes a chemokine receptor (CR) as disclosed herein, such as a CCR or a CXCR receptor, for example CCR7 and/or CCR9. In certain embodiments, the nucleotide sequence encodes a polypeptide comprising from N- to C-terminus: TG-SCP-foxp3 (such as FIG. 2, 207). In certain embodiments, the nucleotide sequence encodes a polypeptide comprising from N- to C-terminus: CAR-SCP₁-TG-SCP₂-foxp3. In certain embodiments, the nucleotide sequence encodes a polypeptide comprising from N- to C-terminus: CAR-SCP₁-CR-SCP₂-foxp3. In certain embodiments, the nucleotide sequence encodes a polypeptide comprising from N- to C-terminus: TG-SCP₁-CAR-SCP₂-foxp3. In certain embodiments, the nucleotide sequence encodes a polypeptide comprising from N- to C-terminus: CR-SCP₁CAR-SCP₂-foxp3. SCP₁ and SCP₂ can be the same or different. In preferred embodiments, SCP₁ and SCP₂ are different. In certain embodiments, the nucleotide sequence encodes a polypeptide comprising from N- to C-terminus: foxp3-SCP-TG (such as FIG. 3, 307). In certain embodiments, the nucleotide sequence encodes a polypeptide comprising from N- to C-terminus: foxp3-SCP1-CAR-SCP2-TG. In certain embodiments, the nucleotide sequence encodes a polypeptide comprising from N- to C-terminus: foxp3-SCP1-CAR-SCP2-CR. In certain embodiments, the nucleotide sequence encodes a polypeptide comprising from N- to C-terminus: foxp3-SCP1-TG-SCP2-CAR. In certain embodiments, the nucleotide sequence encodes a polypeptide comprising from N- to C-terminus: foxp3-SCP1-CR-SCP2-CAR. SCP1 and SCP2 can be the same or different. In preferred embodiments, SCP1 and SCP2 are different. Any suitable UTR can be used. In certain embodiments, the nucleotide sequence comprises a heterologous 3′ UTR. Any suitable heterologous 3′ UTR can be used, for example a SV40 reversed, bGH, WPRE, WPRE3/SV40p128, SV40 corrected, WPRE3/SV40.2, TCRA EIF3, or s27a3RU, preferably a SV40 reversed, SV40 corrected, or bGH 3′ UTR. In certain embodiments, the FOXP3 gene further comprises one or more mutations in a codon encoding a ubiquitination or deubiquitination site, whereby the resulting functional derivative demonstrates increased stability as compared to the wild-type foxp3 polypeptide. In certain embodiments, the ubiquitination or deubiquitination site comprises codon 227, 250, 263, 268, and/or 422. The cell can be any suitable cell, such as a cell as disclosed herein or a derivative of a cell as disclosed herein. In certain embodiments, the cell comprises a eukaryotic cell, preferably a metazoan cell, more preferably a human cell. In certain embodiments, the cell comprises an immune cell, such as a neutrophil, eosinophil, basophil, mast cell, monocyte, macrophage, dendritic cell, natural killer cell, T cell, or a lymphocyte, or an immune precursor cell, such as a stem cell, an induced pluripotent stem cell, a hematopoietic stem cell, or a CD4⁺ stem cell. In certain embodiments, the immune cell comprises a T cell, preferably an immunosuppressive T cell, more preferably a Treg. In certain embodiments, the cell is isolated from an individual, such as an individual suffering from a disease or a health individual. In certain embodiments, the disease comprises a disease as disclosed herein, preferably an autoimmune disease, such as intestinal inflammation, for example IBD, CD, or UC. In certain embodiments, the cell is further engineered to not promoter an immune response in an allogeneic individual as disclosed herein.

In certain embodiments, provided herein as cell comprising a polynucleotide in a FOXP3 locus comprising a nucleotide sequence comprising a promoter, a FOXP3 gene or a functional derivative thereof, and a heterologous 3′ UTR, wherein expression of the FOXP3 gene or functional derivative thereof is under the control of the promoter. The promoter can be any suitable promoter, such as a promoter as disclosed herein. In certain embodiments, the promoter comprises a FOXP3 promoter. The transgene can be inserted into any suitable position in the FOXP3 locus, for example upstream or downstream of the FOXP3 gene. In certain embodiments, the transgene is inserted upstream of the FOXP3 gene, preferably at least 1, 2, 3, 6, 9, 12, 15, 30, 60, 61, 90, 120, 240, or 480 bases and/or not more than 1920, 960, 480, 240, 120, or 90 bases upstream of the FOXP3 gene, more preferably about 61 to 1 bases before the start codon of the FOXP3 gene. In certain embodiments, the transgene is inserted immediately upstream of the start codon of the FOXP3 gene. In certain embodiments, the transgene is inserted downstream of the FOXP3 gene, preferably at least 1, 2, 3, 6, 9, 12, 15, 30, 60, 61, 90, 120, 240, or 480 bases and/or not more than 1920, 960, 480, 240, 120, or 90 bases downstream of the FOXP3 gene, more preferably about 61 to 1 bases after the stop codon of the FOXP3 gene. In certain embodiments, the transgene is inserted immediately after the stop codon of the FOXP3 gene. In this case, the stop codon is typically engineered and/or modified to recode the stop codon to an amino acid encoding codon. In other embodiments, the transgene is inserted immediately before the stop codon of the FOXP3 gene. The polypeptide encoded in by the polynucleotide is preferably a foxp3 polypeptide. In other words, the polynucleotide in the FOXP3 locus encodes a full length foxp3 polypeptide. In certain embodiments, the nucleotide sequence further encodes a self-cleaving peptide (SCP). Typically the nucleotide sequence encoding the SCP is between the FOXP3 gene and the transgene, such that the nucleotide sequence encodes a single polypeptide comprising the foxp3 polypeptide, the self-cleaving polypeptide, and the transgene. Any suitable self-cleaving peptide can be used, such as a SCP as disclosed herein, preferably a 2A peptide, more preferably a P2A, E2A, F2A, or T2A peptide. In preferred embodiments, the FOXP3 gene and the transgene are expressed as a single polypeptide. The FOXP3 gene and the transgene can be constructed in any suitable orientation. In certain embodiments, the nucleotide sequence encodes a polypeptide comprising from N- to C-terminus the following polypeptide: transgene-SCP-foxp3. In certain embodiments, the nucleotide sequence encodes a polypeptide comprising from N- to C-terminus the following polypeptide: foxp3-SCP-transgene. In certain embodiments, the cell further comprises the foxp3 polypeptide encoded by the nucleotide sequence and/or the cleaved derivative thereof. In certain embodiments, the cell comprising a foxp3 polypeptide comprising a first portion of the self-cleaving peptide and a polypeptide encoded by the transgene comprising a second portion of the self-cleaving peptide. In certain embodiments, the transgene is expressed intracellularly, secreted, and/or displayed on the surface of the cell. In certain embodiments, the FOXP3 gene and the transgene are expressed as separate polypeptides. Typically in this case the FOXP3 and the transgene are present in separate open reading frames. The two genes can have different regulatory elements such that they are expressed as two different mRNAs and then subsequently translated. Alternatively, the two genes can be expressed as a single mRNA comprising a ribosome binding element between the two genes, such as an internal ribosome entry site (IRES). Any suitable ribosome binding element can be used and a skilled artisan would understand how to select the correct element. The transgene (TG) can encode any suitable gene or genes, for example a cytokine, an interleukin, a cytokine receptor, a transcription factor, a chimeric receptor, a reporter gene, a selectable marker, a transgene as disclosed herein, or a combination thereof. In certain embodiments, the transgene encodes a chimeric antigen receptor (CAR) or CAR component. In certain embodiments, the transgene encodes a T-cell receptor (TCR), In certain embodiments, the transgene encodes a chemokine receptor (CR) as disclosed herein, such as a CCR or a CXCR receptor, for example CCR7 and/or CCR9. In certain embodiments, the nucleotide sequence encodes a polypeptide comprising from N- to C-terminus: TG-SCP-foxp3 (such as FIG. 2, 207). In certain embodiments, the nucleotide sequence encodes a polypeptide comprising from N- to C-terminus: CAR-SCP₁-TG-SCP₂-foxp3. In certain embodiments, the nucleotide sequence encodes a polypeptide comprising from N- to C-terminus: CAR-SCP₁-CR-SCP₂-foxp3. In certain embodiments, the nucleotide sequence encodes a polypeptide comprising from N- to C-terminus: TG-SCP₁-CAR-SCP₂-foxp3. In certain embodiments, the nucleotide sequence encodes a polypeptide comprising from N- to C-terminus: CR-SCP₁ CAR-SCP₂-foxp3. SCP; and SCP2 can be the same or different. In preferred embodiments, SCP₁ and SCP₂ are different. In certain embodiments, the nucleotide sequence encodes a polypeptide comprising from N- to C-terminus: foxp3-SCP-TG (such as FIG. 3, 307). In certain embodiments, the nucleotide sequence encodes a polypeptide comprising from N- to C-terminus: foxp3-SCP1-CAR-SCP2-TG. In certain embodiments, the nucleotide sequence encodes a polypeptide comprising from N- to C-terminus: foxp3-SCP1-CAR-SCP2-CR. In certain embodiments, the nucleotide sequence encodes a polypeptide comprising from N- to C-terminus: foxp3-SCP1-TG-SCP2-CAR. In certain embodiments, the nucleotide sequence encodes a polypeptide comprising from N- to C-terminus: foxp3-SCP1-CR-SCP2-CAR. SCP1 and SCP2 can be the same or different. In preferred embodiments, SCP1 and SCP2 are different. Any suitable UTR can be used. In certain embodiments, the nucleotide sequence comprises a heterologous 3′ UTR. Any suitable heterologous 3′ UTR can be used, for example a SV40 reversed, bGH, WPRE, WPRE3/SV40p128, SV40 corrected, WPRE3/SV40.2, TCRA EIF3, or s27a3RU, preferably a SV40 reversed, SV40 corrected, or bGH 3′ UTR. In certain embodiments, the FOXP3 gene further comprises one or more mutations in a codon encoding a ubiquitination or deubiquitination site, whereby the resulting functional derivative demonstrates increased stability as compared to the wild-type foxp3 polypeptide. In certain embodiments, the ubiquitination or deubiquitination site comprises codon 227, 250, 263, 268, and/or 422. The cell can be any suitable cell, such as a cell as disclosed herein or a derivative of a cell as disclosed herein. In certain embodiments, the cell comprises a eukaryotic cell, preferably a metazoan cell, more preferably a human cell. In certain embodiments, the cell comprises an immune cell, such as a neutrophil, eosinophil, basophil, mast cell, monocyte, macrophage, dendritic cell, natural killer cell, T cell, or a lymphocyte, or an immune precursor cell, such as a stem cell, an induced pluripotent stem cell, a hematopoietic stem cell, or a CD4⁺ stem cell. In certain embodiments, the immune cell comprises a T cell, preferably an immunosuppressive T cell, more preferably a Treg. In certain embodiments, the cell is isolated from an individual, such as an individual suffering from a disease or a health individual. In certain embodiments, the disease comprises a disease as disclosed herein, preferably an autoimmune disease, such as intestinal inflammation, for example IBD, such as CD, or UC. In certain embodiments, the cell is further engineered to not promote an immune response in an allogeneic individual as disclosed herein.

In certain embodiments, provided herein is a cell comprising a polynucleotide in a FOXP3 locus comprising a nucleotide sequence comprising a FOXP3 gene or a functional derivative thereof and a heterologous 5′ and/or 3′ UTR, wherein expression of the FOXP3 or functional derivative thereof is under the control of the promoter. In certain embodiments, the FOXP3 gene encodes a full length foxp3 polypeptide. In certain embodiments, the nucleotide sequence further comprises a transgene, wherein expression of the transgene in under the control of the promoter. In certain embodiments, the promoter comprises a FOXP3 promoter. In certain embodiments the transgene encodes a cytokine, interleukin, cytokine receptor, transcription factor, and/or chimeric receptor. In certain embodiments, the transgene encodes a chimeric antigen receptor (CAR) or CAR component. In certain embodiments, the transgene encodes a chemokine receptor. In certain embodiments, the transgene encodes a self-cleaving peptide. The promoter can be any suitable promoter, such as a promoter as disclosed herein. In certain embodiments, the promoter comprises a FOXP3 promoter. The transgene can be inserted into any suitable position in the FOXP3 locus, for example upstream or downstream of the FOXP3 gene. In certain embodiments, the transgene is inserted upstream of the FOXP3 gene, preferably at least 1, 2, 3, 6, 9, 12, 15, 30, 60, 61, 90, 120, 240, or 480 bases and/or not more than 1920, 960, 480, 240, 120, or 90 bases upstream of the FOXP3 gene, more preferably about 61 to 1 bases before the start codon of the FOXP3 gene. In certain embodiments, the transgene is inserted immediately upstream of the start codon of the FOXP3 gene. In certain embodiments, the transgene is inserted downstream of the FOXP3 gene, preferably at least 1, 2, 3, 6, 9, 12, 15, 30, 60, 61, 90, 120, 240, or 480 bases and/or not more than 1920, 960, 480, 240, 120, or 90 bases downstream of the FOXP3 gene, more preferably about 61 to 1 bases after the stop codon of the FOXP3 gene. In certain embodiments, the transgene is inserted immediately after the stop codon of the FOXP3 gene. In this case, the stop codon is typically engineered and/or modified to recode the stop codon to an amino acid encoding codon. In other embodiments, the transgene is inserted immediately before the stop codon of the FOXP3 gene. The polypeptide encoded in by the polynucleotide is preferably a foxp3 polypeptide. In other words, the polynucleotide in the FOXP3 locus encodes a full length foxp3 polypeptide. In certain embodiments, the nucleotide sequence further encodes a self-cleaving peptide (SCP). Typically the nucleotide sequence encoding the SCP is between the FOXP3 gene and the transgene, such that the nucleotide sequence encodes a single polypeptide comprising the foxp3 polypeptide, the self-cleaving polypeptide, and the transgene. Any suitable self-cleaving peptide can be used, such as a SCP as disclosed herein, preferably a 2A peptide, more preferably a P2A, E2A, F2A, or T2A peptide. In preferred embodiments, the FOXP3 gene and the transgene are expressed as a single polypeptide. The FOXP3 gene and the transgene can be constructed in any suitable orientation. In certain embodiments, the nucleotide sequence encodes a polypeptide comprising from N- to C-terminus the following polypeptide: transgene-SCP-foxp3. In certain embodiments, the nucleotide sequence encodes a polypeptide comprising from N- to C-terminus the following polypeptide: foxp3-SCP-transgene. In certain embodiments, the cell further comprises the foxp3 polypeptide encoded by the nucleotide sequence and/or the cleaved derivative thereof. In certain embodiments, the cell comprising a foxp3 polypeptide comprising a first portion of the self-cleaving peptide and a polypeptide encoded by the transgene comprising a second portion of the self-cleaving peptide. In certain embodiments, the transgene is expressed intracellularly, secreted, and/or displayed on the surface of the cell. In certain embodiments, the FOXP3 gene and the transgene are expressed as separate polypeptides. Typically in this case the FOXP3 and the transgene are present in separate open reading frames. The two genes can have different regulatory elements such that they are expressed as two different mRNAs and then subsequently translated. Alternatively, the two genes can be expressed as a single mRNA comprising a ribosome binding element between the two genes, such as an internal ribosome entry site (IRES). Any suitable ribosome binding element can be used and a skilled artisan would understand how to select the correct element. The transgene (TG) can encode any suitable gene or genes, for example a cytokine, an interleukin, a cytokine receptor, a transcription factor, a chimeric receptor, a reporter gene, a selectable marker, a transgene as disclosed herein, or a combination thereof. In certain embodiments, the transgene encodes a chimeric antigen receptor (CAR) or CAR component. In certain embodiments, the transgene encodes a T-cell receptor (TCR). In certain embodiments, the transgene encodes a chemokine receptor (CR) as disclosed herein, such as a CCR or a CXCR receptor, for example CCR7 and/or CCR9. In certain embodiments, the nucleotide sequence encodes a polypeptide comprising from N- to C-terminus: TG-SCP-foxp3 (such as FIG. 2, 207). In certain embodiments, the nucleotide sequence encodes a polypeptide comprising from N- to C-terminus: CAR-SCP₁-TG-SCP₂-foxp3. In certain embodiments, the nucleotide sequence encodes a polypeptide comprising from N- to C-terminus: CAR-SCP₁-CR-SCP₂-foxp3. In certain embodiments, the nucleotide sequence encodes a polypeptide comprising from N- to C-terminus: TG-SCP₁-CAR-SCP₂-foxp3. In certain embodiments, the nucleotide sequence encodes a polypeptide comprising from N- to C-terminus: CR-SCP₁ CAR-SCP₂-foxp3. SCP1 and SCP2 can be the same or different. In preferred embodiments, SCP₁ and SCP₂ are different. In certain embodiments, the nucleotide sequence encodes a polypeptide comprising from N- to C-terminus: foxp3-SCP-TG (such as FIG. 3, 307). In certain embodiments, the nucleotide sequence encodes a polypeptide comprising from N- to C-terminus: foxp3-SCP1-CAR-SCP2-TG. In certain embodiments, the nucleotide sequence encodes a polypeptide comprising from N- to C-terminus: foxp3-SCP1-CAR-SCP2-CR. In certain embodiments, the nucleotide sequence encodes a polypeptide comprising from N- to C-terminus: foxp3-SCP1-TG-SCP2-CAR. In certain embodiments, the nucleotide sequence encodes a polypeptide comprising from N- to C-terminus: foxp3-SCP1-CR-SCP2-CAR. SCP1 and SCP2 can be the same or different. In preferred embodiments, SCP1 and SCP2 are different. Any suitable UTR can be used. In certain embodiments, the nucleotide sequence comprises a heterologous 3′ UTR. Any suitable heterologous 3′ UTR can be used, for example a SV40 reversed, bGH, WPRE, WPRE3/SV40p128, SV40 corrected, WPRE3/SV40.2, TCRA EIF3, or s27a3RU, preferably a SV40 reversed, SV40 corrected, or bGH 3′ UTR. In certain embodiments, the FOXP3 gene further comprises one or more mutations in a codon encoding a ubiquitination or deubiquitination site, whereby the resulting functional derivative demonstrates increased stability as compared to the wild-type foxp3 polypeptide. In certain embodiments, the ubiquitination or deubiquitination site comprises codon 227, 250, 263, 268, and/or 422. The cell can be any suitable cell, such as a cell as disclosed herein or a derivative of a cell as disclosed herein. In certain embodiments, the cell comprises a eukaryotic cell, preferably a metazoan cell, more preferably a human cell. In certain embodiments, the cell comprises an immune cell, such as a neutrophil, eosinophil, basophil, mast cell, monocyte, macrophage, dendritic cell, natural killer cell, T cell, or a lymphocyte, or an immune precursor cell, such as a stem cell, an induced pluripotent stem cell, a hematopoietic stem cell, or a CD4⁺ stem cell. In certain embodiments, the immune cell comprises a T cell, preferably an immunosuppressive T cell, more preferably a Treg. In certain embodiments, the cell is isolated from an individual, such as an individual suffering from a disease or a health individual. In certain embodiments, the disease comprises a disease as disclosed herein, preferably an autoimmune disease, such as intestinal inflammation, for example IBD, such as CD, or UC. In certain embodiments, the cell is further engineered to not promote an immune response in an allogeneic individual as disclosed herein.

In certain embodiments, provided herein is a derivative of or a progeny of a cell as disclosed herein. As used herein, the term “derivative” includes any cell that derived from a cell as disclosed herein, for example a cell differentiated from a cell as disclosed herein, or a cell comprising additional genetic modifications wherein the parental cell was a cell as disclosed herein. As used herein, the term “progeny” includes any cell that was propagated from a cell as disclosed herein.

In certain embodiments, provided herein are compositions comprising one or more cells as disclosed herein.

### 2. Cell Types

Any suitable cell can be used in the disclosed compositions and methods, for example a eukaryotic cell, preferably a metazoan cell, more preferably a human cell, even preferably a stem cell or an immune cell. In certain embodiments, the cell comprises an immune cell comprising a neutrophil, eosinophil, basophil, mast cell, monocyte, macrophage, dendritic cell, natural killer cell, T cell, or a lymphocyte. In preferred embodiments, the cell comprises an immune cell comprising a T cell, preferably an immunosuppressive T cell, more preferably a Treg. In preferred embodiments, the cell comprises a Treg cell, more preferably a Treg cell that is Foxp3⁺, CD25⁺, HELIOS⁺, and/or CD127low, even more preferably Foxp3⁺, CD25⁺, HELIOS⁺, and CD127low. It is to be understood that any suitable immunosuppressive T cell can be used, for example a Treg cell and/or a Tr1 cell. In certain embodiments, the cell is CD71⁺, i.e., the cell comprises CD71 expressed on the surface of the cell. In certain embodiments, the cell comprises a stem cell comprising an induced pluripotent stem cell (iPSC), a hematopoietic stem cell (HSC), or a CD4⁺ stem cell.

In certain embodiments, the cell is isolated from a subject suffering from an autoimmune disease. The cell may then be genetically engineered and then delivered to the subject to treat, at least in part, the autoimmune disease. Additionally or alternatively, the cell may be isolated from a healthy subject, genetically engineered, and then delivered to a subject to suffering from an autoimmune disease to treat, at least in part, the autoimmune disease.

In certain embodiments, the cell may be further genetically engineered to reduce its immunogenicity in an allogeneic host. The immune system recognizes specific antigen patterns on the cell surface, e.g., in humans, human leukocyte antigen (HLA) proteins. These patterns of protein antigens are genetically determined and vary between individuals, where an individual's immune system recognizes its own specific antigen pattern as “self” and those antigen patterns that differ as “non-self” or “foreign”. Typically, foreign cells, e.g., allogeneic cells (cells from a genetically dissimilar individual), and/or those demonstrating HLA patterns different than expected, elicit one or more immune responses in the host. In the context of cell therapy applications, this immune response, termed “Host versus Graft” (HvG), can hinder and/or reduce the efficacy of the one or more therapeutic agents as the body recognizes the therapeutic agent as foreign and targets the therapeutic agent for removal. Further, engineered cells, e.g., modified cells, used in cell therapy can recognize the antigen pattern of host cells as foreign and elicit an immune response. This immune response, as herein termed “Graft versus Host” (GvH), can result in the therapy demonstrating a negative and/or harmful effect on the recipient. Thus, provided herein are cells demonstrating reduced immunogenicity. In certain embodiments, provided herein are cells comprising one or more modifications that result in reduced HvG, GvH, and/or both. In certain embodiments, the cells comprises one or more modifications that alters the expression of one or more HLA class one I or class II genes, e.g., (1) one or more genomic modifications that partially or completely inactivates one or more genes that codes for a subunit of an HLA-1 protein, (2) one or more genomic modifications that partially or completely inactivates one or more genes coding for a subunit of an HLA-2 protein or a transcription factor regulating the expression of one or more subunits of an HLA-2 protein. Exemplary modifications are disclosed in US patent applications 2016/0348073, 2022/0162554A1, 2022/0162555, 2015/0017136, 2018/0141992, 2020/0291424, 2017/0016025, 2019/0010514, 2014/0134195, 2019/0381154, 2021/0161971, 2020/0080107, and 2016/0348073; U.S. Pat. Nos. 5,574,205, 8,409,861, 9,695,442, 10,669,557, 11,186,824, and 11,207,393; Lee et al. (2020) SCIENTIFIC REPORTS; Gornalusse (2017) NATURE BIOTECHNOLOGY; Deuse (2019) NATURE BIOTECHNOLOGY; Morton (2020) MOLECULAR THERAPY; Kagoya (2020) CANCER IMMUNOLOGY RESEARCH; Harding (2019) BIORXIV; and Guo (2021) EUROPEAN JOURNAL OF IMMUNOLOGY.

In certain embodiments, the compositions and methods disclosed herein comprise a population of cells. The population of cells can be a homogenous population, wherein the cell population comprises a single type of cells. Alternatively, the population of cells can be a heterogenous population of cells, such as a population of cells isolated from a patient, for example a population of primary blood cells. In certain embodiments, the population of cells comprises at least one population of immunosuppressive immune cells, such as an immunosuppressive T cell, for example a regulatory T cell. In certain embodiments, the population of cells undergoes one or more steps may be performed in order to increase the abundance, e.g., purify and/or separate, of one or more target cells in the population. Any suitable technique may be used to increase the abundance of the one or more target cells from the population for example Fluorescence-automated cell sorting, affinity chromatography, density centrifugation, and the like. In certain embodiments, the target cell comprises at least 50, 60, 70, 75, 80, 85, 90, 95, 96, 97, 98, 99, 99.5, 99.6, 99.7, 99.8, 99.9, or 100% of all the cells in the cell population. In certain embodiments, the cell population further comprises one additional non-target cell population. In certain embodiments, immunosuppressive immune cells comprise at least 50, 60, 70, 75, 80, 85, 90, 95, 96, 97, 98, 99, 99.5, 99.6, 99.7, 99.8, or 99.9% of the cells of the population and the cell population further comprises at least one additional non-target cell population. The non-target cell population can be any suitable non-target cell, such as an effector T cell.

The compositions and methods disclosed herein are surprisingly and unexpectedly advantageous for engineering populations of cells, particularly heterogeneous populations of cells, as the resultant genetically engineered cells demonstrate increased expression of one or more transgenes in immunosuppressive T cells than non-target cells. This safety mechanism ensures that the transgene is expressed primarily in the immunosuppressive cell and primarily in Treg cells that maintain their Treg phenotypes.

### 3. Regulatory Elements

The term “regulatory element,” as used herein, can refer to a transcriptional and/or translational control sequence, such as a promoter, enhancer, transcription termination signal (e.g., polyadenylation signal), internal ribosomal entry sites (IRES), protein degradation signal, or the like, that provide for and/or regulate transcription of a non-coding sequence (e.g., a targeter nucleic acid or a modulator nucleic acid) or a coding sequence (e.g., a Cas protein) and/or regulate translation of an encoded polypeptide. Such regulatory elements are described, for example, in Goeddel, GENE EXPRESSION TECHNOLOGY: METHODS IN ENZYMOLOGY, 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific.

Any suitable promoter can be used, for example a constitutive promoter. In certain embodiments, the promoter comprises a CMV promoter. In certain embodiments, the promoter comprises a MND promoter. In certain embodiments, the promoter comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (115) promoter (optionally with the CMV enhancer), the SV40 promoter, the dihydrofolate reductase promoter, the β-actin promoter, the phosphoglycerol kinase (PGK) promoter, and the EF1α promoter. A TERT, LGR5, and/or PHGDH gene from any suitable species, e.g., a homolog can be used. In preferred embodiments, the gene is syngeneic to the cell, more preferably the gene is a nonhuman gene.

In certain embodiments, the promoter comprises an endogenous FOXP3 promoter. In this case, the compositions and methods disclosed herein demonstrate surprising and unexpected advantages, particularly that expression of a transgene is increased in FOXP3⁺ immunosuppressive T cells, e.g., Tregs, than non-target cells. When using Tregs as a therapy, this provides a useful safety mechanism that ensure transgene expression occurs primarily in therapeutic cell population rather than any non-therapeutic cell type, e.g., Teff, that may have been genetically engineered during the manufacturing process. Additionally or alternatively, it ensures that transgene expression occurs primarily in Treg cells that maintain their Treg phenotypes as Tregs have been demonstrated to differentiate and/or convert into other immune cell types.

### 4. Foxp3 Functional Derivatives

In certain embodiments, the transgene encodes a derivative of a functional derivative of a foxp3 polypeptide. The functional derivative can be any suitable derivative of foxp3, for example a foxp3 that comprises one or more modifications whereby the stability of the foxp3 polypeptide demonstrates increased stability, for example at least 10, 20, 30, 40, 50, 60, 70, 80, 90, 100, 200, or 500% and/or not more than 2000% stable as compared to wildtype foxp3. Any suitable modification can be used to increase the stability of the foxp3 protein, such as a mutation to a ubiquitination or deubiquitination site, for example amino acids 19, 33, 41, 175, 227, 250, 252, 263, 268, 418, and/or 422.

### 5. Transgenes

Any suitable transgene can be inserted into the genome of the cell, for example a cytokine, an interleukin, a cytokine receptor, a transcription factor, a chimeric receptor, a reporter protein, a selectable marker, or a combination thereof. In certain embodiments, the transgene comprises a Treg healing factor. The transgene can encode any suitable number of polypeptides, for example at least 1, 2, 3, 4, 5, 6, 7, 8, or 9 and/or not more than 20, 15, 10, 9, 8, 7, 6, 5, 4, 3, or 2 polypeptides.

Any suitable database can be used to derive a suitable transgene sequence, for example GenBank, PubMed, and/or KEGG GENES Database, and the like. A skilled artisan would understand how to identify and use such databases to identify and retrieve sequences of interest from the databases. Additionally or alternatively genes of interest can be identified using any suitable technique, such as sequencing a sample from a cell of interest or subject of interest. Suitable techniques include, but are not limited to, Sanger sequencing and high throughput sequencing.

a. Chimeric Receptors

In certain embodiments, the transgene encodes a chimeric antigen receptor (CAR) or component thereof. In certain embodiments, the transgene encodes a T-cell receptor (TCR).

Any suitable CAR can be used depending on the application, such as a CAR that binds one or more targets on one or more target cells involved in autoimmune disease. Further, the CAR can comprise any suitable binding domain, such as an antibody, a nanobody (VHH), a Fab, a scFv, diabody, a triabody, a minibody, a single-domain antibody. In certain embodiments, the CAR comprises a first-generation CAR, a second-generation CAR, a third-generation CAR, and/or a fourth-generation CAR. Any suitable antigen binding domain can be combined with any suitable combination of additional CAR domains as necessary for the application, for example a tandem CAR. In certain embodiments, the cell comprises a cell that is autologous to a potential recipient of the cell. In certain embodiments, the cell comprises a cell demonstrating reduced immunogenicity when placed in an allogeneic host, preferably the cell is non-immunogenic when placed in an allogeneic host.

In certain embodiments, the CAR can be an antibody, an antigen-binding fragment of an antibody, or a fusion protein derived from such an antibody, such as a single-chain variable fragment (scFv). In accordance with these embodiments herein, a scFv can include a single chain Fv antibody in which the variable domains of the heavy chain and of the light chain of a traditional two chain antibody have been joined to form a single polypeptide chain. In some embodiments, single chain antibodies contemplated herein can be derived from any species including human or animal (e.g., mice, rabbit, pig, dog, cow, horse, goat, camel, or other animal). In some embodiments, the intracellular signaling domain can include a signaling domain and a co-stimulatory domain.

In certain embodiments, the CAR further comprises a spacer domain (hinge domain) which links an antigen binding domain to a transmembrane domain. In some embodiments, a spacer domain of appropriate length can improve mobility of an antigen binding domain to allow for optimal binding to a target antigen and improve flexibility. In certain embodiments, the spacer domain comprises at least a portion or segment of a hinge region of an IgG1, IgG2, IgG3, or IgG4. In some embodiments, a spacer domain can be derived from a CH2 region and/or CH3 region of an IgG1, IgG2, IgG3, or IgG4. In certain embodiments, the spacer domain comprises upper hinge amino acids found between the variable heavy chain and the core and the core hinge amino acids including a polyproline region. In certain embodiments, the spacer region comprises at least a portion of a hinge region of a human IgG4 binge spacer. In some embodiments, the spacer region comprises a human IgG4 hinge-CH3 spacer.

In some embodiments, the CAR further comprises a transmembrane domain. A transmembrane domain can provide anchoring of a CAR in a cell membrane and further assist in translocation of a signal from an extracellular domain to an intracellular domain. In some embodiments, the transmembrane domain comprises a membrane-bound or transmembrane protein. In certain embodiments, the transmembrane domain comprises a transmembrane region of an alpha, beta, or zeta chain of a T-cell receptor, such as CD28, CD3, CD45, CD4, CD 8, CD9, CD 16, CD22, CD33, CD37, CD64, CD80, CD86, CD134, CD137 or CD154. In some embodiments, the transmembrane domain comprises a CD28 transmembrane domain (CD28tm).

In certain embodiments, the CAR further comprises an intracellular signaling domain linked to a transmembrane domain. In accordance with these embodiments, the intracellular signaling domain can activate a function of a cell when the antigen binding domain binds to a target antigen. In some embodiments, the intracellular signaling domain can activate a function of a cell expressing a CAR, such as a T cell expressing the CAR. In certain embodiments, the intracellular signaling domain comprises one or more intracellular signaling domains. In some embodiments, the intracellular signaling domain comprises a functional domain of a primary cytoplasmic signaling protein. In some embodiments, the intracellular signaling domain comprises a functional domain of a primary cytoplasmic signaling protein, and at least one functional domain of one or more secondary cytoplasmic signaling proteins. In certain embodiments, a primary cytoplasmic signaling protein that acts in a stimulatory manner can contain signaling motifs which are known as intracellular receptor tyrosine-based activation motifs (ITAMs). In accordance with these embodiments, examples of ITAMs containing primary cytoplasmic signaling domains for use herein include, but are not limited to, those derived from CD3zeta, FcR gamma, CD3 gamma, CD3 delta, CD3 epsilon, CD5, CD22, CD79a, CD79b, or CD66d. In some embodiments, the intracellular signaling domain and/or the co-stimulatory domain herein can include all or a biologically active fragment of CD27, CD28, 41BB, 0X40, CD30, CD40, ICOS, lymphocyte function-associated antigen-1 (LFA-1), CD2, CD7, LIGHT, NKG2C, or B7H3, and/or a ligand that specifically binds with CD83. In some embodiments, the intracellular signaling domain herein can include all or a biologically relevant segment of the signaling domain of CD3-zeta or variant thereof and all or a portion of the signaling domain of 4-1BB or variant thereof.

In certain embodiments, the first cell further comprises a second CAR, or a polynucleotide coding for at least a portion thereof, that binds a second binding partner or portion thereof different from the first, such as a bicistronic CAR cell. The first cell can comprise any suitable number of additional CARs that bind to binding partners or portions thereof different from the others. In certain embodiments, the first cell comprises (CAR)ₓ, or a polynucleotide coding for at least a portion thereof, wherein, for each integer x. (CAR)ₓ comprises a CAR that bind to binding partners or portions thereof different from the other CARS. In certain embodiments, the integer x is at least 1, 2, 3, 4, 5, 6, 7, 8, 9, 10, 11, 12, 13, or 14 and/or not more than 2, 3, 4, 5, 6, 7, 8, 9, 10, 11, 12, 13, 14, or 15, for example 1-15, preferably 1-10, more preferably 1-5, even more preferably 1-3. For example, for an integer of 3, the first cell comprises 3 CARs, each of which bind to binding partners or portions thereof different from the others.

The CAR can comprise any suitable affinity for a binding partner or portion thereof. In certain embodiments, the CAR binds to the binding partner or portion thereof with an affinity of at least 0.001, 0.005, 0.01, 0.05, 0.1, 0.2, 0.3, 0.4, 0.5, 0.6, 0.7, 0.8, 0.9, 1, 5, 10, 50, 100, or 1,000 pM and/or no more than 0.005, 0.01, 0.05, 0.1, 0.2, 0.3, 0.4, 0.5, 0.6, 0.7, 0.8, 0.9, 1, 5, 10, 50, 100, 1,000, 10,000 pM, for example 0.001 to 10,000 pM, preferably 100-10,000 pM, more preferably 0.001 to 1 pM, even more preferably 1 to 100 pM. Not wishing to be bound by theory, the affinity of a CAR may be tailored for an intended application such that CAR leads to activation at the site of use more often than at an unintended site, for example too high of an affinity may allow for a CAR to be activated at a tissue expressing a low amount of the binding partner as well as at the site of use where a higher amount of the binding partner is present.

b. Chemokine Receptors

In certain embodiments, the transgene encodes a chemokine receptor, such as a cytokine receptor, for example CCR and/or a CXCR receptor. Any suitable chemokine receptor can be used, for example CXC chemokine receptor 1 (CXCR1), CXC chemokine receptor 2 (CXCR2), CXC chemokine receptor 3 (CXCR3), CXC chemokine receptor 4 (CXCR4), CXC chemokine receptor 5 (CXCR5), CXC chemokine receptor 6 (CXCR6), CC chemokine receptor 1 (CCR1), CC chemokine receptor 2 (CCR2), CC chemokine receptor 3 (CCR3), CC chemokine receptor 4 (CCR4), CC chemokine receptor 5 (CCR5), CC chemokine receptor 6 (CCR6), CC chemokine receptor 7 (CCR7), CC chemokine receptor 8 (CCR8), CC chemokine receptor 9 (CCR9), CC chemokine receptor 10 (CCR10), CC chemokine receptor 11 (CCR11), C chemokine receptor (XCR1), CX₃C chemokine receptor (CX3CR1), or a combination thereof or biologically active fragment thereof. In preferred embodiments, the chemokine receptor comprises a receptor involved in an autoimmune disease. Preferably, the chemokine receptor comprises CCR7 and/or CCR9.

### C. Cytokines and Cytokine Receptors

In certain embodiments, the transgene encodes a cytokine, for example IL-10, TGF-beta, and/or PD-10. The cytokine can be any suitable cytokine as needed from the application, such as an immune checkpoint inhibitor, for example PDCD1 (PD-1), CTLA4, ADORA2A (A2AR), B7-H3, B7-H4, BTLA, KIR, LAG3, HAVCR2 (TIM3), TIGIT, VISTA, PTPN6 (SHP-1), and FAS. In certain embodiments, the transgene encodes one or more cytokine receptors, for example CD25. In certain embodiments, the transgene encodes both a cytokine and a cytokine receptor, for example IL-2 and CD25. In certain embodiments, the transgene encodes, IL-13, IL-22, AREG, and/or BDNF.

### 6. Untranslated Regions

Any suitable UTR may be used. In certain embodiments, the nucleotide sequence comprises a heterologous 3′ UTR. Any suitable heterologous 3′ UTR can be used, for example a SV40 reversed, bGH, WPRE, WPRE3/SV40p128, SV40 corrected, WPRE3/SV40.2, TCRA EIF3, or s27a3RU, preferably a SV40 reversed, SV40 corrected, or bGH 3′ UTR. 3′ UTR modifications are described, for example, in Zufferey et al. (1990) J. VIROLOGY; Choi et al. (2014) MOLECULAR BRAIN; DeSilva et al. (2021) ELIFE; Zeng et al. (2020) ADV. MAT.

TABLE 1
exemplary 3′ UTR sequences

| | SEQ | |
|---|---|---|
| | ID | |
| Name | NO | Sequence |
| SV40 | 15 | agatgataagatacattgatgagtttggacaaacca |
| for | | caactagaatgcagtgaaaaaaatgctttatttgtg |
| | | aaatttgtgatgctattgctttatttgtaaccatta |
| | | taagctgcaataaacaagtt |
| SV40 | 16 | aacttgtttattgcagcttataatggttacaaataa |
| rev | | agcaatagcatcacaaatttcacaaataaagcattt |
| | | ttttcactgcattctagttgtggtttgtccaaactc |
| | | atcaatgtatcttatcatct |
| W3p128 | 17 | gataatcaacctctggattacaaaatttgtgaaaga |
| | | ttgactggtattcttaactatgttgctccttttacg |
| | | ctatgtggatacgctgctttaatgcctttgtatcat |
| | | gctattgcttcccgtatggctttcattttctcctcc |
| | | ttgtataaatcctggttagttcttgccacggcggaa |
| | | ctcatcgccgcctgccttgcccgctgctggacaggg |
| | | gctcggctgttgggcactgacaattccgtggtgtta |
| | | gatgataagatacattgatgagtttggacaaaccac |
| | | aactagaatgcagtgaaaaaaatgctttatttgtga |
| | | aatttgtgatgctattgctttatttgtaaccattat |
| | | aagctgcaataaacaagtt |

In certain embodiments, the 5′ UTR is modified to (1) increase of stability of the transcript via addition of transcription factor binding sites, alternative capping sites or cap-independent motifs (such as IRESs), inclusion of tertiary structures and intermolecular RNA complexing elements, and elements to promote or repress circular-RNA formation, (ii) include synthetically derived promoters, (iii) increase translation initiation by engineering of a regulatory element, such as a Kozak sequence, and/or (iv) increase of translation efficiency of the transcript by inclusion of additional binding sites for transcriptional machinery (such as EIF binding sites including but not limited to EIF3 and EIF4a).

### D. Compositions and Methods for Engineering Cells

Provided herein are compositions comprising (1) a nucleic acid-guided nuclease system comprising: (a) a nucleic acid-guided nuclease, or a polynucleotide encoding the nuclease; and (b) a guide nucleic acid (gNA), or a polynucleotide encoding the gNA, comprising a spacer sequence complementary to a site within or near the FOXP3 gene; and (2) a donor template encoding a transgene. In certain embodiments, the site is upstream of the FOXP3 gene, preferably at least 1, 2, 3, 6, 9, 12, 15, 30, 60, 61, 90, 120, 240, or 480 bases and/or not more than 1920, 960, 480, 240, 120, or 90 bases upstream of the FOXP3 gene, more preferably about 61 to 1 bases before the start codon of the FOXP3 gene. In certain embodiments, the site is inserted immediately upstream of the start codon of the FOXP3 gene. In certain embodiments, the site is inserted downstream of the FOXP3 gene, preferably at least 1, 2, 3, 6, 9, 12, 15, 30, 60, 61, 90, 120, 240, or 480 bases and/or not more than 1920, 960, 480, 240, 120, or 90 bases downstream of the FOXP3 gene, more preferably about 61 to 1 bases after the stop codon of the FOXP3 gene. In certain embodiments, the site is inserted immediately after the stop codon of the FOXP3 gene. The transgene can be any suitable transgene, such as a transgene as disclosed herein, for example a cytokine, interleukin, cytokine receptor, transcription factor, and/or chimeric receptor, preferably a CAR or a CAR component, a cytokine, a self-cleaving peptide, or a combination thereof as disclosed herein. The spacer sequence of the gNA can be any suitable sequence, such as any one of SEQ ID NOs: 1-14. The nuclease can be any suitable nuclease, such as a Class 1 or Class 2 nuclease, for example a Type II, e.g., cas9, or a Type V nuclease, e.g., a Type V-A, for example cpf1, V-B, V-C, V-D, or V-E nuclease. In certain embodiments, the nuclease further comprises one or more functional domains, such as a nuclear localization signal.

TABLE 2
exemplary spacer sequences

| Name | SEQ ID NO | Sequence |
|---|---|---|
| FOXP3-58 | 1 | AAGGAGTCACACGTGTGCTA |
| FOXP3 Stop_2 | 2 | ACCTCAAGATCAAGGAAAGG |
| FOXP3 Start_2 | 3 | ACGCAGCCTGCCCTTGGACA |
| FOXP3 Stop_3 | 4 | CAAGATCAAGGAAAGGAGGA |
| FOXP3 ATG_1 | 5 | CAGGCCTGGGGTTGGGCATC |
| FOXP3 ATG_2 | 6 | CCAGGCCTGGGGTTGGGCAT |
| FOXP3 Stop_1 | 7 | CTGACCTCAAGATCAAGGAA |
| FOXP3-39 | 8 | CTGCGTAGACAATAGGGGAA |
| FOXP3-61 | 9 | GAGTCACACGTGTGCTAGGG |
| FOXP3-33 | 10 | GGCAGGCTGCGTAGACAATA |
| FOXP3 Start_1 | 11 | GGCATCGGGTCCTTGTCCAA |
| FOXP3-16 | 12 | TCGGGTCCTTGTCCAAGGGC |
| FOXP3 Start | 13 | TTGGACAAGGACCCGATGCC |
| FOXP3-17 | 14 | TTGTCTACGCAGCCTGCCCT |

Provided herein is a method for editing a genome, e.g., genetically engineering and/or modifying, of a cell (or population of cells) comprising contacting the cell with a composition as disclosed herein, whereby the resulting cell comprises a heterologous polynucleotide in a FOXP3 locus. The cell can be any suitable cell, such as a cell or a derivative or progeny of a cell as disclosed herein, such as a eukaryotic cell, preferably a metazoan cell, more preferably a human cell. In certain embodiments, the cell comprises an immune cell, such as a neutrophil, eosinophil, basophil, mast cell, monocyte, macrophage, dendritic cell, natural killer cell, T cell, or a lymphocyte, or an immune precursor cell, such as a stem cell, an induced pluripotent stem cell, a hematopoietic stem cell, or a CD4⁺ stem cell. In certain embodiments, the immune cell comprises a T cell, preferably an immunosuppressive T cell, more preferably a Treg. In certain embodiments, the cell is isolated from an individual, such as an individual suffering from a disease or a health individual. The terms “individual” and “subject” may be used interchangeably. In certain embodiments, the disease comprises a disease as disclosed herein, preferably an autoimmune disease, such as intestinal inflammation, for example IBD, CD, or UC. In certain embodiments, the cell is further engineered to not promoter an immune response in an allogeneic individual as disclosed herein. In certain embodiments, the method results in one or more phenotypic enhancements of the cell. In certain embodiments, after editing, expression of foxp3 and/or the transgene in the cell is at least 1.1, 1.2, 1.3, 1.4, 1.5, 1.6, 1.7, 1.8, 1.9, 2, 2.2, 2.4, 2.6, 2.8, 3, 3.5, 4, or 4.5-fold and/or not more than 1.2, 1.3, 1.4, 1.5, 1.6, 1.7, 1.8, 1.9, 2, 2.2, 2.4, 2.6, 2.8, 3, 3.5, 4, 4.5, or 5-fold greater than a cell comprising the wildtype FOXP3 3′ UTR, for example 1.1 to 5-fold, preferably 1.1 to 3-fold, more preferably 1.5 to 2-fold. In certain embodiments, after editing, the cell demonstrates no more than 50, 40, 30, 20, 10, 9, 8, 7, 6, 5, 4, 3, 2, 1 or, 0% expression of a transgene within FOXP3 when measured as an effector T-cell as compared to when measured as an immunosuppressive T-cell.

### 1. Nucleic Acid-Guided Nuclease Systems

The present invention provides compositions and methods for genetically modifying a genome of a cell using a nucleic acid-guided nuclease system, e.g., CRISPR-cas, preferably at a preselected target locus. In certain embodiments, the method comprises delivering the nucleic acid-guide nuclease system, or one or more polynucleotides encoding one or more components of the system, to a cell, wherein the nuclease contacts the preselected target locus thereby effecting a change. The change can be any suitable change, for example introduction of one or more strand breaks and subsequent introduction of one or more genetic modifications or effecting a change in expression of nearby genes, e.g., trans-activation or silencing. Cleavage of a target nucleotide sequence in the genome of a cell by a CRISPR-Cas system or complex can activate DNA damage pathways, which may rejoin the cleaved DNA fragments by non-homologous end joining (NHEJ) or homology directed repair (HDR). HDR requires a repair template, e.g., a recombinant nucleic acid, either endogenous or exogenous, to transfer the sequence information from the repair template to the target. In certain embodiments, a strand break is repaired by NHEJ, an inherently error-prone process, that results in one or more mutations at or near the strand break. In certain embodiments, a strand break is repaired by HDRn using at least a portion of a recombinant nucleic acid. In this case, the recombinant nucleic acid can comprise any suitable sequence.

Any suitable nucleic acid-guided nuclease system, e.g., CRISPR-Cas system, can be used, as used herein a nucleic acid-guided nuclease. In certain embodiments, the cell comprises a nucleic acid-guided nucleases, or a polynucleotide encoding the nuclease, and a heterologous guide nucleic acid (gNA), or a polynucleotide encoding the gNA. Typically, the guide nucleic acid comprises a spacer sequence, wherein the spacer sequence is complementary to a site to which it is intended to bind. In certain embodiments, the spacer sequence is complementary to a site upstream and/or downstream of a FOXP3 gene and/or a heterologous locus, for example a safe harbor site. The gNA should be compatible with the nuclease, i.e., capable of binding to the nuclease and forming a nucleic acid-guide nuclease complex. In certain embodiments, the nuclease is functional, whereby upon binding to the site is capable of effecting one or more strand breaks in the polynucleotide at or near the site. Alternatively, the nucleic acid-guided nuclease is catalytically inactive such that it binds at the site but is incapable of effecting one or more strand breaks. In certain embodiments, the nucleic acid-guided nuclease comprises a heterologous domain. The heterologous domain can be any suitable domain for the intended function, for example a CRISPR base editor comprising a deaminase domain, a CRIPSR silencer, a CRISPR trans activator, and the like. The nucleic acid-guided nucleic can be any suitable nuclease, for example a Class I or a Class II nuclease, such as a Type II, e.g., cas9 or a Type V nuclease, such as a Type Va, Vb, or Vc nuclease, e.g., cas12. In certain embodiments, the nuclease comprises a CRISPR-cas transactivator that binds to a site upstream of a FOXP3 gene, increasing and/or stabilizing the expression of the FOXP3 gene, for example nucleic acid-guided nuclease fused to a TET demethylase enzyme.

a. Nucleases

The terms “CRISPR-Associated protein,” “Cas protein,” and “Cas,” as used interchangeably herein, can refer to a naturally occurring Cas protein or an engineered Cas protein. Non-limiting examples of Cas protein engineering include but are not limited to mutations and modifications of the Cas protein that alter the activity of the Cas, alter the PAM specificity, broaden the range of recognized PAMs, and/or reduce the ability to modify one or more off-target loci as compared to a corresponding unmodified Cas. In certain embodiments, the altered activity of engineered Cas comprises altered ability (e.g., specificity or kinetics) to bind a naturally occurring gNA, e.g., gRNA or engineered gNA, e.g., gRNA, altered ability (e.g., specificity or kinetics) to bind a target nucleotide sequence, altered processivity of nucleic acid scanning, and/or altered effector (e.g., nuclease) activity. A Cas protein having nuclease activity can be referred to as a “CRISPR-Associated nuclease” or “Cas nuclease”, “nucleic acid-guided nuclease”, or simply “nuclease,” as used interchangeably herein.

In certain embodiments, a Cas protein comprises one or more effector domains. The one or more effector domains may be located at or near the N-terminus of the Cas protein and/or at or near the C-terminus of the Cas protein. In certain embodiments, an effector domain comprised in the Cas protein is a transcriptional activation domain (e.g., VP64), a transcriptional repression domain (e.g., a KRAB domain or an SID domain), an exogenous nuclease domain (e.g., FokI), a deaminase domain (e.g., cytidine deaminase or adenine deaminase), or a reverse transcriptase domain (e.g., a high fidelity reverse transcriptase domain). Other activities of effector domains include but are not limited to methylase activity, demethylase activity, transcription release factor activity, translational initiation activity, translational activation activity, translational repression activity, histone modification (e.g., acetylation or demethylation) activity, single-stranded RNA cleavage activity, double-strand RNA cleavage activity, single-strand DNA cleavage activity, double-strand DNA cleavage activity, and nucleic acid binding activity.

In certain embodiments, a Cas protein comprises one or more protein domains that enhance homology-directed repair (HDR) and/or inhibit non-homologous end joining (NHEJ). Exemplary protein domains having such functions are described in Jayavaradhan et al. 2019 Nature Communications and Janssen et al. 2019 Molecular Therapy—Nucleic Acids. In certain embodiments, a Cas protein comprises a dominant negative version of p53-binding protein 1 (53BP1), for example, a fragment of 53BP1 comprising a minimum focus forming region (e.g., amino acids 1231-1644 of human 53BP1). In certain embodiments, a Cas protein comprises a motif that is targeted by APC-Cdh1, such as amino acids 1-110 of human Geminin, thereby resulting in degradation of the fusion protein during the HDR non-permissive G1 phase of the cell cycle.

In certain embodiments, a Cas protein comprises an inducible or controllable domain. Non-limiting examples of inducers or controllers include light, hormones, and small molecule drugs. In certain embodiments, a Cas protein comprises a light inducible or controllable domain. In certain embodiments, a Cas protein comprises a chemically inducible or controllable domain.

b. Guide Nucleic Acids

A guide nucleic acid can be a single gNA (sgNA, e.g., chimeric guide RNA (chiRNA)), in which the gNA is a single polynucleotide, or comprise more than one separate polynucleotides that when combined are capable of activating a Cas nuclease. In certain embodiments, a single guide nucleic acid is capable of activating a Cas nuclease alone (e.g., in the absence of a tracrRNA).

Guide nucleic acid sequences that are operative with a type II or type V Cas protein are known in the art and are disclosed, for example, in U.S. Pat. Nos. 9,790,490, 9,896,696, 10,113,179, and 10,266,850, and U.S. Patent Application Publication No. 2014/0242664. It is understood that these sequences are merely illustrative, and other guide nucleic acid sequences may also be used with these Cas proteins.

In certain embodiments some or all of the gNA is RNA, e.g., a gRNA. In certain embodiments, 5-100%, 10-100%, 20-100%, 30-100%, 40-100%, 50-100%, 60-100%, 70-100%, 80-100%, 90-100%, 95-100%, 99-100%, 99.5-100% of the gNA is gRNA. In certain embodiments, 20%-80%, 20%-70%, 20%-60%, 20%-50%, 20%-40%, 20%-30%, 30%-80%, 30%-70%, 30%-60%, 30%-50%, 30%-40%, 40%-80%, 40%-70%, 40%-60%, 40%-50%, 50%-80%, 50%-70%, 50%-60%, 60%-80%, 60%-70%, or 70%-80% of gNA is RNA. In certain embodiments, 50% of the gNA is RNA. In certain embodiments, 70% of the gNA is RNA. In certain embodiments, 90% of the gNA is RNA. In certain embodiments, 100% of the gNA is RNA, e.g., a gRNA. In further embodiments, the remaining portion of the gNA that is not RNA comprises a modified ribonucleotide, a deoxyribonucleotide, a modified deoxyribonucleotide, or a synthetic, e.g., unnatural nucleotide, for example, not intended to be limiting, threose nucleic acid, locked nucleic acid, peptide nucleic acid, arabinonucleic acid, hexose nucleic acid, among others. Exemplary modifications are disclosed in U.S. Pat. Nos. 10,900,034 and 10,767,175, U.S. Patent Application Publication No. 2018/0119140, Watts et al. 2008 Drug Discovery Today, and Hendel et al. 2015) Nature Biotechnology 33:985. Additional modifications are disclosed in Dang et al. 2015 Genome Biology, Kocaz et al. 2019 Nature Biotechnology, Liu et al. 2019 Nucleic Acids Research, Schubert et al. 2018 Journal of Cytokine Biology, Teng et al. 2019 Genome Biology, and Wu et al. 2018 Cellular and Molecular Life Sciences.

The gNA can further comprise a protective nucleotide sequence that prevents or reduces nucleic acid degradation. In certain embodiments, the protective nucleotide sequence is at least 5 (e.g., at least 10, at least 15, at least 20, at least 25, at least 30, at least 35, at least 40, at least 45, or at least 50) nucleotides in length. The length of the protective nucleotide sequence increases the time for an exonuclease to reach the 5′ tail, modulator stem sequence, targeter stem sequence, and/or spacer sequence, thereby protecting these portions of the single guide nucleic acid, the modulator nucleic acid, and/or the targeter nucleic acid from degradation by an exonuclease. In certain embodiments, the protective nucleotide sequence forms a secondary structure, such as a hairpin or a tRNA structure, to reduce the speed of degradation by an exonuclease (see, for example, Wu et al. 2018 Cellular and Molecular Life Sciences). Secondary structures can be predicted by methods known in the art, such as the online webserver RNAfold developed at University of Vienna using the centroid structure prediction algorithm (see, Gruber et al. 2008 Nucleic Acids Research).

c. Repair Templates

Cleavage of a target nucleotide sequence in the genome of a cell by a CRISPR-Cas system or complex can activate DNA damage pathways, which may rejoin the cleaved DNA fragments by non-homologous end joining (NHEJ) or homology directed repair (HDR). HDR requires a repair template, e.g., a recombinant nucleic acid, either endogenous or exogenous, to transfer the sequence information from the repair template to the target. In certain embodiments, the repair template comprises a recombinant nucleic acid.

In certain embodiments, the repair template is complementary to a polynucleotide comprising the target nucleotide sequence or a portion thereof. When optimally aligned, a repair template may overlap with one or more nucleotides of a target nucleotide sequences (e.g., about or more than about any of 1, 5, 10, 15, 20, 25, 30, 35, 40, or more nucleotides). The nucleotide sequence of the repair template is typically not identical to the genomic sequence that it replaces. Rather, the repair template may contain one or more substitutions, insertions, deletions, inversions or rearrangements with respect to the genomic sequence, so long as sufficient homology is present to support homology-directed repair. In certain embodiments, the repair template comprises a non-homologous sequence flanked by two regions of homology (i.e., homology arms), such that homology-directed repair between the target DNA region and the two flanking sequences results in insertion of the non-homologous sequence at the target region. In certain embodiments, the repair template comprises a non-homologous sequence 10-100 nucleotides, 50-500 nucleotides, 100-1,000 nucleotides, 200-2,000 nucleotides, or 500-5,000 nucleotides in length positioned between two homology arms.

Generally, the homologous region(s) of a repair template has at least 50% sequence identity to a genomic sequence with which recombination is desired. The homology arms are designed or selected such that they are capable of recombining with the nucleotide sequences flanking the target nucleotide sequence under intracellular conditions. In certain embodiments, where HDR of the non-target strand is desired, the repair template comprises a first homology arm homologous to a sequence S′ to the target nucleotide sequence and a second homology arm homologous to a sequence 3′ to the target nucleotide sequence. In certain embodiments, the first homology arm is at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to a sequence S′ to the target nucleotide sequence. In certain embodiments, the second homology arm is at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to a sequence 3′ to the target nucleotide sequence. In certain embodiments, when the repair template sequence and a polynucleotide comprising a target nucleotide sequence are optimally aligned, the nearest nucleotide of the repair template is within about any of 1, 5, 10, 15, 20, 25, 50, 75, 100, 200, 300, 400, 500, 1000, 2000, 3000, 4000, or more nucleotides from the target nucleotide sequence.

In certain embodiments, the repair template further comprises an engineered sequence not homologous to the sequence to be repaired. Such engineered sequence can harbor a barcode and/or a sequence capable of hybridizing with a repair template-recruiting sequence disclosed herein.

In certain embodiments, the repair template further comprises one or more mutations relative to the genomic sequence, wherein the one or more mutations reduce or prevent cleavage, by the same CRISPR-Cas system, of the repair template or of a modified genomic sequence with at least a portion of the repair template sequence incorporated. In certain embodiments, in the repair template, the PAM adjacent to the target nucleotide sequence and recognized by the Cas nuclease is mutated to a sequence not recognized by the same Cas nuclease. In certain embodiments, in the repair template, the target nucleotide sequence (e.g., the seed region) is mutated. In certain embodiments, the one or more mutations are silent with respect to the reading frame of a protein-coding sequence encompassing the mutated sites.

The repair template can be provided to the cell as single-stranded DNA, single-stranded RNA, double-stranded DNA, or double-stranded RNA. It is understood that a CRISPR-Cas system, such as a system disclosed herein, may possess nuclease activity to cleave the target strand, the non-target strand, or both. When HDR of the target strand is desired, a repair template having a nucleic acid sequence complementary to the target strand is also contemplated. The repair template can be introduced into a cell in linear or circular form. If introduced in linear form, the ends of the repair template may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends (see, for example, Chang et al. 1987 Proceedings of the National Academy of Sciences; Nehls et al. 1996 Science; see also the chemical modifications for increasing stability and/or specificity of RNA disclosed supra). Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. For example, linearly covalently closed DNA, for example as generated by a telomerase enzyme like TelN, can be used as a repair template. As an alternative to protecting the termini of a linear repair template, additional lengths of sequence may be included outside of the regions of homology that can be degraded without impacting recombination.

A repair template can be a component of a vector as described herein, contained in a separate vector, or provided as a separate polynucleotide, such as an oligonucleotide, linear polynucleotide, or synthetic polynucleotide. In certain embodiments, the repair template is a DNA. In certain embodiments, a repair template is in the same nucleic acid as a sequence encoding the single guide nucleic acid, a sequence encoding the targeter nucleic acid, a sequence encoding the modulator nucleic acid, and/or a sequence encoding the Cas protein, where applicable. In certain embodiments, a repair template is provided in a separate nucleic acid. A repair template polynucleotide may be of any suitable length, such as about or at least about any of 50, 75, 100, 150, 200, 500, 1000, 2000, 3000, 4000, or more nucleotides in length.

d. Delivery

Any suitable method can be used to deliver polynucleotides and/or proteins to a cell. Methods of introducing polynucleotides and/or proteins into a cell include physical, biological, and chemical methods. Physical methods for introducing polynucleotides and/or proteins into a host cell include calcium phosphate precipitation, lipofection, particle bombardment, microinjection, electroporation, viral transduction, and the like, Polynucleotides and/or proteins can be introduced into target cells using commercially available methods which include electroporation (Amaxa Nucleofector-II (Amaxa Biosystems, Cologne, Germany), BTX ECM 830 (Harvard Instruments, Boston, MA, USA), Gene Pulser II (BioRad, Denver, CO, USA), Multiporator (Eppendorf, Hamburg, Germany), among others). Polynucleotides and/or proteins can also be introduced into cells using cationic liposome mediated transfection using lipofection, using polymer encapsulation, using peptide mediated transfection, or using biolistic particle delivery systems such as “gene guns” (Nishikawa (2001) HUM GENE THER).

Biological methods for introducing polynucleotides and/or proteins of interest into a host cell include the use of vectors. Viral vectors can be derived from retrovirus, lentivirus, poxviruses, herpes simplex virus I, adenoviruses, and adeno-associated viruses, and the like. Sec, for example, U.S. Pat. Nos. 5,350,674 and 5,585,362.

Chemical means for introducing polynucleotides and/or proteins into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma (St. Louis, MO, USA); dicetyl phosphate (“DCP”) can be obtained from K & K Laboratories (Plainview, NY USA); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL, USA). Stock solutions of lipids in chloroform or chloroform/methanol can be stored at about −20° C. Chloroform can be as a solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh (1991) GLYCOBIOLOGY). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Once delivered to the cell, any suitable method can be used for integrating at least a portion of the transgene the genome of the cell, such as viral and/or CRISPR/Cas-mediated.

In certain embodiments, the nucleic acid-guided nuclease and the gNA form a nucleic acid-guided nuclease complex. The complex may be delivered to a cell by introducing a pre-formed ribonucleoprotein (RNP) complex into the cell. Alternatively, one or more components, e.g., a polynucleotide encoding the component, of the complex may be expressed in the cell. Exemplary methods of delivery are known in the art and described in, for example, U.S. Pat. Nos. 8,697,359, 10,113,167, 10,570,418, 10,829,787, 11,118,194, and 11,125,739 and U.S. Patent Application Publication Nos. 2015/0344912, 2018/0119140, and 2018/0282763.

The nuclease system and/or repair template can be delivered to a cell by any suitable delivery method, such as a viral or non-viral method disclosed herein. In certain embodiments, nuclease system and/or repair template is introduced into the target cell as one or more naked polynucleotides or in complex with a liposome or poloxamer. In certain embodiments, a nuclease system and/or repair template is introduced into the target cell by electroporation. In other embodiments, a nuclease system and/or repair template is introduced into the target cell by infection, e.g., transduction. The engineered, non-naturally occurring system can be delivered before, after, or simultaneously with the repair template. A skilled person in the art will be able to choose proper timing based upon the form of delivery (consider, for example, the time needed for transcription and translation of RNA and protein components) and the half-life of the molecule(s) in the cell.

### 2. Viral Systems

In certain embodiments, the recombinant nucleic acids are comprised in a viral vector, preferably a replication incompetent viral vector. Any suitable viral vector can be used. In preferred embodiments, the viral vector comprises a lentiviral vector.

Viral vector delivery systems include DNA and RNA viruses, which have either episomal or integrated genomes after delivery to the cell. Gene therapy procedures are known in the art and disclosed in Van Brunt 1988 Biotechnology; Anderson 1992 Science; Nabel & Feigner 1993 TIBTECH; Mitani & Caskey 1993 TIBTECH; Dillon 1993 TIBTECH; Miller 1992 Nature; Vigne 1995 Restorative Neurology and Neuroscience 35; Kremer & Perricaudet 1995 British Medical Bulletin; Haddada et al. 1995 Current Topics in Microbiology and Immunology; Yu et al. 1994 Gene Therapy; and Doerfler and Bohm (Eds.) 2012 The Molecular Repertoire of Adenoviruses II: Molecular Biology of Virus-Cell Interactions. In certain embodiments, at least one of the vectors is a DNA plasmid. In certain embodiments, at least one of the vectors is a viral vector (e.g., retrovirus, adenovirus, or adeno-associated virus).

Certain vectors are capable of autonomous replication in a host cell into which they are introduced (e.g., bacterial vectors having a bacterial origin of replication and episomal mammalian vectors). Other vectors (e.g., non-episomal mammalian vectors and replication defective viral vectors) do not autonomously replicate in the host cell. Certain vectors, however, may be integrated into the genome of the host cell and thereby are replicated along with the host genome. A skilled person in the art will appreciate that different vectors may be suitable for different delivery methods and have different host tropism, and will be able to select one or more vectors suitable for the use.

### II. PHARMACEUTICAL COMPOSITIONS

Provided herein are compositions (e.g., pharmaceutical compositions) comprising a cell comprising a CAR as disclosed herein.

For therapeutic use, a composition comprising a cell comprising a protein encoded by a transgene as disclosed herein is combined with a pharmaceutically acceptable carrier. The term “pharmaceutically acceptable” as used herein can refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit-to-risk ratio.

The term “pharmaceutically acceptable carrier” as used herein includes buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, PA (1975). Pharmaceutically acceptable carriers include buffers, solvents, dispersion media, coatings, isotonic and absorption delaying agents, or the like, that are compatible with pharmaceutical administration. The use of such media and agents for pharmaceutically active substances is known in the art.

In certain embodiments, a pharmaceutical composition disclosed herein comprises a salt, e.g., NaCl, MgCl2, KCl, MgSO4, etc.; a buffering agent, e.g., a Tris buffer, N-(2-Hydroxyethyl) piperazine-N′-(2-ethanesulfonic acid) (HEPES), 2-(N-Morpholino) ethanesulfonic acid (MES), MES sodium salt, 3-(N-Morpholino) propanesulfonic acid (MOPS), N-tris [Hydroxymethyl]methyl-3-aminopropanesulfonic acid (TAPS), etc.; a solubilizing agent; a detergent, e.g., a non-ionic detergent such as Tween-20, etc.; a nuclease inhibitor; or the like.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as Pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (see, Remington's Pharmaceutical Sciences, 18th ed. (Mack Publishing Company, 1990).

In certain embodiments, a pharmaceutical composition may contain nanoparticles, e.g., polymeric nanoparticles, liposomes, or micelles (Anselmo (2016) BIOENG TRANSL MED). In certain embodiment, the pharmaceutical composition comprises an inorganic nanoparticle. Exemplary inorganic nanoparticles include, e.g., magnetic nanoparticles (e.g., Fe₃MnO₂) or silica. The outer surface of the nanoparticle can be conjugated with a positively charged polymer (e.g., polyethylenimine, polylysine, polyserine) which allows for attachment (e.g., conjugation or entrapment) of payload. In certain embodiment, the pharmaceutical composition comprises an organic nanoparticle (e.g., entrapment of the payload inside the nanoparticle). Exemplary organic nanoparticles include, e.g., SNALP liposomes that contain cationic lipids together with neutral helper lipids which are coated with polyethylene glycol (PEG) and protamine and nucleic acid complex coated with lipid coating. In certain embodiment, the pharmaceutical composition comprises a liposome, for example, a liposome disclosed in International (PCT) Application Publication No. WO 2015/148863.

In certain embodiments, the pharmaceutical composition comprises a targeting moiety to increase target cell binding or update of nanoparticles and liposomes. Exemplary targeting moieties include cell specific antigens, monoclonal antibodies, single chain antibodies, aptamers, polymers, sugars, and cell penetrating peptides. In certain embodiments, the pharmaceutical composition comprises a fusogenic or endosome-destabilizing peptide or polymer.

In certain embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly(2-hydroxyethyl-methacrylate), ethylene vinyl acetate, or poly-D(−)-3-hydroxybutyric acid. Sustained release compositions may also include liposomes that can be prepared by any of several methods known in the art.

A pharmaceutical composition of the invention can be administered by a variety of methods known in the art. The route and/or mode of administration vary depending upon the desired results. Administration can be intravenous, intramuscular, intraperitoneal, or subcutaneous, or administered proximal to the site of the target, preferably intravenous. The pharmaceutically acceptable carrier should be suitable for intravenous, intramuscular, subcutaneous, parenteral, spinal, or epidermal administration (e.g., by injection or infusion). Depending on the route of administration, the active compound (e.g., cell comprising a transgene as disclosed herein) may be coated in a material to protect the compound from the action of acids and other natural conditions that may inactivate the active compound.

Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Pharmaceutical formulations preferably are sterile. Sterilization can be accomplished by any suitable method, e.g., filtration through sterile filtration membranes. In certain embodiments, one or more of the components of the composition are filter sterilized become combination with the cell comprising a transgene.

Pharmaceutical compositions of the invention can be prepared in accordance with methods well known and routinely practiced in the art. See, e.g., Remington: The Science and Practice of Pharmacy, Mack Publishing Co., 20th ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Typically, a therapeutically effective dose or efficacious dose of the composition comprising a cell comprising a CAR as disclosed herein is employed in the pharmaceutical compositions of the invention. The compositions disclosed herein are formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for case of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions of the invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions disclosed herein employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

In certain embodiments, composition comprise a dose of at least 5×10⁴, 1×10⁵, 5×10⁵, 1×10⁶, 5×10⁶, 1×10⁷, 5×10⁷, 1×10⁸, 5×10⁸, 1×10⁹, or 2×10⁹ and/or not more than 1×10⁵, 5×10⁵, 1×10⁶, 5×10⁶, 1×10⁷, 5×10⁷, 1×10⁸, 5×10⁸, 1×10⁹, 2×10⁹, or 1×10¹⁰ cells, for example 5×10⁴ to 1×10¹⁰ cells.

In certain embodiments, a second composition further comprises a second therapeutic agent different from the first. The second composition and the first composition (comprising cells) may be the same or they may be different. In certain embodiments, the second therapeutic agent comprises a small molecule, a cell therapy, an antibody, and/or an immunosuppressive agent.

### III. THERAPEUTIC USES

Contemplated herein are uses of a cell as disclosed herein to treat an individual suffering from a disease, such as an autoimmune disease as disclosed herein.

Composition comprising a cell comprising a transgene as disclosed herein can be used to treat a disease or disorder, such as an autoimmune disease, for example intestinal inflammation, e.g., IBD, CD, and/or UC. Accordingly, provided herein is a method of treating a disease or disorder, the method comprising administering to an individual suffering from the disease or disorder an effective amount of a composition comprising a cell disclosed herein, generally a plurality of such cells.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

The terms “treatment”, “treating”, “treat”, “treated”, or the like, as used herein, can refer to obtaining a desired pharmacologic and/or physiologic effect. The effect may be therapeutic in terms of a partial or complete cure for a disease and/or adverse effect attributable to the disease or delaying the disease progression. “Treatment”, as used herein, covers any treatment of a disease in a mammal, e.g., in a human, and includes: (a) inhibiting the disease, i.e., arresting its development; and (b) relieving the disease, i.e., causing regression of the disease. It is understood that a disease or disorder may be identified by genetic methods and treated prior to manifestation of any medical symptom.

For minimization of toxicity and off-target effect, it can be important to control the dosage of cells delivered. Optimal concentrations can be determined by testing different concentrations in a cellular, tissue, or non-human eukaryote animal model. The dose that gives the highest level of desired response while minimizing the level of undesired response is generally selected for ex vivo or in vivo delivery.

In certain embodiments, an immune cell, e.g., a T cell, is modified to express a protein (e.g., a cytokine or an enzyme) that regulates the microenvironment that the immune cell is designed to migrate to (e.g., a tumor microenvironment).

### IV. KIT

It is understood that the cell comprising a transgene as disclosed herein can be packaged in a kit suitable for use by a medical provider. Accordingly, in another aspect, the invention provides kits containing any one or more of the elements disclosed in the above compositions and methods. In certain embodiments, the kit comprises a cell comprising a transgene as disclosed herein and instructions for using the kit. The instructions may be specific to the applications and methods described herein. In certain embodiments, one or more of the elements of the system are provided in a solution. In certain embodiments, one or more of the elements of the system are provided in lyophilized form, and the kit further comprises a diluent. Elements may be provided individually or in combinations, and may be provided in any suitable container, such as a vial, a bottle, a tube, or immobilized on the surface of a solid base (e.g., chip or microarray), In certain embodiments, the kit comprises one or more of the polynucleotides and/or proteins described herein. In certain embodiments, the kit provides all elements of the systems of the invention.

In certain embodiments, a kit further comprises one or more reagents and/or buffers for use in a process utilizing one or more of the elements described herein. Reagents may be provided in any suitable container and may be provided in a form that is usable in a particular assay, or in a form that requires addition of one or more other components before use (e.g., in concentrate or lyophilized form). A buffer may be a reaction or storage buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In certain embodiments, the buffer has a pH from about 7 to about 10. In certain embodiments, the kit further comprises a pharmaceutically acceptable carrier. In certain embodiments, the kit further comprises one or more devices or other materials for administration to a subject.

### V. EMBODIMENTS

In embodiment 1 provided herein is a cell comprising a polynucleotide in a FOXP3 locus comprising a nucleotide sequence comprising a promoter, a FOXP3 gene or a functional derivative thereof, and a transgene, wherein expression of the FOXP3 gene or functional derivative thereof and the transgene are under the control of the promoter.

In embodiment 2 provided herein is the cell of embodiment 1, wherein the nucleotide sequence further comprises a heterologous 3′ untranslated region (UTR).

In embodiment 3 provided herein is the cell of embodiment 1 or embodiment 2, wherein the promoter is a FOXP3 promoter.

In embodiment 4 provided herein is the cell of any one of embodiments 1-3, wherein the FOXP3 locus comprises a safe harbor site.

In embodiment 5 provided herein is the cell of any one of embodiments 1-4, wherein the transgene is inserted upstream of a start codon of the FOXP3 gene.

In embodiment 6 provided herein is the cell of embodiment 5, wherein the transgene is inserted about 61 to 1 bases before the start codon of the FOXP3 gene.

In embodiment 7 provided herein is the cell of embodiment 6, wherein the transgene is inserted immediately upstream of the start codon of the FOXP3 gene.

In embodiment 8 provided herein is the cell of any one of embodiments 1-4, wherein the transgene is inserted downstream of the FOXP3 gene.

In embodiment 9 provided herein is the cell of embodiment 8, wherein the transgene is inserted about 61 to 1 bases after the stop codon of the FOXP3 gene.

In embodiment 10 provided herein is the cell of embodiment 9, wherein the transgene is inserted immediately after the stop codon of the FOXP3 gene.

In embodiment 11 provided herein is the cell of any one of embodiments 8-10, wherein the stop codon of the FOXP3 gene is engineered to encode an amino acid or is deleted.

In embodiment 12 provided herein is the cell of any one of embodiments 1-11, wherein the FOXP3 gene encodes a full length foxp3 polypeptide.

In embodiment 13 provided herein is the cell of any one of embodiments 1-12, wherein the nucleotide sequence further encodes a self-cleaving peptide between the FOXP3 gene and the transgene.

In embodiment 14 provided herein is the cell of embodiment 13, wherein the self-cleaving peptide (SCP) comprises a 2A peptide.

In embodiment 15 provided herein is the cell of embodiment 14, wherein the 2A peptide comprises a P2A, E2A, F2A, or T2A peptide.

In embodiment 16 provided herein is the cell of any one of embodiments 1-15, wherein the FOXP3 gene and the transgene are expressed as a single polypeptide.

In embodiment 17 provided herein is the cell of embodiment 16, wherein the polypeptide comprises from N- to C-terminus: transgene-SCP-foxp3.

In embodiment 18 provided herein is the cell of embodiment 16, wherein the polypeptide comprises from N- to C-terminus: foxp3-SCP-transgene.

In embodiment 19 provided herein is the cell of embodiment 17 or embodiment 18, further comprising a foxp3 polypeptide comprising a first portion of the self-cleaving peptide and a polypeptide encoded by the transgene comprising a second portion of the self-cleaving peptide.

In embodiment 20 provided herein is the cell of any one of embodiments 1-12, wherein the FOXP3 gene and the transgene are expressed as separate polypeptides.

In embodiment 21 provided herein is the cell of embodiment 20, wherein the nucleotide sequence further comprises an internal ribosome entry site (IRES) between the FOXP3 gene and the transgene.

In embodiment 22 provided herein is the cell of any one of embodiments 1-21, wherein the transgene (TG) encodes a cytokine, interleukin, cytokine receptor, transcription factor, and/or chimeric receptor.

In embodiment 23 provided herein is the cell of embodiment 22, wherein the transgene encodes a chimeric antigen receptor (CAR) or CAR component and/or a T-cell receptor (TCR).

In embodiment 24 provided herein is the cell of embodiment 22 or 23, wherein the transgene encodes a chemokine receptor.

In embodiment 25 provided herein is the cell of embodiment 24, wherein the chemokine receptor (CR) comprises a CCR or CXCR receptor.

In embodiment 26 provided herein is the cell of embodiment 25, wherein the CCR receptor comprises CCR7 and/or CCR9.

In embodiment 27 provided herein is the cell of any one of embodiments 24-26, wherein the polypeptide expressed from the FOXP3 locus comprises from N- to C-terminus: TG-SCP-foxp3.

In embodiment 28 provided herein is the cell of embodiment 27, wherein the polypeptide expressed from the FOXP3 locus comprises from N- to C-terminus: CAR-SCP1-TG-SCP2-foxp3.

In embodiment 29 provided herein is the cell of embodiment 28, wherein the wherein the polypeptide expressed from the FOXP3 locus comprises from N- to C-terminus: CAR-SCP1-CR-SCP2-foxp3.

In embodiment 30 provided herein is the cell of embodiment 28 or 29, wherein the SCP1 and SCP2 are different.

In embodiment 31 provided herein is the cell of any one of embodiments 2-30, wherein the heterologous 3′ UTR comprises one or more of the following 3′ UTRs (1) SV40 reversed, (ii) bGH, (iii) WPRE, (iv) WPRE3/SV40p128, (v) SV40 corrected, (vi) WPRE3/SV40.2, (vii) TCRA EIF3, or (viii) s27a3RU.

In embodiment 32 provided herein is the cell of embodiment 31, wherein the 3′ UTR comprises a SV40 reversed, SV40 corrected, or bGH 3′UTR.

In embodiment 33 provided herein is the cell of any one of embodiments 1-32, wherein the FOXP3 gene further comprises one or more mutations in a codon encoding a ubiquitination or deubiquitination site.

In embodiment 34 provided herein is the cell of embodiment 33, wherein the ubiquitination or deubiquitination site comprises codon 227, 250, 263, 268, and/or 422.

In embodiment 35 provided herein is the cell of any one of embodiments 1-34, wherein the cell comprises a eukaryotic cell.

In embodiment 36 provided herein is the cell of embodiment 35, wherein the eukaryotic cell comprises a metazoan cell.

In embodiment 37 provided herein is the cell of embodiment 35 or embodiment 36, wherein the eukaryotic cell comprises a human cell.

In embodiment 38 provided herein is the cell of any one of embodiments 35-37, wherein the cell comprises an immune cell or an immune precursor cell.

In embodiment 39 provided herein is the cell of embodiment 38, wherein the immune cell comprises a neutrophil, eosinophil, basophil, mast cell, monocyte, macrophage, dendritic cell, natural killer cell, T cell, or a lymphocyte.

In embodiment 40 provided herein is the cell of embodiment 39, wherein the immune cell comprises a T cell.

In embodiment 41 provided herein is the cell of embodiment 40, wherein the T cell comprises an immunosuppressive T cell.

In embodiment 42 provided herein is the cell of embodiment 41, wherein the immunosuppressive T cell comprises a regulatory T cell (Treg).

In embodiment 43 provided herein is the cell of embodiment 38, wherein the immune precursor cell comprises a stem cell, an induced pluripotent stem cell, a hematopoietic stem cell, or a CD4+ stem cell.

In embodiment 44 provided herein is the cell of any one of embodiments 36-43, wherein the cell is isolated from an individual.

In embodiment 45 provided herein is the cell of embodiment 44, wherein the individual is suffering from a disease.

In embodiment 46 provided herein is the cell of embodiment 45, wherein the disease comprises an autoimmune disease.

In embodiment 47 provided herein is the cell of embodiment 46, wherein the autoimmune disease comprises intestinal inflammation.

In embodiment 48 provided herein is the cell of embodiment 47, wherein the autoimmune disease comprises inflammatory bowel disease (IBD).

In embodiment 49 provided herein is the cell of embodiment 48, wherein the autoimmune disease comprises autoimmune disease comprises Crohn's disease (CD) or ulcerative colitis (UC).

In embodiment 50 provided herein is the cell of embodiment 49, wherein the autoimmune disease comprises CD.

In embodiment 51 provided herein is the cell of embodiment 49, wherein the autoimmune disease comprises UC.

In embodiment 52 provided herein is the cell of embodiment 44, wherein the cell is isolated from a healthy individual.

In embodiment 53 provided herein is the cell of embodiment 52, wherein the cell is engineered to not promote an immune response in an allogeneic individual.

In embodiment 54 provided herein is the cell of any one of embodiments 22-53, further comprising a CAR polypeptide expressed on a surface of the cell.

In embodiment 55 provided herein is the cell of any one of embodiments 25-54, further comprises a CR polypeptide expressed on a surface of the cell.

In embodiment 56 provided herein is a cell comprising a polynucleotide in a FOXP3 locus comprising a nucleotide sequence comprising a promoter and a transgene encoding a chimeric antigen receptor (CAR) and a chemokine receptor (CR), wherein expression of the transgene is under the control of the promoter.

In embodiment 57 provided herein is the cell of embodiment 56, wherein the nucleotide sequence further comprises a heterologous 3′ untranslated region (UTR).

In embodiment 58 provided herein is the cell of embodiment 56 or embodiment 57, wherein the promoter is a FOXP3 promoter.

In embodiment 59 provided herein is the cell of any one of embodiments 56-58, wherein the nucleotide sequence further comprises a FOXP3 gene or a functional derivative thereof, wherein expression of the FOXP3 gene or functional derivative thereof is under the control of the FOXP3 promoter.

In embodiment 60 provided herein is the cell of embodiment 59, wherein the FOXP3 gene encodes a full length foxp3 polypeptide.

In embodiment 61 provided herein is a cell comprising a polynucleotide in a FOXP3 locus comprising a nucleotide sequence comprising a promoter, a FOXP3 gene or a functional derivative thereof, and a heterologous 3′ untranslated region (UTR), wherein expression of the FOXP3 gene or functional derivative thereof is under the control of the promoter.

In embodiment 62 provided herein is the cell of embodiment 61, wherein the promoter comprises a FOXP3 promoter.

In embodiment 63 provided herein is the cell of embodiment 61 or embodiment 62, wherein the FOXP3 gene encodes a full length foxp3 polypeptide.

In embodiment 64 provided herein is the cell of any one of embodiments 61-63, wherein the nucleotide sequence further comprises a transgene, wherein expression of the transgene is under the control of the FOXP3 promoter.

In embodiment 65 provided herein is the cell of embodiment 64, wherein the transgene encodes a chimeric antigen receptor (CAR) or CAR component.

In embodiment 66 provided herein is the cell of embodiment 64 or embodiment 65, wherein the transgene encodes a self-cleaving peptide.

In embodiment 67 provided herein is the cell of any one of embodiments 64-66, wherein the transgene encodes a cytokine, interleukin, cytokine receptor, transcription factor, and/or chimeric receptor.

In embodiment 68 provided herein is the cell of embodiment 67, wherein the transgene encodes a chemokine receptor.

In embodiment 69 provided herein is a cell comprising a polynucleotide in a FOXP3 locus comprising a nucleotide sequence comprising a FOXP3 gene or a functional derivative thereof and a heterologous 5′ and/or 3′ UTR, wherein expression of the FOXP3 or functional derivative thereof is under the control of the promoter.

In embodiment 70 provided herein is the cell of embodiment 69, wherein the FOXP3 gene encodes a full length foxp3 polypeptide.

In embodiment 71 provided herein is the cell of embodiment 69 or embodiment 70, wherein the nucleotide sequence further comprises a transgene, wherein expression of the transgene is under the control of the promoter.

In embodiment 72 provided herein is the cell of embodiment 71, wherein the promoter is a FOXP3 promoter.

In embodiment 73 provided herein is the cell of embodiment 71 or embodiment 72, wherein the transgene (TG) encodes a cytokine, interleukin, cytokine receptor, transcription factor, and/or chimeric receptor.

In embodiment 74 provided herein is the cell of embodiment 73, wherein the transgene encodes a chimeric antigen receptor (CAR) or CAR component.

In embodiment 75 provided herein is the cell of embodiment 73 or embodiment 74, wherein the transgene encodes a chemokine receptor.

In embodiment 76 provided herein is the cell of any one of embodiments 71-73, wherein the transgene encodes a self-cleaving peptide.

In embodiment 77 provided herein is a derivative of or a progeny of a cell of any one of embodiments 1-76.

In embodiment 78. Use of a cell of any one of embodiments 1-76, to treat an individual suffering from a disease.

In embodiment 79 provided herein is the cell of embodiment 77, wherein the disease comprises an autoimmune disease.

In embodiment 80 provided herein is the cell of embodiment 79, wherein the autoimmune disease comprises intestinal inflammation.

In embodiment 81 provided herein is the cell of embodiment 80, wherein the autoimmune disease comprises inflammatory bowel disease (IBD).

In embodiment 82 provided herein is the cell of embodiment 81, wherein the autoimmune disease comprises autoimmune disease comprises Crohn's disease (CD) or ulcerative colitis (UC).

In embodiment 83 provided herein is the cell of embodiment 82, wherein the autoimmune disease comprises CD.

In embodiment 84 provided herein is the cell of embodiment 82, wherein the autoimmune disease comprises UC.

In embodiment 85 provided herein is a composition comprising: (1) a nucleic acid-guided nuclease system comprising: (a) a nucleic acid-guided nuclease, or a polynucleotide encoding the nuclease; and (b) a guide nucleic acid (gNA), or a polynucleotide encoding the gNA, comprising a spacer sequence complementary to a site within or near the FOXP3 gene; and (2) a donor template encoding a transgene.

In embodiment 86 provided herein is the composition of embodiment 85, wherein the site is upstream of a start codon of the FOXP3 gene.

In embodiment 87 provided herein is the composition of embodiment 86, wherein the site is about 61 to 1 bases before the start codon of the FOXP3 gene.

In embodiment 88 provided herein is the composition of embodiment 87, wherein the site is immediately upstream of the start codon of the FOXP3 gene.

In embodiment 89 provided herein is the composition of embodiment 85, wherein the site is downstream of the FOXP3 gene.

In embodiment 90 provided herein is the composition of embodiment 89, wherein the site is about 61 to 1 bases after the stop codon of the FOXP3 gene.

In embodiment 91 provided herein is the composition of embodiment 90, wherein the site is immediately after the stop codon of the FOXP3 gene.

In embodiment 92 provided herein is the composition of any one of embodiments 85-91, wherein the transgene encodes a cytokine, interleukin, cytokine receptor, transcription factor, and/or chimeric receptor.

In embodiment 93 provided herein is the composition of embodiment 92, wherein the transgene encodes a CAR or CAR component and/or a T-cell receptor.

In embodiment 94 provided herein is the composition of embodiment 93, wherein the transgene encodes a CAR or a CAR component.

In embodiment 95 provided herein is the composition of any one of embodiments 92-94, wherein the transgene encodes a cytokine receptor.

In embodiment 96 provided herein is the composition of any one of embodiments 92-95, wherein the transgene encodes a self-cleaving peptide.

In embodiment 97 provided herein is the composition of embodiment 96, wherein the self-cleaving peptide comprises a 2A peptide.

In embodiment 98 provided herein is the composition of embodiment 97, wherein the 2A peptide comprises a P2A, E2A, F2A, or T2A peptide.

In embodiment 99 provided herein is the composition of any one of embodiments 85-98, wherein the spacer sequence comprises any one of SEQ ID NOs: 1-14.

In embodiment 100 provided herein is the composition of any one of embodiments 85-99, wherein the nucleic acid-guide nuclease comprises a Class 1 or Class 2 nuclease.

In embodiment 101 provided herein is the composition of embodiment 100, wherein the nucleic acid-guided nuclease comprises a Type II or Type V nuclease.

In embodiment 102 provided herein is the composition of embodiment 101, wherein the nucleic acid-guided nuclease comprises cas9.

In embodiment 103 provided herein is the composition of embodiment 101, wherein the nucleic acid-guided nuclease comprises a Type V-A, V-B, V-C, V-D, or V-E nuclease.

In embodiment 104 provided herein is the composition of any one of embodiments 85-103, wherein the nuclease comprises one or more nuclear localization signals.

In embodiment 105 provided herein is the composition of any one of embodiments 85-104, wherein the gNA comprises a gRNA.

In embodiment 106 provided herein is the composition of any one of embodiments 85-105, wherein nucleic acid-guided nuclease complex and the gNA are present as a nucleic acid-guided nuclease complex.

In embodiment 107 provided herein is a cell comprising a composition of any one of embodiments 85-106, or a derivative or progeny thereof.

In embodiment 108 provided herein is the cell of 107, wherein the cell comprises a eukaryotic cell.

In embodiment 109 provided herein is the cell of embodiment 108, wherein the eukaryotic cell comprises a metazoan cell.

In embodiment 110 provided herein is the cell of embodiment 108 or embodiment

109, wherein the eukaryotic cell comprises a human cell.

In embodiment 111 provided herein is the cell of any one of embodiments 107-110, wherein the cell comprises an immune cell or an immune precursor cell.

In embodiment 112 provided herein is the cell of embodiment 111, wherein the immune cell comprises a neutrophil, eosinophil, basophil, mast cell, monocyte, macrophage, dendritic cell, natural killer cell, T cell, or a lymphocyte.

In embodiment 113 provided herein is the cell of embodiment 112, wherein the immune cell comprises a T cell.

In embodiment 114 provided herein is the cell of embodiment 113, wherein the T cell comprises an immunosuppressive T cell.

In embodiment 115 provided herein is the cell of embodiment 114, wherein the immunosuppressive T cell comprises a regulatory T cell (Treg).

In embodiment 116 provided herein is the cell of embodiment 111, wherein the immune precursor cell comprises a stem cell, an induced pluripotent stem cell, a hematopoietic stem cell, or a CD4+ stem cell.

In embodiment 117 provided herein is the cell of any one of embodiments 109-116, wherein the cell is isolated from an individual.

In embodiment 118 provided herein is the cell of embodiment 117, wherein the individual is suffering from a disease.

In embodiment 119 provided herein is the cell of embodiment 118, wherein the disease comprises an autoimmune disease.

In embodiment 120 provided herein is the cell of embodiment 119, wherein the autoimmune disease comprises intestinal inflammation.

In embodiment 121 provided herein is the cell of embodiment 120, wherein the autoimmune disease comprises inflammatory bowel disease (IBD).

In embodiment 122 provided herein is the cell of embodiment 121, wherein the autoimmune disease comprises autoimmune disease comprises Crohn's disease (CD) or ulcerative colitis (UC).

In embodiment 123 provided herein is the cell of embodiment 122, wherein the autoimmune disease comprises CD.

In embodiment 124 provided herein is the cell of embodiment 122, wherein the autoimmune disease comprises UC.

In embodiment 125 provided herein is the cell of embodiment 117, wherein the cell is isolated from a healthy individual.

In embodiment 126 provided herein is the cell of embodiment 125, wherein the cell is engineered to not promote an immune response in an allogeneic individual.

In embodiment 127. Use of a composition of any one of embodiments 85-106 to generate a cell of any one of embodiments 1-84.

In embodiment 128 provided herein is a method for editing a genome of a cell

comprising contacting the cell with a composition of any one of embodiments 85-106, whereby the resulting cell comprises a heterologous polynucleotide in a FOXP3 locus.

In embodiment 129 provided herein is the method of 128, wherein the cell comprises a eukaryotic cell.

In embodiment 130 provided herein is the method of embodiment 129, wherein the eukaryotic cell comprises a metazoan cell.

In embodiment 131 provided herein is the method of embodiment 129 or embodiment 130, wherein the eukaryotic cell comprises a human cell.

In embodiment 132 provided herein is the method of any one of embodiments 128-131, wherein the cell comprises an immune cell or an immune precursor cell.

In embodiment 133 provided herein is the method of embodiment 132, wherein the immune cell comprises a neutrophil, eosinophil, basophil, mast cell, monocyte, macrophage, dendritic cell, natural killer cell, T cell, or a lymphocyte.

In embodiment 134 provided herein is the method of embodiment 133, wherein the immune cell comprises a T cell.

In embodiment 135 provided herein is the method of embodiment 134, wherein the T cell comprises an immunosuppressive T cell.

In embodiment 136 provided herein is the method of embodiment 135, wherein the immunosuppressive T cell comprises a regulatory T cell (Treg).

In embodiment 137 provided herein is the method of embodiment 132, wherein the immune precursor cell comprises a stem cell, an induced pluripotent stem cell, a hematopoietic stem cell, or a CD4+ stem cell.

In embodiment 138 provided herein is the method of any one of embodiments 128-137, wherein, after editing, expression of foxp3 and/or the transgene in the cell is at least 1.1, 1.2, 1.3, 1.4, 1.5, 1.6, 1.7, 1.8, 1.9, 2, 2.2, 2.4, 2.6, 2.8, 3, 3.5, 4, or 4.5-fold and/or not more than 1.2, 1.3, 1.4, 1.5, 1.6, 1.7, 1.8, 1.9, 2, 2.2, 2.4, 2.6, 2.8, 3, 3.5, 4, 4.5, or 5-fold greater than a cell comprising the wildtype FOXP3 3′ UTR, for example 1.1 to 5-fold, preferably 1.1 to 3-fold, more preferably 1.5 to 2-fold.

In embodiment 139 provided herein is the method of any one of embodiments 128-138, wherein, after editing, the cell demonstrates no more than 50, 40, 30, 20, 10, 9, 8, 7, 6, 5, 4, 3, 2, 1 or, 0% expression of a transgene within FOXP3 when measured as an effector T-cell as compared to when measured as an immunosuppressive T-cell.

In embodiment 140 provided herein is a composition comprising a cell generated with a method of any one of embodiments 128-139, a derivative thereof, or a progeny thereof. In embodiment 141 provided herein is a method of editing genomes of a plurality of

cells comprising contacting the plurality with a composition of any one of embodiments 85-106, whereby the resulting cell comprises a heterologous polynucleotide in FOXP3.

In embodiment 142 provided herein is the method of embodiment 141, wherein the plurality of cells are isolated from an individual.

In embodiment 143 provided herein is the method of any one of embodiments 141 or embodiment 142, wherein isolating further comprise purifying the cells such that the plurality of cells comprises at least 50, 60, 70, 80, 85, 90, 95, 96, 97, 98, 99, 99.5, or 99.9% immunosuppressive T-cells.

In embodiment 144 provided herein is the method of any one of embodiments 141-143, wherein, after editing, expression of foxp3 and/or the transgene in the cell is at least 1.1, 1.2, 1.3, 1.4, 1.5, 1.6, 1.7, 1.8, 1.9, 2, 2.2, 2.4, 2.6, 2.8, 3, 3,5, 4, or 4.5-fold and/or not more than 1.2, 1.3, 1.4, 1.5, 1.6, 1.7, 1.8, 1.9, 2, 2.2, 2.4, 2.6, 2.8, 3, 3.5, 4, 4.5, or 5-fold greater than a cell comprising the wildtype FOXP3 3′ UTR, for example 1.1 to 5-fold, preferably 1.1 to 3-fold, more preferably 1.5 to 2-fold.

In embodiment 145 provided herein is the method of any one of embodiments 141-144, wherein, after editing, the cell demonstrates no more than 50, 40, 30, 20, 10, 9, 8, 7, 6, 5, 4, 3, 2, 1 or, 0% expression of a transgene within the FOXP3 locus when measured as an effector T-cell as compared to when measured as an immunosuppressive T-cell.

In embodiment 146 provided herein is a composition comprising a plurality of cells generated with a method of any one of embodiments 141-145.

In embodiment 147 provided herein is the composition of embodiment 146, wherein the plurality of cells comprises at least 80, 85, 90, 95, 96, 97, 98, 99, or 100% of all of the cells in the composition.

In embodiment 148 provided herein is a composition comprising: (1) a nucleic acid-guided nuclease system comprising: (a) a nucleic acid-guided nuclease, or a polynucleotide encoding the nuclease; and (b) a guide nucleic acid (gNA) comprising a nucleotide sequence at least 80, 85, 90, 95, 96, 97, 98, 99, or 100% identical to any one of SEQ ID NOs: 1-14; and (2) a donor template encoding a transgene.

### VI. EXAMPLES

### A. Example 1: Insertion of a Transgene Upstream of a FOXP3 Gene

In this example, a transgene is inserted upstream of a FOXP3 gene such that both the transgene and the FOXP3 gene (201) are under the control of a promoter (206) and the transgene and the FOXP3 gene are expressed as a single polypeptide. While any suitable method can be used to deliver the transgene to the FOXP3 locus, this method envisions use of a nucleic acid-guided nuclease system, e.g., a CRISPR-cas system. This method is illustrated in FIG. 2.

A polynucleotide comprising a transgene (202), a self-cleaving peptide (203), an upstream homology arm (204) and a downstream homology arm (205) are delivered to an immunosuppressive cell along with a nucleic acid-guided nuclease system comprising a nucleic acid-guide nuclease and a guide nucleic acid (gNA), or one or more polynucleotides encoding the nucleic acid-guided nuclease system. The gNA comprises a spacer sequence complementary to a site upstream of the start codon of the FOXP3 gene (201), such that when the nucleic acid-guide nuclease system binds to the site, one or more strand breaks is generated at or near the site. The upstream homology arm (204) is at least partially complementary to a region upstream of the one or more strand breaks, and the downstream homology arm (205) is at least partially complementary to a region downstream of the one or more strand breaks. The one or more strand breaks is repaired by homology directed repair using at least a portion of the polynucleotide resulting in the edited FOXP3 locus (207).

When the promoter (206) is activated, a single polypeptide comprising the polypeptide encoded by the transgene, the self-cleaving peptide, and the foxp3 polypeptide are translated. Once sufficiently folded, the self-cleaving peptide cleaves, resulting in a polypeptide encoded by the transgene and a foxp3 polypeptide.

### B. Example 2: Insertion of a Transgene Downstream of a FOXP3 Gene

In this example, a transgene is inserted downstream of a FOXP3 gene such that both the transgene and the FOXP3 gene (301) are under the control of a promoter (306) and the transgene and the FOXP3 gene are expressed as a single polypeptide. While any suitable method can be used to deliver the transgene to the FOXP3 locus, this method envisions use of a nucleic acid-guided nuclease system, e.g., a CRISPR-cas system. This method is illustrated in FIG. 3.

A polynucleotide comprising a transgene (302), a self-cleaving peptide (303), an upstream homology arm (304) and a downstream homology arm (305) are delivered to an immunosuppressive cell along with a nucleic acid-guided nuclease system comprising a nucleic acid-guide nuclease and a guide nucleic acid (gNA), or one or more polynucleotides encoding the nucleic acid-guided nuclease system. The gNA comprises a spacer sequence complementary to a site downstream of the stop codon of the FOXP3 gene (301), such that when the nucleic acid-guide nuclease system binds to the site, one or more strand breaks is generated at or near the site. The upstream homology arm (304) is at least partially complementary to a region upstream of the one or more strand breaks, and the downstream homology arm (305) is at least partially complementary to a region downstream of the one or more strand breaks. The one or more strand breaks is repaired by homology directed repair using at least a portion of the polynucleotide resulting in the edited FOXP3 locus (307).

When the promoter (306) is activated, a single polypeptide comprising the foxp3 polypeptide, the self-cleaving peptide, and the polypeptide encoded by the transgene are translated. Once sufficiently folded, the self-cleaving peptide cleaves, resulting in a foxp3 polypeptide and a polypeptide encoded by the transgene.

### C. Example 3: Selective Expression of Payload in Treg Cells

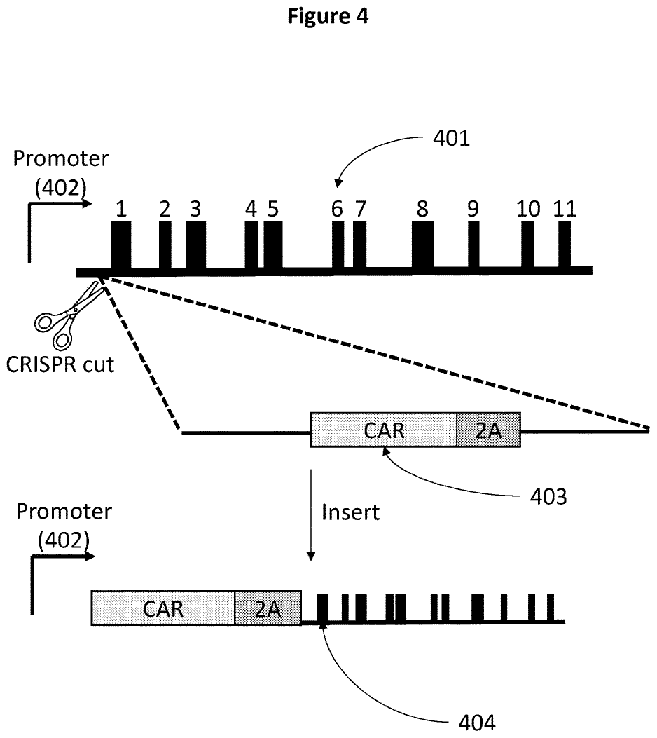

This example demonstrates the ability to preferentially express transgenes in Treg cells when the transgenes are placed under the control of a FOXP3 promoter. FIG. 4 shows an illustration of insertion of a portion of a donor template (403) comprising a CAR and a 2A peptide into the FOXP3 locus (401) under the control of a FOXP3 promoter (403). The resulting polynucleotide (404) comprising a start codon, a CAR, a 2A polypeptide, a nucleotide sequence encoding a foxp3 polypeptide, and a stop codon.

Site-specific integration at FOXP3 was achieved with optimized guides at each site. Primary Treg cells or Jurkat Teff cells were edited at Day 3, 8, or 9 post-isolation by nucleofection with Cas9: Guide RNP, recovered in 500 μL RPMI+10% FBS or OpTmizer media (with 300 IU/mL IL-2) and subsequently transduced with AAV6 containing each donor template at 5% by volume. CTS CD3/CD28 stimulation beads were returned to culture post-editing, and the day after editing cultures containing AAV were diluted 1:1 in fresh media (addition of 500 μL) containing 300 IU/mL IL-2. Functional impact of editing was assessed by Flow cytometry. Briefly edited primary Tregs (performed as described in previous examples) were stained, fixed and permeabilized, then stained for intracellular phenotypic markers such as FOXP3 and HELIOS. Impact on relative % expression, as well as mean fluorescence intensity (MFI), were considered when selecting optimal guides. Cells were fixed and permeabilized using eBiosciences FoxP3 transcription factor buffer set (eBiosciences). Flow analysis on D14 was done with a staining panel including: Aqua Live/Dead, FMC-63-FITC, FOXP3-eF450, Helios-APC and run on the Novocyte. Analysis performed with FlowJo v10.8. Briefly, cells were counted and moved to a round-bottom 96WP. Cells were spun (2000 rpm 3 min) and washed in PBS, stained with 1:1000 Aqua Live/Dead (15 min RT) in the dark, washed 1× with PBS and 1× with Flowstain Buffer (Invitrogen), then incubated with FITC-FMC-63 in Flowstain buffer (15 min RT), washed 2× in Flowstain buffer subsequently Fix/Permeabilized (45 min RT), washed 2× with Perm/wash, and stained with FOXP3-eF450/HELIOS-APC (30 min RT), washed, and resuspended in Flowstain buffer before analysis on the Novocyte. HDR editing was evaluating by flow cytometry by staining with FMC-63-FITC antibody to evaluate CD19CAR expression. For ddPCR measurements, genomic DNA was isolated from edited cells with Qiagen Dneasy Blood and tissue kit, quality was assessed on the nanodrop. ddPCR was run with custom primers (positioned outside the 5′ homology arm and within the inserted construct for our edited amplicon, and another distal locus within FOXP3 for the reference) and probes (FAM for insert and HEX for reference) were ordered from Azenta and IDT respectively, droplets were generated, and samples were amplified using a custom thermocycling profile fit for large amplicons. Fluorescence was detected by the digital droplet reader following manufacturer's recommendations.

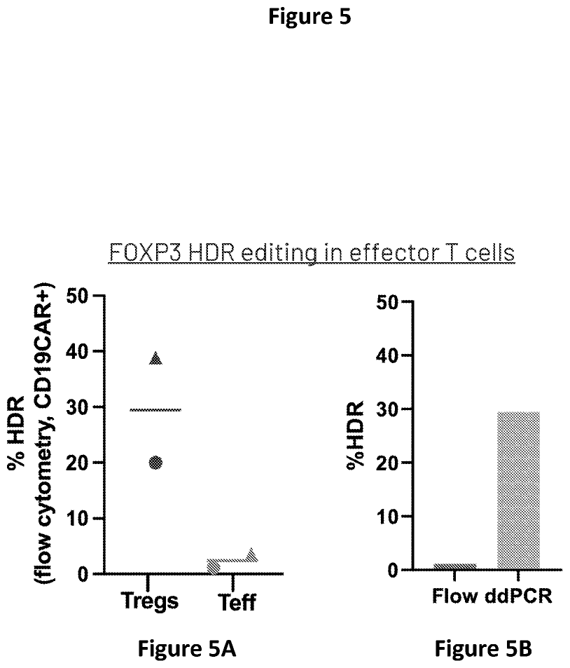

FIG. 5 shows flow cytometry data of the percentage of Treg and Teff cells expressing CAR after insertion of a CAR transgene into the foxp3 locus (Figure SA) and droplet digital PCR data (ddPCR) of the HDR efficiency of the CAR template into the Teff cells (50B. Specifically, Figure SA shows that cells comprising a CAR transgene in the FOXP3 locus under the control of a FOXP3 promoter are preferentially expressed in Treg cells as compared to Teff cells. 50B shows that even though the Teff cells comprise the CAR transgene as measured by ddPCR, there is little CAR transgene expression in Teff cells due to limited activation of the FOXP3 promoter.

This example demonstrates the ability to selectively express transgenes in Treg cells when the transgenes are placed under the control of a FOXP3 promoter.

### D. Example 4: Identification of Guide Sequences for Insertion of Transgenes in the FOXP3 Locus

This example demonstrates the ability to modify multiple loci within the FOXP3 locus and CAR insertion into the FOXP3 locus under the control of a FOXP3 promoter.

Site-specific integration at FOXP3 was achieved with optimized guides at each site. Primary Treg cells were edited at Day 3, 8, or 9 post-isolation by nucleofection with Cas9: Guide RNP, recovered in 500 μL RPMI+10% FBS or OpTmizer media (with 300 IU/mL IL-2). Afterward, genomic DNA was isolated and amplified by site-specific PCR for sanger sequencing and subsequent indel analysis by Synthego ICE platform. Functional impact of editing on FOXP3 expression was assessed by Flow cytometry. Briefly edited primary Tregs were stained, fixed and permeabilized, then stained for intracellular phenotypic markers such as FOXP3 and HELIOS. Cells were fixed and permeabilized using eBiosciences FoxP3 transcription factor buffer set (eBiosciences).

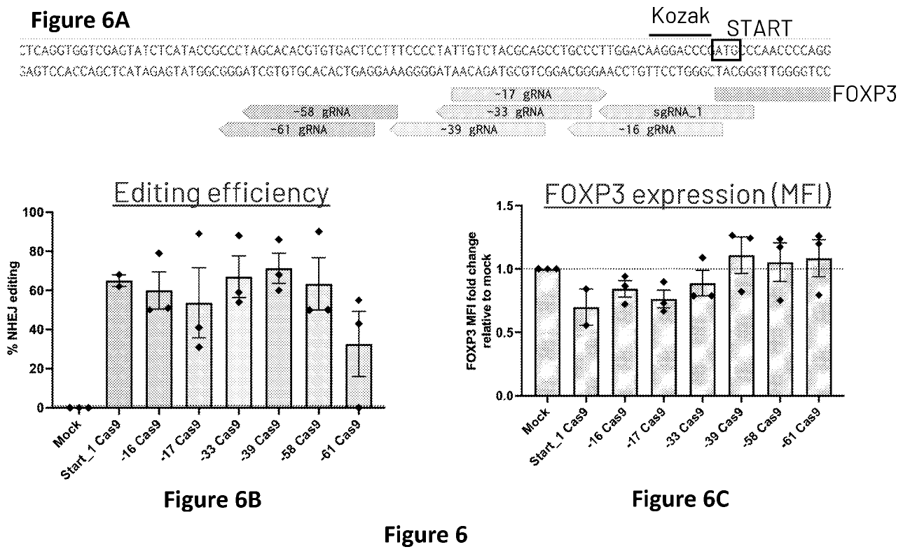

FIG. 6 shows NHEJ editing efficiency (FIG. 6B) and FOXP3 expression (FIG. 6C) after editing with various guide sequences (FIG. 6A). The guides demonstrate a high proportion of NHEJ edits at the target site in primary human Treg cells across multiple donors.

Two guide sequences ‘-33’ (comprising spacer sequence of SEQ ID NO: 10) and ‘-39’ (comprising spacer sequence of SEQ ID NO: 8) were used for FOXP3 HDR editing in Tregs. Cells were edited as described above. Functional impact of editing on FOXP3 expression was assessed by Flow cytometry and HDR efficiency was measured by flow cytometry for CD19 CAR expression. Primary Treg cells were edited at Day 8 post-isolation by nucleofection with Cas9: Guide RNP, recovered in 500 μL OpTmizer media (with 3001U/mL IL-2) and subsequently transduced with AAV6 containing each donor template at 5% by volume. CTS CD3/CD28 stimulation beads were returned to culture post-editing, and the day after editing cultures containing AAV were diluted 1:1 in fresh media (addition of 500 μL) containing 300 IU/mL IL-2.

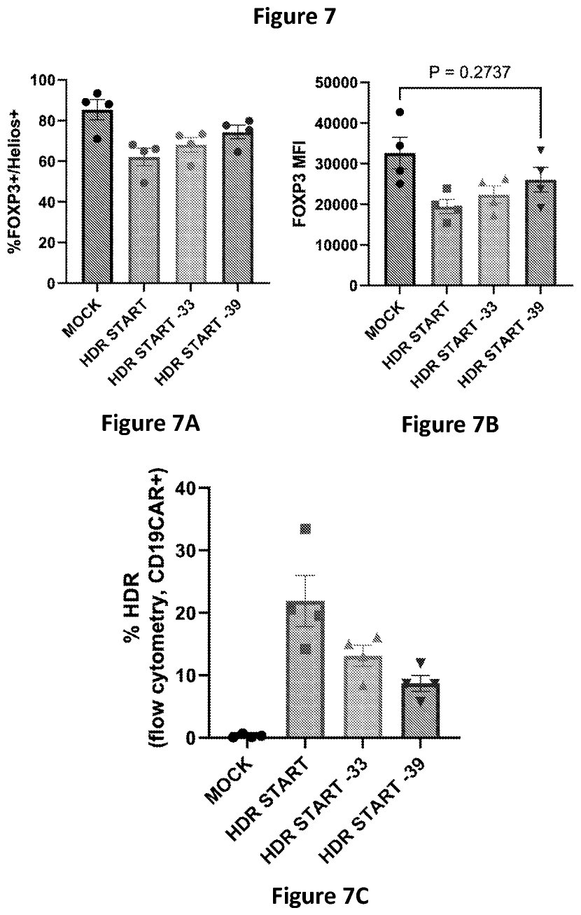

FIG. 7 shows (FIG. 7A) % FOXP3+HELIOS+ cells after editing, (FIG. 7B) FOXP3 expression after editing, and (FIG. 7C) % HDR of CAR after editing. High levels of editing were seen with HDR start, HDR START-33, and HDR START-39 guide RNAs.

This example demonstrates the ability to modify multiple loci within the FOXP3 locus and CAR insertion into the FOXP3 locus under the control of a FOXP3 promoter.

### E. Example 5: Stabilization of FOXP3 and Transgene Expression

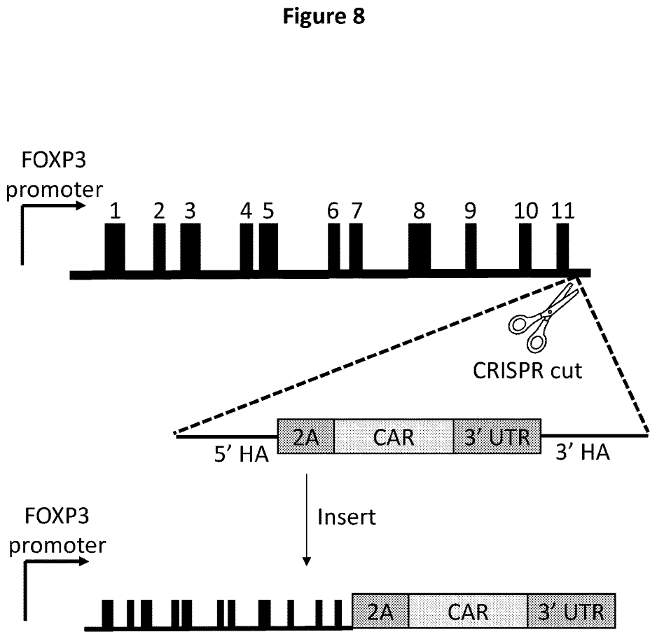

This example demonstrates the stabilization of FOXP3 expression by insertion of heterologous 3′ UTR sequences. Incorporation of 3′ UTRs regulatory element was achieved by HDR editing at the FOXP3 STOP site as shown in FIG. 8. Briefly, a donor template comprising a 2A, CAR, and a heterologous 3′ UTR was inserted downstream of the FOXP3 gene in the FOXP3 locus resulting in a polynucleotide comprising a FOXP3 promoter, a start codon, a nucleic acid sequence encoding a foxp3 polypeptide, a 2A peptide, a CAR, a stop codon, and a 3′ UTR. Incorporation of 3′ UTRs regulatory element increase both FOXP3 and CD19CAR expression. HDR editing was performed as previously described for HDR editing at the FOXP3 START site.

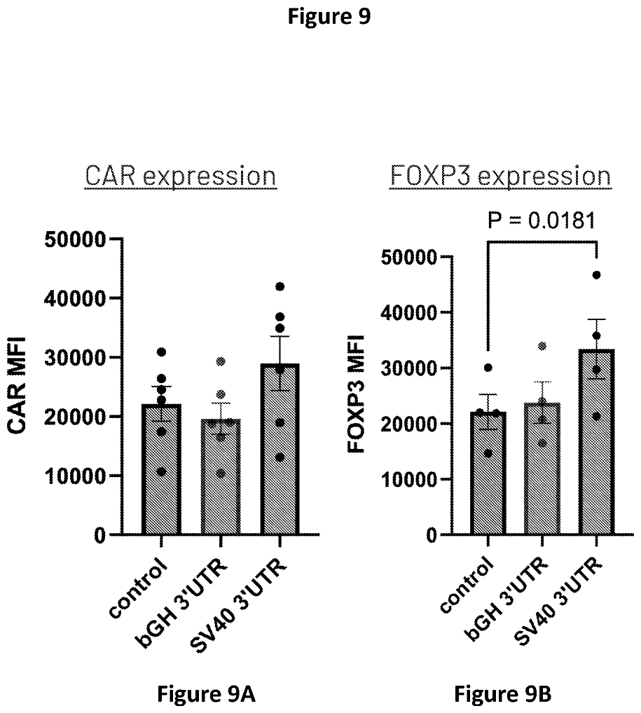

FIG. 9 shows CAR (FIG. 9A) and FOXP3 (FIG. 9B) expression after insertion of bGH or SV40 3′ UTR. The SV40 3′ UTR in particular results in a significant increase in FOXP3 expression as compared to the control.

Incorporation of 3′ UTRs regulatory element increase both FOXP3 and CD19CAR expression. HDR editing was performed as previously described for HDR editing at the FOXP3 START site.

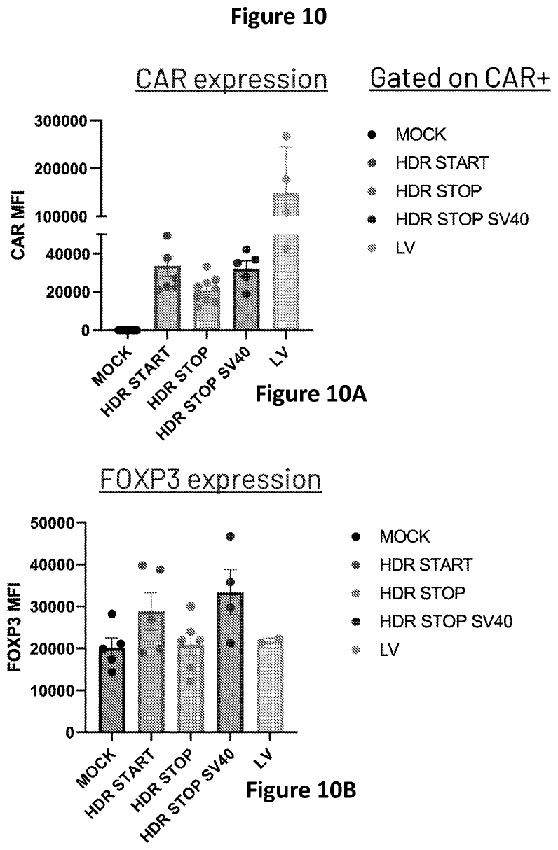

FIG. 10 shows CAR (FIG. 10A) and FOXP3 (FIG. 10B) expression after editing. Specifically edits at the HDR start position or at the HDR stop position with the SV40 3′ UTR result in significant CAR and FOXP3 expression.

This example demonstrates the stabilization of FOXP3 expression by insertion of heterologous 3′ UTR sequences.

### F. Example 6: Antigen-Specific Activation Profiles are Similar Between Engineering Conditions Despite Significant Differential in CAR Expression

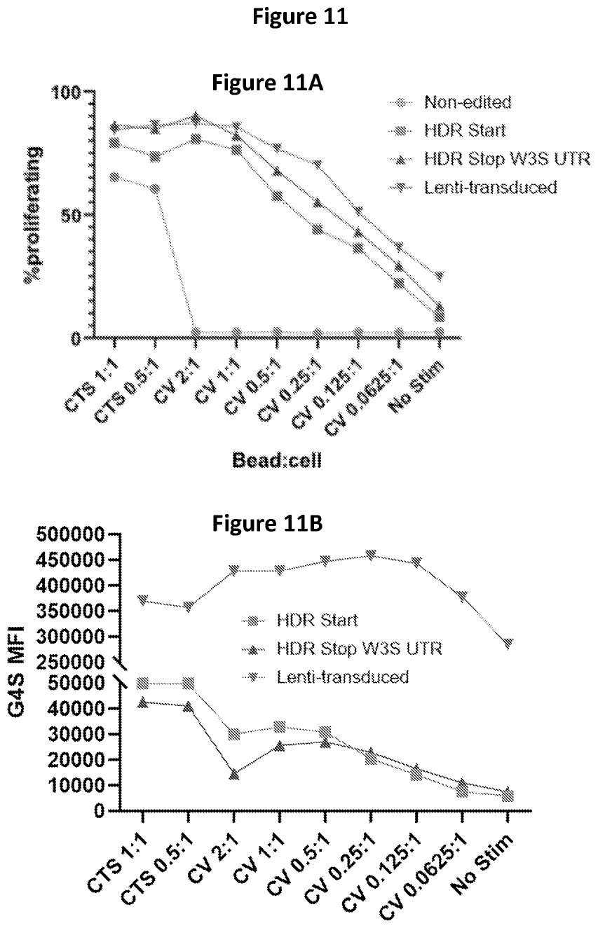

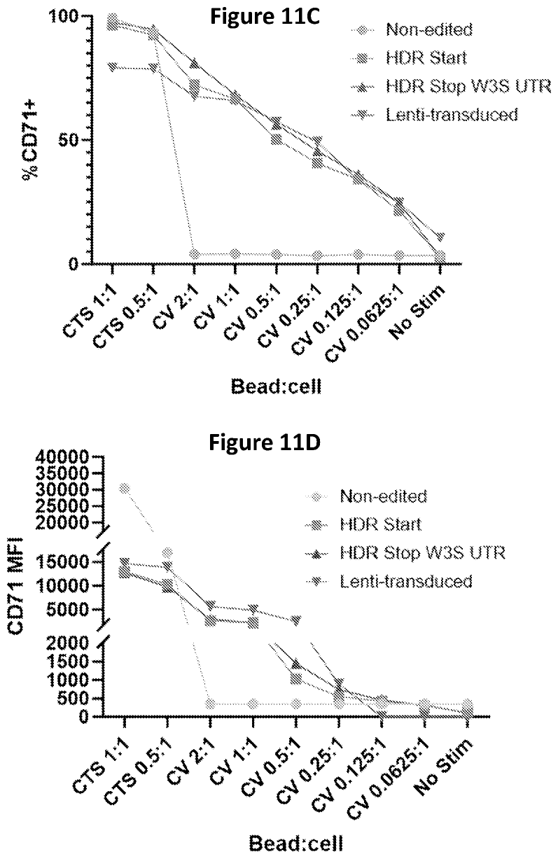

This example demonstrates that Tregs edited by inserting a Citrullinated Vimentin (CV) specific CAR in the FOXP3 locus under the control of a FOXP3 promoter demonstrate similar antigen-specific activation profiles to Tregs where the CV specific CAR was transduced with a lentivirus vector (lenti-transduced). Primary Treg cells edited at Day 3 post-isolation by nucleofection with Cas9: Guide RNP, recovered in 500 μL OpTmizer media (with 300 IU/mL IL-2) and subsequently transduced with AAV6 containing each donor template at 5% by volume. After Day 14 post-isolation cells were switched to RPMI+10% FBS (with 300 IU/mL IL-2) and rested for 3 days. Lentiviral transduced cells were transduced at an MOI of 12 on day 3. On Day 17 post-isolation, cells were CFSE labeled and plated in 96 well plates (100,000 cells/well) and co-incubated with no beads, anti-CD3/28 dynabeads, or anti-CV CAR beads at varying ratios for 3 days before reading out proliferation (FIG. 11A), CAR expression as measured by G4S linker presence (FIG. 11B), and by CFSE and activation by CD71 using flow cytometry (FIGS. 11C and D, respectively).

FIG. 11A shows that % proliferating (y-axis) of HDR start and HDR stop W3S UTR engineered cells demonstrate similar profiles to both non-edited and Lenti-transduced cells when incubated with anti-CD3/28 dynabeads and similar profiles to Lenti-transduced cells when stimulated with anti-CV CAR beads at varying ratios.

FIG. 11B shows that CAR expression as measured by G4S linker MFI (y-axis) is present in both HDR start and HDR stop W3S UTR engineered cells.

FIG. 11C shows that % CD71+ cells (y-axis) for HDR start and HDR stop W3S UTR engineered cells demonstrate similar profiles to both non-edited and Lenti-transduced cells when incubated with anti-CD3/28 dynabeads and similar profiles to Lenti-transduced cells when stimulated with anti-CV CAR beads at varying ratios.

FIG. 11D shows that CD71 MFI (y-axis) for HDR start and HDR stop W3S UTR engineered cells demonstrate similar profiles to both non-edited and Lenti-transduced cells when incubated with anti-CD3/28 dynabeads and similar profiles to Lenti-transduced cells when stimulated with anti-CV CAR beads at varying ratios.

This example demonstrates that cells with a CAR insertion in the FOXP3 locus under the control of a FOXP3 promoter demonstrate similar antigen-specific activation profiles to Lenti-transduced cells.

### G. Example 7: Gene Edited Teff Show Minimal Response to CAR Specific Stimulation while LV Teff Respond Robustly to CAR Specific Stimulation

This example demonstrates that effector T cells (Teff) edited by inserting a CV specific CAR in the FOXP3 locus under the control of a FOXP3 promoter show little to no CAR specific stimulation.

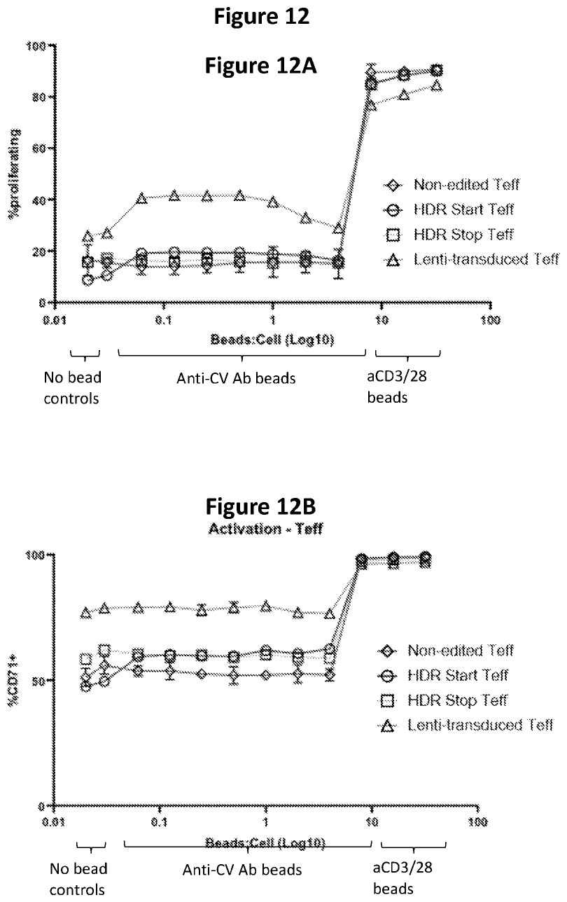

Primary Teff cells edited at Day 3 post-isolation by nucleofection with Cas9: Guide RNP, recovered in 500 μL OpTmizer media (with 300 IU/mL IL-2) and subsequently transduced with AAV6 containing each donor template at 5% by volume. After Day 14 post-isolation cells were switched to RPMI+10% FBS (with 300 IU/mL IL-2) and rested for 3 days. Lentiviral transduced cells were transduced at an MOI of 12 on day 3. On Day 17 post-isolation, cells were CFSE labeled and plated in 96 well plates (100,000 cells/well) and co-incubated with no beads, anti-CD3/28 dynabeads, or anti-CV CAR beads at varying ratios for 3 days before reading out proliferation (FIG. 12A) and by CFSE and activation by CD71 using flow cytometry (FIG. 12B).

FIG. 12A shows that % proliferating (y-axis) of Teff cells engineered with HDR start or HDR stop are similar to non-engineered Teff cells when incubated with Anti-CV Ab beads. Additionally, when incubated with anti-CD3/28 dynabeads, HDR start or HDR stop engineered Teff cells demonstrate similar % proliferating to non-engineered Teff cells.

FIG. 12B shows that % CD71+ (y-axis) of Teff cells engineered with HDR start or HDR stop are similar to non-engineered Teff cells when incubated with Anti-CV Ab beads. Additionally, when incubated with anti-CD3/28 dynabeads, HDR start or HDR stop engineered Teff cells demonstrate similar % proliferating to non-engineered Teff.

This example demonstrates that Toff cells comprising CAR under the control of a FOXP3 promoter show little to no CAR activation.

### H. Example 8: FOXP3 Start Edited CAR-Tregs Efficiently Suppress Allogeneic and Autologous T-Responders in In-Vitro Suppression Assays

This example demonstrates that FOXP3 Start edited CAR-Tregs efficiently suppress allogeneic and autologous T-responders in in-vitro suppression assays.

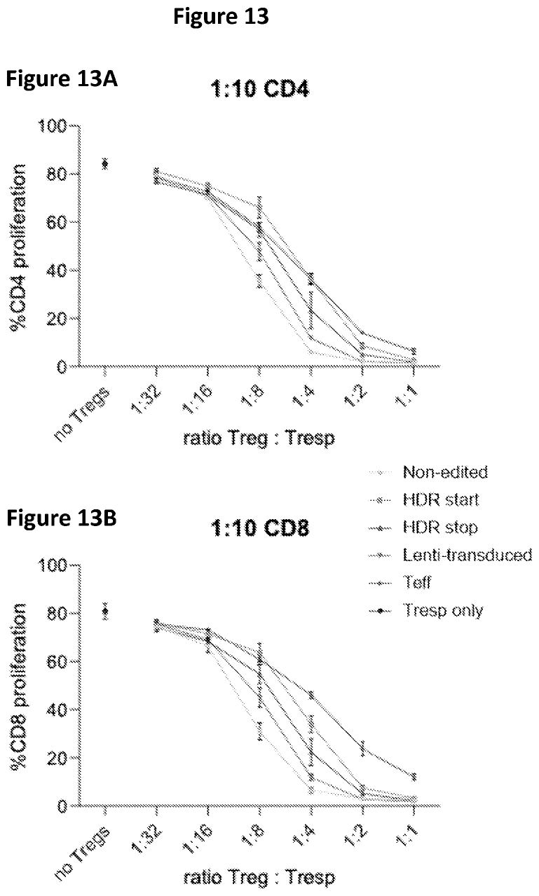

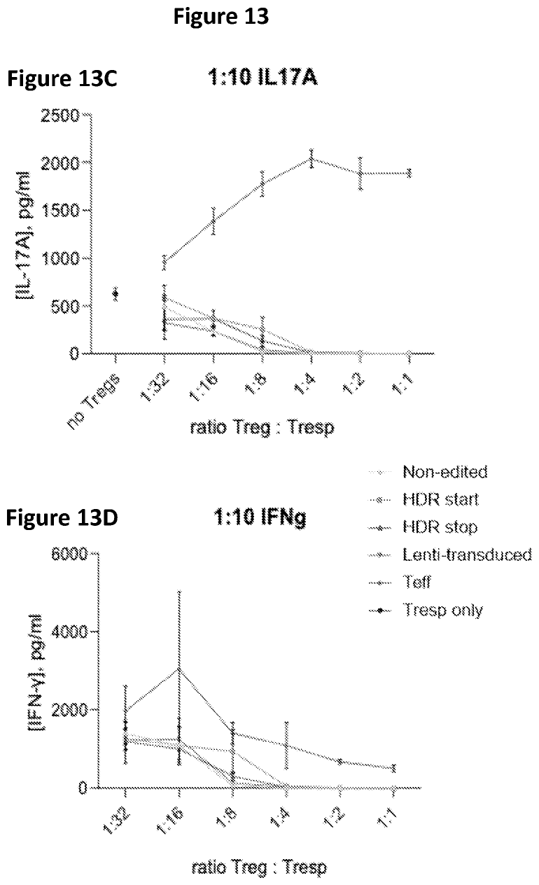

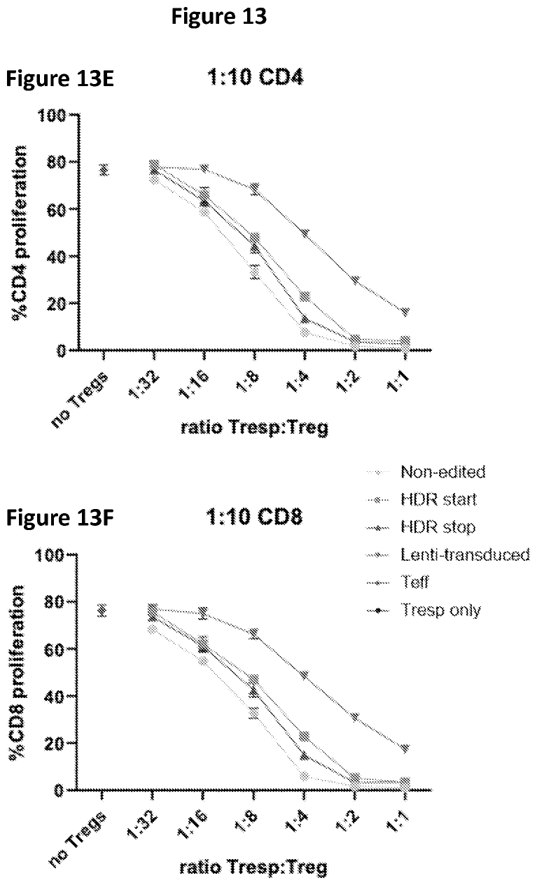

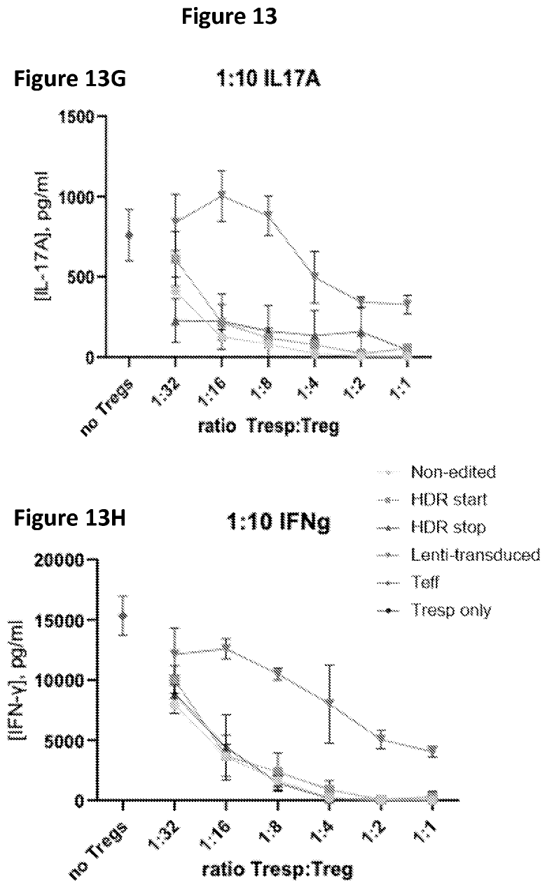

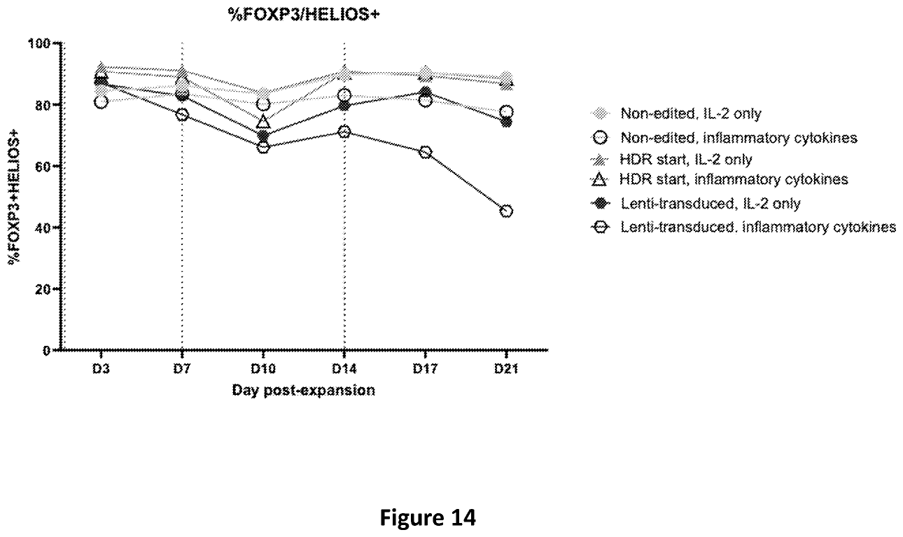

Primary Treg cells were edited at Day 3 post-isolation by nucleofection with Cas9: Guide RNP, recovered in 500¬μL OpTmizer media (with 300 IU/mL IL-2) and subsequently transduced with AAV6 containing each donor template at 5% by volume. After Day 14 post-isolation cells were switched to RPMI+10% FBS (with 300 IU/mL IL-2) and rested for 3 days. Lentiviral transduced cells were transduced at an MOI of 12 on day 3. On Day 17 post-isolation Tregs and Teff were CFSE labeled and plated with either CTV labeled allogeneic (FIGS. 13A-D) or CTV labeled autologous (FIGS. 13E-F) responder T cells (total T cells) isolated from frozen PBMC that day. 50,000 responders per well were utilized along with varying ratios of Treg/Teff and 1:10 Tresp:CTS anti CD3/28 dynabeads for 3 days, after which proliferation (FIGS. 13A, B, E, and F) was determined by flow cytometry and supernatants were collected for 6plex Luminex (read out on Magpix; FIGS. 13 C, D, G, and H).

### I. Example 9: FOXP3 Gene Edited Samples Show Increased Resistance to Pro-Inflammatory Culture Induced Destabilizing Conditions

This example demonstrates that cells edited to comprise a CAR inserted in the FOXP3 locus under the control of a FOXP3 promoter show increased resistance to pro-inflammatory culture induced destabilizing conditions.

Primary Treg cells were edited at Day 3 post-isolation by nucleofection with Cas9: Guide RNP, recovered in 500 μL OpTmizer media (with 3001U/mL IL-2) and subsequently transduced with AAV6 containing each donor template at 5% by volume. Cells were enriched by FACS to high purity on Day 14 post-isolation after which cells were switched to RPMI+10% FBS (with 300 IU/mL IL-2) and rested for 3 days. To model inflammatory Treg destabilizing conditions cells were then cultured in RPMI assay media for 21 additional days in the presence of 300 IU/mL IL-2 only, or 300 IU/mL IL-2, 50 ng/mL IL-1 (E≤, IL-5, and IL-23 and stimulated with CTS anti-CD3/28 dynabeads weekly. Cells were de-beaded on D14 post-expansion, and restimulated the same day. Various markers were assessed by flow cytometry on days 0, 3, 7, 1, 14, 17, and 21, including FOXP3 and HELIOS.

FIG. 14 shows that HDR start and non-edited Treg cells demonstrate similar FOXP3+HELIOS+ Treg phenotypes (y-axis) in both IL-2 only and inflammatory conditions over the course of 21 days.

This example demonstrates that cells comprising a transgene under the control of a FOXP3 promoter show increased resistance to pro-inflammatory culture induced destabilizing conditions.

### J. Example 10: FOXP3 Gene Edited Samples Show Increased Resistance to Pro-Inflammatory Culture Induced Destabilizing Conditions

This example demonstrates that cells edited to comprise a CAR inserted in the FOXP3 locus under the control of a FOXP3 promoter show increased resistance to pro-inflammatory culture induced destabilizing conditions.

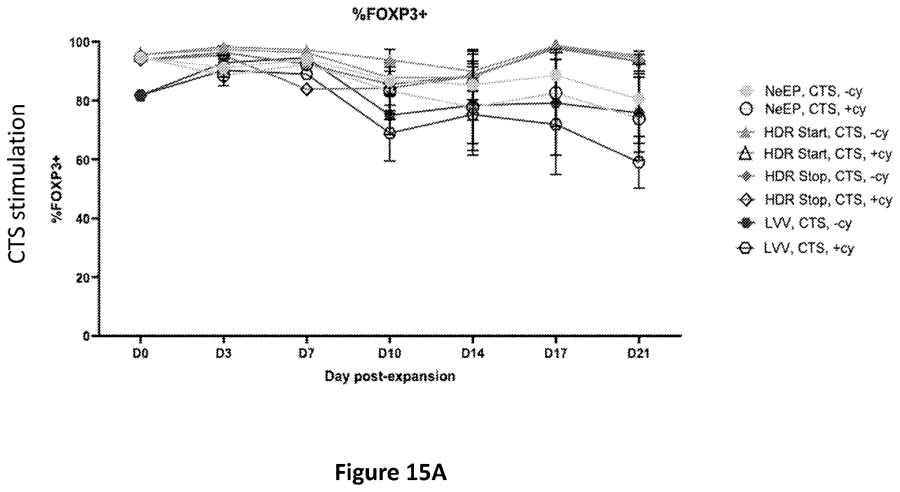

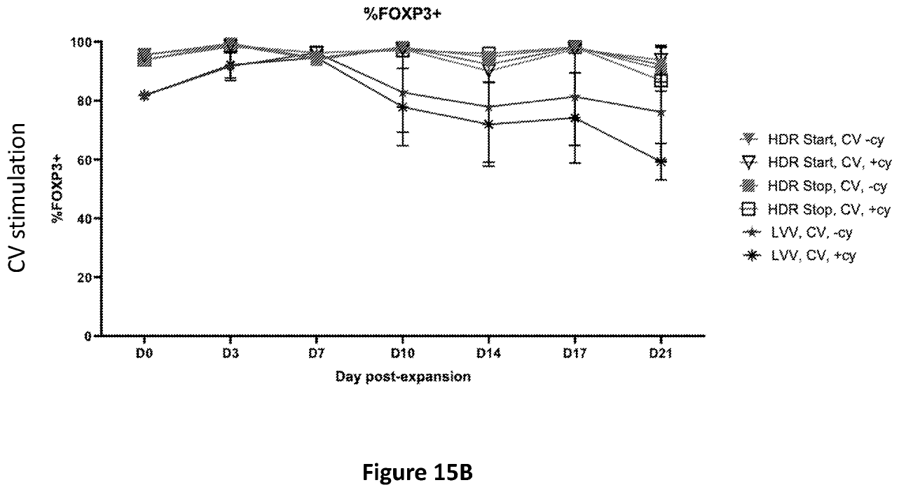

Primary Treg cells were edited at Day 3 post-isolation by nucleofection with Cas9: Guide RNP, recovered in 500 μL OpTmizer media (with 300 IU/mL IL-2) and subsequently transduced with AAV6 containing each donor template at 5% by volume. Cells were enriched by FACS to high purity on Day 14 post-isolation after which cells were switched to RPMI+10% FBS (with 3001U/mL IL-2) and rested for 3 days. To model inflammatory Treg destabilizing conditions cells were then cultured in RPMI assay media for 21 additional days in the presence of 300 IU/mL IL-2 only, or 300 IU/mL IL-2, 50 ng/mL IL-1β, IL-5, and IL-23 and stimulated with CTS anti-CD3/28 dynabeads (FIG. 15A), or antigen specific (CV; FIG. 15B) beads weekly. Cells were de-beaded on D14 post-expansion, and restimulated the same day. Various markers were assessed by flow cytometry on days 0, 3, 7, 1, 14, 17, and 21, including FOXP3 and HELIOS.

FIG. 15A shows that HDR start, HDR stop, and non-edited Treg cells demonstrate similar FOXP3+ Treg phenotypes (y-axis) in the presence and absence of inflammatory when stimulated with CTS anti-CD3/28 dynabeads conditions over the course of 21 days.

FIG. 15B shows that HDR start, HDR stop, and non-edited Treg cells demonstrate similar FOXP3+ Treg phenotypes (y-axis) in the presence and absence of inflammatory when stimulated with antigen specific (CV) dynabeads conditions over the course of 21 days.

This example demonstrates that cells comprising a transgene under the control of a FOXP3 promoter show increased resistance to pro-inflammatory culture induced destabilizing conditions.

### K. Example 11: FOXP3 Gene Edited Samples Show Increased Resistance to Pro-Inflammatory Culture Induced Destabilizing Conditions

This example demonstrates that cells edited to comprise a CAR in the FOXP3 locus under the control of a FOXP3 promoter show increased resistance to pro-inflammatory culture induced destabilizing conditions.

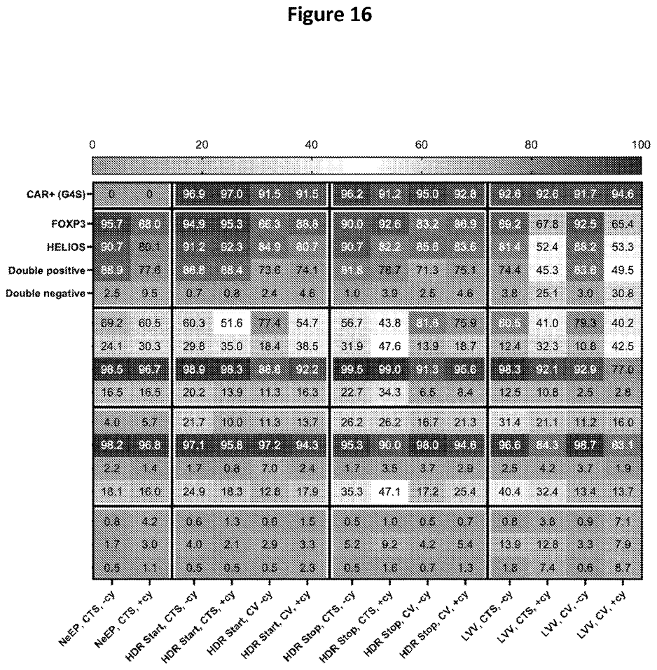

Primary Treg cells were edited at Day 3 post-isolation by nucleofection with Cas9: Guide RNP, recovered in 500 μL OpTmizer media (with 300 IU/mL IL-2) and subsequently transduced with AAV6 containing each donor template at 5% by volume. Cells were enriched by FACS to high purity on Day 14 post-isolation after which cells were switched to RPMI+10% FBS (with 300 IU/mL IL-2) and rested for 3 days. To model inflammatory Treg destabilizing conditions cells were then cultured in RPMI assay media for 21 additional days in the presence of 300 IU/mL IL-2 only, or 300 IU/mL IL-2, 50 ng/ml IL-1β, IL-5, and IL-23 and stimulated with CTS anti-CD3/28 dynabeads weekly. Cells were de-beaded on D14 post-expansion and restimulated the same day. Various markers were assessed by flow cytometry on days 0, 3, 7, 1, 14, 17, and 21, including FOXP3 and HELIOS. Heatmap (FIG. 16) shows markers at the end of post-expansion protocol. Samples are divided by engineering condition (Non-edited electroporated, FOXP3 start gene editing, FOXP3_Stop gene editing, and Lentiviral vector), stimulation type (anti-CD3/28 CTS bead, CV anti-idiotype bead), and cytokine media condition (IL-2 only without proinflammatory cytokines, or IL-2 with proinflammatory cytokines). CAR positivity is gated off of live, and the remainder of markers are gated off the CAR+ fraction.

### VII. EQUIVALENTS

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The terms “a” and “an” and “the” and similar references in the context of describing the invention (especially in the context of the following claims) are to be construed to cover both the singular and the plural, unless otherwise indicated herein or clearly contradicted by context. For example, the term “a cell” includes a plurality of cells, including mixtures thereof. Where the plural form is used for compounds, salts, or the like, this is taken to mean also a single compound, salt, or the like.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A cell comprising a polynucleotide in a FOXP3 locus comprising a nucleotide sequence comprising a promoter, a FOXP3 gene or a functional derivative thereof, and a transgene, wherein expression of the FOXP3 gene or functional derivative thereof and the transgene are under the control of the promoter.

2. The cell of claim 1, wherein the nucleotide sequence further comprises a heterologous 3′ untranslated region (UTR).

3. The cell of claim 1 or claim 2, wherein the promoter is a FOXP3 promoter.

4. The cell of any one of claims 1-3, wherein the FOXP3 locus comprises a safe harbor site.

5. The cell of any one of claims 1-4, wherein the transgene is inserted upstream of a start codon of the FOXP3 gene.

6. The cell of claim 5, wherein the transgene is inserted about 61 to 1 bases before the start codon of the FOXP3 gene.

7. The cell of claim 6, wherein the transgene is inserted immediately upstream of the start codon of the FOXP3 gene.

8. The cell of any one of claims 1-4, wherein the transgene is inserted downstream of the FOXP3 gene.

9. The cell of claim 8, wherein the transgene is inserted about 61 to 1 bases after the stop codon of the FOXP3 gene.

10. The cell of claim 9, wherein the transgene is inserted immediately after the stop codon of the FOXP3 gene.

11. The cell of any one of claims 8-10, wherein the stop codon of the FOXP3 gene is engineered to encode an amino acid or is deleted.

12. The cell of any one of claims 1-11, wherein the FOXP3 gene encodes a full length foxp3 polypeptide.

13. The cell of any one of claims 1-12, wherein the nucleotide sequence further encodes a self-cleaving peptide between the FOXP3 gene and the transgene.

14. The cell of claim 13, wherein the self-cleaving peptide (SCP) comprises a 2A peptide.

15. The cell of claim 14, wherein the 2A peptide comprises a P2A, E2A, F2A, or T2A peptide.

16. The cell of any one of claims 1-15, wherein the FOXP3 gene and the transgene are expressed as a single polypeptide.

17. The cell of claim 16, wherein the polypeptide comprises from N- to C-terminus: transgene-SCP-foxp3.

18. The cell of claim 16, wherein the polypeptide comprises from N- to C-terminus: foxp3-SCP-transgene.

19. The cell of claim 17 or claim 18, further comprising a foxp3 polypeptide comprising a first portion of the self-cleaving peptide and a polypeptide encoded by the transgene comprising a second portion of the self-cleaving peptide.

20. The cell of any one of claims 1-12, wherein the FOXP3 gene and the transgene are expressed as separate polypeptides.

21. The cell of claim 20, wherein the nucleotide sequence further comprises an internal ribosome entry site (IRES) between the FOXP3 gene and the transgene.

22. The cell of any one of claims 1-21, wherein the transgene (TG) encodes a cytokine, interleukin, cytokine receptor, transcription factor, and/or chimeric receptor.

23. The cell of claim 22, wherein the transgene encodes a chimeric antigen receptor (CAR) or CAR component and/or a T-cell receptor (TCR).

24. The cell of claim 22 or 23, wherein the transgene encodes a chemokine receptor.

25. The cell of claim 24, wherein the chemokine receptor (CR) comprises a CCR or CXCR receptor.

26. The cell of claim 25, wherein the CCR receptor comprises CCR7 and/or CCR9.

27. The cell of any one of claims 24-26, wherein the polypeptide expressed from the FOXP3 locus comprises from N- to C-terminus: TG-SCP-foxp3.

28. The cell of claim 27, wherein the polypeptide expressed from the FOXP3 locus comprises from N- to C-terminus: CAR-SCP₁-TG-SCP₂-foxp3.

29. The cell of claim 28, wherein the wherein the polypeptide expressed from the FOXP3 locus comprises from N- to C-terminus: CAR-SCP₁-CR-SCP₂-foxp3.

30. The cell of claim 28 or 29, wherein the SCP₁ and SCP₂ are different.

31. The cell of any one of claims 2-30, wherein the heterologous 3′ UTR comprises one or more of the following 3′ UTRs
(i) SV40 reversed,
(ii) bGH,
(iii) WPRE,
(iv) WPRE3/SV40p128,
(v) SV40 corrected,
(vi) WPRE3/SV40.2,
(vii) TCRA EIF3, or
(viii) s27a3RU.

32. The cell of claim 31, wherein the 3′ UTR comprises a SV40 reversed, SV40 corrected, or bGH 3′UTR.

33. The cell of any one of claims 1-32, wherein the FOXP3 gene further comprises one or more mutations in a codon encoding a ubiquitination or deubiquitination site.

34. The cell of claim 33, wherein the ubiquitination or deubiquitination site comprises codon 227, 250, 263, 268, and/or 422.

35. The cell of any one of claims 1-34, wherein the cell comprises a eukaryotic cell.

36. The cell of claim 35, wherein the eukaryotic cell comprises a metazoan cell.

37. The cell of claim 35 or claim 36, wherein the eukaryotic cell comprises a human cell.

38. The cell of any one of claims 35-37, wherein the cell comprises an immune cell or an immune precursor cell.

39. The cell of claim 38, wherein the immune cell comprises a neutrophil, eosinophil, basophil, mast cell, monocyte, macrophage, dendritic cell, natural killer cell, T cell, or a lymphocyte.

40. The cell of claim 39, wherein the immune cell comprises a T cell.

41. The cell of claim 40, wherein the T cell comprises an immunosuppressive T cell.

42. The cell of claim 41, wherein the immunosuppressive T cell comprises a regulatory T cell (Treg).

43. The cell of claim 38, wherein the immune precursor cell comprises a stem cell, an induced pluripotent stem cell, a hematopoietic stem cell, or a CD4⁺ stem cell.

44. The cell of any one of claims 36-43, wherein the cell is isolated from an individual.

45. The cell of claim 44, wherein the individual is suffering from a disease.

46. The cell of claim 45, wherein the disease comprises an autoimmune disease.

47. The cell of claim 46, wherein the autoimmune disease comprises intestinal inflammation.

48. The cell of claim 47, wherein the autoimmune disease comprises inflammatory bowel disease (IBD).

49. The cell of claim 48, wherein the autoimmune disease comprises autoimmune disease comprises Crohn's disease (CD) or ulcerative colitis (UC).

50. The cell of claim 49, wherein the autoimmune disease comprises CD.

51. The cell of claim 49, wherein the autoimmune disease comprises UC.

52. The cell of claim 44, wherein the cell is isolated from a healthy individual.

53. The cell of claim 52, wherein the cell is engineered to not promote an immune response in an allogeneic individual.

54. The cell of any one of claims 22-53, further comprising a CAR polypeptide expressed on a surface of the cell.

55. The cell of any one of claims 25-54, further comprises a CR polypeptide expressed on a surface of the cell.

56. A cell comprising a polynucleotide in a FOXP3 locus comprising a nucleotide sequence comprising a promoter and a transgene encoding a chimeric antigen receptor (CAR) and a chemokine receptor (CR), wherein expression of the transgene is under the control of the promoter.

57. The cell of claim 56, wherein the nucleotide sequence further comprises a heterologous 3′ untranslated region (UTR).

58. The cell of claim 56 or claim 57, wherein the promoter is a FOXP3 promoter.

59. The cell of any one of claims 56-58, wherein the nucleotide sequence further comprises a FOXP3 gene or a functional derivative thereof, wherein expression of the FOXP3 gene or functional derivative thereof is under the control of the FOXP3 promoter.

60. The cell of claim 59, wherein the FOXP3 gene encodes a full length foxp3 polypeptide.

61. A cell comprising a polynucleotide in a FOXP3 locus comprising a nucleotide sequence comprising a promoter, a FOXP3 gene or a functional derivative thereof, and a heterologous 3′ untranslated region (UTR), wherein expression of the FOXP3 gene or functional derivative thereof is under the control of the promoter.

62. The cell of claim 61, wherein the promoter comprises a FOXP3 promoter.

63. The cell of claim 61 or claim 62, wherein the FOXP3 gene encodes a full length foxp3 polypeptide.

64. The cell of any one of claims 61-63, wherein the nucleotide sequence further comprises a transgene, wherein expression of the transgene is under the control of the FOXP3 promoter.

65. The cell of claim 64, wherein the transgene encodes a chimeric antigen receptor (CAR) or CAR component.

66. The cell of claim 64 or claim 65, wherein the transgene encodes a self-cleaving peptide.

67. The cell of any one of claims 64-66, wherein the transgene encodes a cytokine, interleukin, cytokine receptor, transcription factor, and/or chimeric receptor.

68. The cell of claim 67, wherein the transgene encodes a chemokine receptor.

69. A cell comprising a polynucleotide in a FOXP3 locus comprising a nucleotide sequence comprising a FOXP3 gene or a functional derivative thereof and a heterologous 5′ and/or 3′ UTR, wherein expression of the FOXP3 or functional derivative thereof is under the control of the promoter.

70. The cell of claim 69, wherein the FOXP3 gene encodes a full length foxp3 polypeptide.

71. The cell of claim 69 or claim 70, wherein the nucleotide sequence further comprises a transgene, wherein expression of the transgene is under the control of the promoter.

72. The cell of claim 71, wherein the promoter is a FOXP3 promoter.

73. The cell of claim 71 or claim 72, wherein the transgene (TG) encodes a cytokine, interleukin, cytokine receptor, transcription factor, and/or chimeric receptor.

74. The cell of claim 73, wherein the transgene encodes a chimeric antigen receptor (CAR) or CAR component.

75. The cell of claim 73 or claim 74, wherein the transgene encodes a chemokine receptor.

76. The cell of any one of claims 71-73, wherein the transgene encodes a self-cleaving peptide.

77. A derivative of or a progeny of a cell of any one of claims 1-76.

78. Use of a cell of any one of claims 1-76, to treat an individual suffering from a disease.

79. The cell of claim 77, wherein the disease comprises an autoimmune disease.

80. The cell of claim 79, wherein the autoimmune disease comprises intestinal inflammation.

81. The cell of claim 80, wherein the autoimmune disease comprises inflammatory bowel disease (IBD).

82. The cell of claim 81, wherein the autoimmune disease comprises autoimmune disease comprises Crohn's disease (CD) or ulcerative colitis (UC).

83. The cell of claim 82, wherein the autoimmune disease comprises CD.

84. The cell of claim 82, wherein the autoimmune disease comprises UC.

85. A composition comprising:
(1) a nucleic acid-guided nuclease system comprising:
(a) a nucleic acid-guided nuclease, or a polynucleotide encoding the nuclease; and
(b) a guide nucleic acid (gNA), or a polynucleotide encoding the gNA,
comprising a spacer sequence complementary to a site within or near the FOXP3 gene; and
(2) a donor template encoding a transgene.

86. The composition of claim 85, wherein the site is upstream of a start codon of the FOXP3 gene.

87. The composition of claim 86, wherein the site is about 61 to 1 bases before the start codon of the FOXP3 gene.

88. The composition of claim 87, wherein the site is immediately upstream of the start codon of the FOXP3 gene.

89. The composition of claim 85, wherein the site is downstream of the FOXP3 gene.

90. The composition of claim 89, wherein the site is about 61 to 1 bases after the stop codon of the FOXP3 gene.

91. The composition of claim 90, wherein the site is immediately after the stop codon of the FOXP3 gene.

92. The composition of any one of claims 85-91, wherein the transgene encodes a cytokine, interleukin, cytokine receptor, transcription factor, and/or chimeric receptor.

93. The composition of claim 92, wherein the transgene encodes a CAR or CAR component and/or a T-cell receptor.

94. The composition of claim 93, wherein the transgene encodes a CAR or a CAR component.

95. The composition of any one of claims 92-94, wherein the transgene encodes a cytokine receptor.

96. The composition of any one of claims 92-95, wherein the transgene encodes a self-cleaving peptide.

97. The composition of claim 96, wherein the self-cleaving peptide comprises a 2A peptide.

98. The composition of claim 97, wherein the 2A peptide comprises a P2A, E2A, F2A, or T2A peptide.

99. The composition of any one of claims 85-98, wherein the spacer sequence comprises any one of SEQ ID NOs: 1-14.

100. The composition of any one of claims 85-99, wherein the nucleic acid-guide nuclease comprises a Class 1 or Class 2 nuclease.

101. The composition of claim 100, wherein the nucleic acid-guided nuclease comprises a Type II or Type V nuclease.

102. The composition of claim 101, wherein the nucleic acid-guided nuclease comprises cas9.

103. The composition of claim 101, wherein the nucleic acid-guided nuclease comprises a Type V-A, V-B, V-C, V-D, or V-E nuclease.

104. The composition of any one of claims 85-103, wherein the nuclease comprises one or more nuclear localization signals.

105. The composition of any one of claims 85-104, wherein the gNA comprises a gRNA.

106. The composition of any one of claims 85-105, wherein nucleic acid-guided nuclease complex and the gNA are present as a nucleic acid-guided nuclease complex.

107. A cell comprising a composition of any one of claims 85-106, or a derivative or progeny thereof.

108. The cell of 107, wherein the cell comprises a eukaryotic cell.

109. The cell of claim 108, wherein the eukaryotic cell comprises a metazoan cell.

110. The cell of claim 108 or claim 109, wherein the eukaryotic cell comprises a human cell.

111. The cell of any one of claims 107-110, wherein the cell comprises an immune cell or an immune precursor cell.

112. The cell of claim 111, wherein the immune cell comprises a neutrophil, eosinophil, basophil, mast cell, monocyte, macrophage, dendritic cell, natural killer cell, T cell, or a lymphocyte.

113. The cell of claim 112, wherein the immune cell comprises a T cell.

114. The cell of claim 113, wherein the T cell comprises an immunosuppressive T cell.

115. The cell of claim 114, wherein the immunosuppressive T cell comprises a regulatory T cell (Treg).

116. The cell of claim 111, wherein the immune precursor cell comprises a stem cell, an induced pluripotent stem cell, a hematopoietic stem cell, or a CD4⁺ stem cell.

117. The cell of any one of claims 109-116, wherein the cell is isolated from an individual.

118. The cell of claim 117, wherein the individual is suffering from a disease.

119. The cell of claim 118, wherein the disease comprises an autoimmune disease.

120. The cell of claim 119, wherein the autoimmune disease comprises intestinal inflammation.

121. The cell of claim 120, wherein the autoimmune disease comprises inflammatory bowel disease (IBD).

122. The cell of claim 121, wherein the autoimmune disease comprises autoimmune disease comprises Crohn's disease (CD) or ulcerative colitis (UC).

123. The cell of claim 122, wherein the autoimmune disease comprises CD.

124. The cell of claim 122, wherein the autoimmune disease comprises UC.

125. The cell of claim 117, wherein the cell is isolated from a healthy individual.

126. The cell of claim 125, wherein the cell is engineered to not promote an immune response in an allogeneic individual.

127. Use of a composition of any one of claims 85-106 to generate a cell of any one of claims 1-84.

128. A method for editing a genome of a cell comprising contacting the cell with a composition of any one of claims 85-106, whereby the resulting cell comprises a heterologous polynucleotide in a FOXP3 locus.

129. The method of 128, wherein the cell comprises a eukaryotic cell.

130. The method of claim 129, wherein the eukaryotic cell comprises a metazoan cell.

131. The method of claim 129 or claim 130, wherein the eukaryotic cell comprises a human cell.

132. The method of any one of claims 128-131, wherein the cell comprises an immune cell or an immune precursor cell.

133. The method of claim 132, wherein the immune cell comprises a neutrophil, eosinophil, basophil, mast cell, monocyte, macrophage, dendritic cell, natural killer cell, T cell, or a lymphocyte.

134. The method of claim 133, wherein the immune cell comprises a T cell.

135. The method of claim 134, wherein the T cell comprises an immunosuppressive T cell.

136. The method of claim 135, wherein the immunosuppressive T cell comprises a regulatory T cell (Treg).

137. The method of claim 132, wherein the immune precursor cell comprises a stem cell, an induced pluripotent stem cell, a hematopoietic stem cell, or a CD4+ stem cell.

138. The method of any one of claims 128-137, wherein, after editing, expression of foxp3 and/or the transgene in the cell is at least 1.1, 1.2, 1.3, 1.4, 1.5, 1.6, 1.7, 1.8, 1.9, 2, 2.2, 2.4, 2.6, 2.8, 3, 3.5, 4, or 4.5-fold and/or not more than 1.2, 1.3, 1.4, 1.5, 1.6, 1.7, 1.8, 1.9, 2, 2.2, 2.4, 2.6, 2.8, 3, 3.5, 4, 4.5, or 5-fold greater than a cell comprising the wildtype FOXP3 3′ UTR, for example 1.1 to 5-fold, preferably 1.1 to 3-fold, more preferably 1.5 to 2-fold.

139. The method of any one of claims 128-138, wherein, after editing, the cell demonstrates no more than 50, 40, 30, 20, 10, 9, 8, 7, 6, 5, 4, 3, 2, 1 or, 0% expression of a transgene within FOXP3 when measured as an effector T-cell as compared to when measured as an immunosuppressive T-cell.

140. A composition comprising a cell generated with a method of any one of claims 128-139, a derivative thereof, or a progeny thereof.

141. A method of editing genomes of a plurality of cells comprising contacting the plurality with a composition of any one of claims 85-106, whereby the resulting cell comprises a heterologous polynucleotide in FOXP3.

142. The method of claim 141, wherein the plurality of cells are isolated from an individual.

143. The method of any one of claim 141 or claim 142, wherein isolating further comprise purifying the cells such that the plurality of cells comprises at least 50, 60, 70, 80, 85, 90, 95, 96, 97, 98, 99, 99.5, or 99.9% immunosuppressive T-cells.

144. The method of any one of claims 141-143, wherein, after editing, expression of foxp3 and/or the transgene in the cell is at least 1.1, 1.2, 1.3, 1.4, 1.5, 1.6, 1.7, 1.8, 1.9, 2, 2.2, 2.4, 2.6, 2.8, 3, 3.5, 4, or 4.5-fold and/or not more than 1.2, 1.3, 1.4, 1.5, 1.6, 1.7, 1.8, 1.9, 2, 2.2, 2.4, 2.6, 2.8, 3, 3.5, 4, 4.5, or 5-fold greater than a cell comprising the wildtype FOXP3 3′ UTR, for example 1.1 to 5-fold, preferably 1.1 to 3-fold, more preferably 1.5 to 2-fold.

145. The method of any one of claims 141-144, wherein, after editing, the cell demonstrates no more than 50, 40, 30, 20, 10, 9, 8, 7, 6, 5, 4, 3, 2, 1 or, 0% expression of a transgene within the FOXP3 locus when measured as an effector T-cell as compared to when measured as an immunosuppressive T-cell.

146. A composition comprising a plurality of cells generated with a method of any one of claims 141-145.

147. The composition of claim 146, wherein the plurality of cells comprises at least 80, 85, 90, 95, 96, 97, 98, 99, or 100% of all of the cells in the composition.

148. A composition comprising:
(1) a nucleic acid-guided nuclease system comprising:
(a) a nucleic acid-guided nuclease, or a polynucleotide encoding the nuclease; and
(b) a guide nucleic acid (gNA) comprising a nucleotide sequence at least 80, 85, 90, 95, 96, 97, 98, 99, or 100% identical to any one of SEQ ID NOs: 1-14; and
(2) a donor template encoding a transgene.
